(19) United States
(12) Patent Application Publication
(10) Pub. No.: US 2023/0287046 A1
(43) Pub. Date: Sep. 14, 2023

(54) MOLECULES TARGETING PROTEINS

(71) Applicants: AELIN THERAPEUTICS, GASTON GREEENSLAAN 1 (BE); VIB VZW, Gent (BE); KATHOLIEKE UNIVERSITEIT LEUVEN, Leuven (BE)

(72) Inventors: Filip Maria Hendrik CLAES, Bierbeek (BE); Joost SCHYMKOWITZ, Meensel-Kiezegem (BE); Frederic ROUSSEAU, Sint-Martens (BE); Els Anna Alice BEIRNAERT, Bellem (BE)

(21) Appl. No.: 17/800,844

(22) PCT Filed: Feb. 19, 2021
(86) PCT No.: PCT/EP2021/054121

§ 371 Date: Aug. 18, 2022

(30) Foreign Application Priority Data
Feb. 19, 2020 (EP) EP 20158310.1

(51) Int. Cl. C07K 7/08; A61P 35/00; C12N 15/85

(52) U.S. Cl. CPC C07K 7/08; A61P 35/00; C12N 15/85; C12N 2800/107

(57) ABSTRACT

Aspects of the invention concern non-naturally occurring molecules capable of downregulating the amount or biological activity specifically of mutant or variant forms of a protein, as well applications thereof.

### FIELD

The invention broadly concerns molecules and compositions suitable for downregulating proteins in vitro or in vivo, which can be applied in a variety of areas, including in the medical or veterinary fields, or in the agricultural or horticultural fields. The application also teaches methods for making and using the molecules and compositions comprising the molecules.

### BACKGROUND

Proteins in nature frequently display sequence variation, which produces variant or mutant forms of such proteins having distinct amino acid sequences. In one example, sequence variation may result from an alternative splicing of a protein's pre-mRNA, such that the eventual mRNA molecules are composed of different subsets of protein-coding exons. In another example, sequence variation at a given amino acid position or positions of a protein may be due to sequence variation in the nucleic acid sequence of the corresponding gene which affects the codon or codons encoding said amino acid or amino acids. Nucleic acid sequence variation at a given locus may be due to the polymorphic nature of that locus, i.e., the occurrence of two or more genetically determined alternative sequences or alleles at that locus in a natural population; or may be the consequence of a hereditary or de novo mutation at that locus, wherein such mutation may in certain instances cause or be associated with a phenotype alteration, such as a detrimental phenotype alteration, more particularly a disease or a disorder. One example are mutations in proto-oncogenes, which can deregulate the proliferation of cells and cause neoplastic diseases, such as cancer. Nucleic acid sequence variations or mutations may encompass both germline and somatic ones.

WO 2007/071789A1 and WO2012/123419A1 describe technology allowing for targeted downregulation of proteins of interest, utilising de novo designed peptide-based molecules (referred to therein as ‘interferors’) comprising at least one β-aggregating sequence which is directed to and can interact with a corresponding β-aggregation prone region (APR) in a protein of interest. Such APRs can be determined in protein sequences using publically available algorithms and computer programs, such as TANGO (Fernandez-Escamilla et al. Nat Biotechnol. 2004, vol. 22, 1302-6, http://tango.embl.de/) or Zyggregator (Pawar et al. J Mol Biol. 2005, vol. 350, 379-92; Tartaglia and Vendruscolo, Chem Soc Rev. 2008, vol. 37, 1395-401).

It was proposed in WO 2007/071789A1 and WO2012/123419A1 that upon contact between a protein of interest comprising an APR in its amino acid sequence and an interferor molecule comprising a β-aggregating sequence corresponding to said APR, a specific n-sheet interaction and co-aggregation occurs between the interferor and the protein of interest, leading to reduced solubility of the protein of interest and its sequestration into aggregates or inclusion bodies, and consequently an effective down-regulation or knock-down of the biological function of said protein of interest.

### SUMMARY

The present invention is at least in part based on the inventors' insight that certain amino acid sequence variations or mutations in a protein can modify the profile of β-aggregation prone regions (APRs) in said protein such that it becomes possible to design novel molecules which specifically target the variant or mutant forms of the protein for downregulation. For example, a sequence variation or mutation in a protein may modify the amino acid sequence and/or the aggregation propensity of a pre-existing APR in that protein, and this difference in APR properties can be exploited to design novel molecules targeting specifically the APR in the variant or mutant form of the protein. In another example, a sequence variation or mutation in a protein may introduce a new (de novo) APR where, absent said variation or mutation, the protein did not contain a corresponding APR. This may occur for instance when an additional amino acid sequence containing the APR is inserted into the protein, such as by alternative splicing or by an insertion mutation; or when an amino acid stretch that to some extent approximates but does not yet qualify as an APR is modified by the variation or mutation so that it can be qualified as an APR.

Accordingly, an aspect provides a non-naturally occurring molecule capable of downregulating the amount or biological activity of a mutant or variant form of a protein, wherein:
a) the protein comprises a β-aggregation prone region (APR) and said APR is modified by the mutation or variation in the mutant or variant form of the protein; or
b) the mutation or variation introduces a de novo APR in the mutant or variant form of the protein not present in the protein;
and wherein the molecule is configured to specifically target the APR in the mutant or variant form of the protein.

Further aspects provide any molecule as taught herein for use in medicine, including in human or veterinary medicine, i.e., in treating humans or animals. Further aspects provide any molecule as taught herein for use in a method of treating a disease caused by or associated with the mutant or variant form of the protein. Related aspects provide a method for treating a subject in need thereof, the method comprising administering to the subject a therapeutically effective amount of any molecule as taught herein. Further related aspects provide a method for treating a subject having a disease caused by or associated with the mutant or variant form of the protein, the method comprising administering to the subject a therapeutically effective amount of any molecule as taught herein.

Further aspects provide a pharmaceutical composition comprising any molecule as taught herein.

Further aspects provide an in vitro method for downregulating the amount or biological activity of a mutant or variant form of a protein in a cell expressing, preferably endogenously expressing, the mutant or variant form of the protein, the method comprising contacting the cell with any molecule as taught herein.

Further aspects provide a method for downregulating the amount or biological activity of a mutant or variant form of a protein in an organism expressing, preferably endogenously expressing, the mutant or variant form of the protein, the method comprising administering to the organism any molecule as taught herein.

It shall be appreciated that the present molecules are broadly applicable in many technical fields or areas, in which preferential detection or targeting of mutant or variant protein forms may be of interest, for example to detect or reduce the expression and biological activity of the mutant or variant protein in an organism of interest, or in a pathogen of such organism. Such fields include, without limitation, medical and veterinary practice, diagnostics, research tools, agriculture, horticulture, aquaculture, and others.

These and further aspects and preferred embodiments of the invention are described in the following sections and in the appended claims. The subject-matter of the appended claims is hereby specifically incorporated in this specification.

### BRIEF DESCRIPTION OF DRAWINGS

FIG. 1 illustrates a screen of RAS-targeting molecules (‘pept-ins’) according to certain embodiments of the present invention on NCI-H441 tumor cell line cultures. (A) Single-dose (25 μM) screen of RAS-targeting pept-ins on adherently growing (2D) NCI-H441 cells. Viability was assessed after 4 days of exposure to the test compounds and normalized to the vehicle condition (30 mM Urea). (B) Single-dose (25 μM) screen of RAS-targeting pept-ins on NCI-H441 cells growing as suspension spheroid cultures (3D). Viability was assessed after 5 days of exposure to the test compounds and normalized to the vehicle condition (30 mM Urea). NT: Not tested. Error bars represent the SD.

FIG. 2 illustrates dose-response and IC₅₀ determination of RAS-targeting molecules (‘pept-ins’) according to certain embodiments of the present invention and a negative control. Pept-ins were tested in a five-point dose-response using a one-in-two dilution series starting from 50 μM as highest dose on adherently growing (2D) NCI-H441 cells. Viability was assessed after three days of exposure to the test compounds and normalized to vehicle conditions. Error bars represent the SD.

FIG. 3 illustrates IC₅₀s of RAS-targeting molecules (‘pept-ins’) according to certain embodiments of the present invention on suspension spheroid cultures. Waterfall plots showing the median IC₅₀s of RAS-targeting pept-ins on suspension spheroid cultures. Pept-ins were tested in a five-point dose-response using a one-in-two dilution series starting from 50 μM as highest dose on spheroid suspension cultures on a set of cell lines with different KRAS mutations. Viability was assessed five days after of exposure to the test compounds. Error bars represent the SD on the median, if applicable.

FIG. 4 illustrates kinetic tinctorial aggregation assays on RAS-targeting molecules (‘pept-ins’) according to certain embodiments of the present invention. Aggregation behaviour of the RAS-targeting pept-ins was studied by performing kinetic tinctorial assays using the amyloid aggregate sensor dyes Thioflavin T (ThT; lower panel) and pentameric formyl thiophene acetic acid (p-FTAA; upper panel). All four biologically active pept-ins showed clear amyloid-aggregation kinetics with both dyes, while the inactive control showed no significant ThT signal and only a slight increase in p-FTAA signal over time.

FIG. 5 illustrates seeding of KRAS G12V by RAS-targeting molecules (‘pept-ins’) according to certain embodiments of the present invention. Seeding experiments of recombinant native KRAS G12V protein was performed with end-stage aggregates (left panels) or sonicated seeds (right panels) of the different KRAS-targeting pept-ins. To this end, pept-ins were allowed to aggregate for 22 hrs. End-stage samples were mixed with recombinant KRAS G12V and aggregation was monitored kinetically using ThT. This approach revealed only minor seeding capacity of these end-stage pept-in aggregates on KRAS G12V. However, upon disruption of the mature aggregates through sonication, potent seeds are formed which efficiently induce aggregation of KRAS G12V.

FIG. 6 illustrates in vitro translation assay showing target selectivity of RAS-targeting molecules (‘pept-ins’) according to certain embodiments of the present invention. In vitro translation assay producing either wild-type or different mutant KRAS in the presence of biotinylated RAS-targeting pept-ins. Streptavidin pull-down was used to capture the biotinylated pept-ins from the translation reaction and pulled-down fraction was probed for KRAS using Western blot. The biotinylated version of pept-in 04-004-N001, i.e. 04-004-N011, which harbours an APR window sequence derived from a wild-type APR, is predicted to target all RAS proteins independently from their mutation status. While efficient pull-down with 04-004-N001 was indeed observed for KRAS wild-type, G12V and G12C, binding to the G12D and G13D mutants appeared to be less efficient. Using the biotinylated versions of the biologically active pept-ins harbouring an APR window containing the G12V mutant site (04-006-N007, 04-015-N026 and 04-033-N003), however, pull-down was only observed for the G12V mutant KRAS and, in the case of 04-015-N026, for G12C mutant KRAS.

FIG. 7 illustrates cellular co-immunoprecipitation assays showing target engagement by RAS-targeting molecules (‘pept-ins’) according to certain embodiments of the present invention. Cellular target engagement of biotinylated pept-ins was assessed using co-immunoprecipitation assay. NCI-H441 cells were treated with 25 μM biotinylated pept-ins overnight after which pept-ins were immunoprecipitated from the lysates using streptavidin-coated beads. Precipitated fractions were probed for KRAS using Western blot. While this approach yielded no detectable KRAS protein in the precipitated fractions from vehicle or negative control peptide-treated conditions, KRAS protein was readily detected in the precipitated fractions from NCI-H441 cells treated with biologically active pept-ins.

FIG. 8 illustrates cellular co-localization between mCherry-labeled KRAS and FITC-labeled RAS-targeting molecules (‘pept-ins’) according to certain embodiments of the present invention. HeLa cells overexpressing mCherry-tagged KRAS G12V were treated with the RAS-targeting FITC-labeled version of pept-in 04-015-N001 (04-015-N032) and imaged 75 min after initial exposure to the pept-in. mCherry-labeled KRAS associates with the pept-in as revealed by the occurrence of inclusion-like perinuclear structures that are positive for both FITC as well as mCherry (white arrows).

FIG. 9 illustrates that RAS-targeting molecules (‘pept-ins’) according to certain embodiments of the present invention lower solubility and total levels of the KRAS protein. NCI-H441 cells were treated with a near IC50 dose (12.5 μM) and a near 2×IC50 dose (25 μM) for 24 hrs. Insoluble proteins in lysates were collected by centrifugation and both soluble and insoluble protein fractions were probed for KRAS on Western blot. This analysis showed that all biologically active RAS-targeting peptides dose-dependently increased the percentage of KRAS in the insoluble fraction while the percentage of insoluble KRAS was comparable between vehicle and negative control peptide treated samples (A). Quantification of total KRAS levels in these samples (i.e. sum of KRAS levels in the soluble and insoluble fraction for each treatment) showed that total KRAS levels were also dose-dependently reduced in the samples treated with the biologically active RAS-targeting pept-ins (B).

FIG. 10 illustrates mutant-selective cellular efficacy using the RASless MEF panel. Graph showing mean±SD as well as individual assay IC50s from at least three independent experiments assessing the efficacy of the indicated RAS-target pept-ins on a panel of RASless MEFs, expressing either wild-type (WT), mutant G12V or G12C KRAS, or a V600E mutant BRAF in absence of endogenous K-, H-, and NRAS.

FIG. 11 illustrates cellular co-immunoprecipitation assays showing target engagement by RAS-targeting molecules (‘pept-ins’) according to certain embodiments of the present invention. Cellular target engagement of biotinylated pept-ins was assessed using co-immunoprecipitation assay. KRAS wild-type or mutant G12V expressing RASless MEFs. In the RASless MEF-based assay, blots show that the 04-004-derived biotinylated pept-in precipitated both wild-type and mutant G12V KRAS well. The biotinylated versions of the G12V-selective pept-ins, however, show preferential binding to the G12V mutant KRAS protein.

FIG. 12 illustrates flow cytometry assay probing cell death and protein aggregation upon treatment with RAS-targeting pept-ins. NCI-H441 lung adenocarcinoma cells were treated with the indicated RAS-targeting pept-ins and control conditions for 6, 16 or 24 hrs. After treatment, cells were collected and stained for cell death (Sytox™ Blue) and protein aggregation (Amytracker™ Red), and next analyzed on a flow cytometer. Scatter plots show Sytox Blue intensity on the Y-axis and Amytracker Red intensity on the X-axis. Hpt: hours post treatment. Treatment with all of the RAS-targeting pept-ins, but not with the control conditions, induced protein aggregation as evidenced by the increase in Amytracker Red signal. Furthermore, this increase in aggregation appears to result in cell death, as indicated by the slower but parallel increase in Sytox Blue.

FIG. 13 illustrates that RAS-targeting pept-ins reduce tumor growth in a xenograft model of KRAS G12V mutant cancer. A xenograft model of human KRAS G12V mutant colorectal cancer, SW620, was used to assess whether in vivo administration of the RAS-targeting pept-ins resulted in reduction of tumor growth. Pept-ins were dosed 3 times per week by intratumoral injection at either 20 or 200 μg once the tumors reached 100-150 mm³. Model response was monitored by a positive control group receiving Irinotecan at 100 mg/kg, once per week for 3 weeks. Group sizes were N=6 for the non-treated group, N=5 for the vehicle groups and N=8 for the pept-in and positive control groups. Graphs show box plots of tumor volumes at day 22 after treatment started. The displayed graphs demonstrate a significant reduction in tumor volume for 04-004-N001 (200 μg dosing group) and 04-015-N001 (20 g and 200 g dosing groups) by one-way ANOVA.

FIG. 14 illustrates selective binding of pept-ins 22-006-N001 and 22-018-N001, designed against ITK R29C or R29L mutants, to the respective ITK mutants in an in vitro translation assay.

### DESCRIPTION OF EMBODIMENTS

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms also encompass “consisting of” and “consisting essentially of”, which enjoy well-established meanings in patent terminology. That said, as regards the term “consisting essentially of”, by means of further illustration, where a molecule is recited to consist essentially of structural elements A-B-C, the molecule would necessarily include the listed elements and would be open to also contain unlisted structural elements that do not materially affect the basic and novel properties of the molecule.

Hence, where the elements A-B-C were to form the operative part or principle of the molecule, in particular by facilitating the molecule's interaction with or effect on a given target, the term “consisting essentially of” would ensure the presence of said elements A-B-C in the molecule, and would also allow for the presence of unlisted elements which do not materially affect the molecule's interaction with said target.

The recitation of numerical ranges by endpoints includes all numbers and fractions subsumed within the respective ranges, as well as the recited endpoints. This applies to numerical ranges irrespective of whether they are introduced by the expression “from . . . to . . . ” or the expression “between . . . and . . . ” or another expression.

The terms “about” or “approximately” as used herein when referring to a measurable value such as a parameter, an amount, a temporal duration, and the like, are meant to encompass variations of and from the specified value, such as variations of +/−10% or less, preferably +1-5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed.

Whereas the terms “one or more” or “at least one”, such as one or more members or at least one member of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members. In another example, “one or more” or “at least one” may refer to 1, 2, 3, 4, 5, 6, 7 or more.

The discussion of the background to the invention herein is included to explain the context of the invention. This is not to be taken as an admission that any of the material referred to was published, known, or part of the common general knowledge in any country as of the priority date of any of the claims.

Throughout this disclosure, various publications, patents and published patent specifications are referenced by an identifying citation. All documents cited in the present specification are hereby incorporated by reference in their entirety. In particular, the teachings or sections of such documents herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the invention. When specific terms are defined in connection with a particular aspect of the invention or a particular embodiment of the invention, such connotation or meaning is meant to apply throughout this specification, i.e., also in the context of other aspects or embodiments of the invention, unless otherwise defined.

In the following passages, different aspects or embodiments of the invention are defined in more detail. Each aspect or embodiment so defined may be combined with any other aspect(s) or embodiment(s) unless clearly indicated to the contrary. In particular, any feature indicated as being preferred or advantageous may be combined with any other feature or features indicated as being preferred or advantageous.

Reference throughout this specification to “one embodiment”, “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

As corroborated by the experimental section, which illustrates certain representative embodiments of the present invention—in particular, molecules capable of specifically downregulating a human RAS protein carrying a missense mutation but substantially not acting on wild-type human RAS, wherein the design of the molecules exploits the fact that the mutation alters the N-terminal most β-aggregation prone region (APR) of the RAS protein—the inventors now provide the broad teaching that molecules which specifically downregulate the amount or biological activity of variant or mutant forms of proteins can be designed by specifically targeting altered or de novo arising APRs in such variant or mutant forms of proteins.

The ability to specifically target and downregulate a variant or mutant form of a protein may be of particular importance where such variant or mutant form displays properties, functions or effects distinct from the unmodified protein, especially where these properties, functions or effects render the variant or mutant form of the protein detrimental to the health or survival of a cell or an organism. For example, “gain-of-function” mutations may cause a protein to gain a harmful property, function or effect, such as for instance but without limitation they may: increase the activity of a protein or render a protein constitutively, such as a protein involved in cell signalling, active or deregulated; cause a protein to misfold and possibly induce misfolding of other proteins; obstruct normal degradation of a protein; cause a protein to engage in new or stronger protein-protein interactions; or impair the subcellular targeting and localisation of a protein; etc. Further, “dominant negative” mutations may produce a mutant form of a protein which acts antagonistically to the unmodified protein. Hence, not only do such dominant negative mutations impair the function of the mutant protein, but the mutant protein also hampers or eliminates the function of the wild-type protein, for instance by forming an inactive complex with the latter, or by still engaging with cellular partners or in cellular processes as the wild-type protein would but without inducing the normal consequences of such engagement. In certain specific examples, a gain-of-function mutation in a proto-oncogene or a dominant negative mutation in a tumor suppressor gene can endow the mutant protein with the potential to cause or contribute to oncogenic transformation of a cell.

Downregulating the amount or biological activity of variant or mutant forms of proteins can thus be of great value in such and further circumstances. Doing so may for example help to restore the health of a cell or an organism expressing the mutant protein. Or doing so may for example reduce the viability of or kill a cell, wherein the cell is harmful to the organism owing to the expression of the mutant protein by the cell. Accordingly, an aspect provides a non-naturally occurring molecule capable of downregulating the amount or biological activity of a mutant or variant form of a protein, wherein:
a) the protein comprises a β-aggregation prone region (APR) and said APR is modified by the mutation or variation in the mutant or variant form of the protein; or
b) the mutation or variation introduces a de novo APR in the mutant or variant form of the protein not present in the protein;
and wherein the molecule is configured to specifically target the APR in the mutant or variant form of the protein.

The term “non-naturally occurring” generally refers to a material or an entity that is not formed by nature or does not exist in nature. Such non-naturally occurring material or entity may be made, synthesised, semi-synthesised, modified, intervened on or manipulated by man using methods described herein or known in the art. By means of an example, the term when used in relation to a peptide may in particular denote that a peptide of an identical amino acid sequence is not found in nature, or if a peptide of an identical amino acid sequence is present in nature, that the non-naturally occurring peptide comprises one or more additional structural elements such as chemical bonds, modifications or moieties which are not included in and thus distinguish the non-naturally occurring peptide from the naturally occurring counterpart. In certain embodiments, the term when used in relation to a peptide may denote that the amino acid sequence of the non-naturally occurring peptide is not identical to a stretch of contiguous amino acids encompassed by a naturally occurring peptide, polypeptide or protein. For avoidance of doubt, a non-naturally occurring peptide may perfectly contain an amino acid stretch shorter than the whole peptide, wherein the structure of the amino acid stretch including in particular its sequence is identical to a stretch of contiguous amino acids found in a naturally occurring peptide, polypeptide or protein.

In the context of the present disclosure, the phrase “a molecule configured to” intends to encompass any molecule that exhibits the recited outcome or functionality under appropriate circumstances. Hence, the phrase can be seen as synonymous to and interchangeable with phrases such as “a molecule suitable for”, “a molecule having the capacity to”, “a molecule designed to”, “a molecule adapted to”, “a molecule made to”, or “a molecule capable of”.

Any meaningful extent of downregulation of the amount or biological activity of the mutant or variant form of a protein is envisaged. Hence, the terms “downregulate” or “downregulated”, or “reduce” or “reduced”, or “decrease” or “decreased” may in appropriate contexts, such as in experimental or therapeutic contexts, denote a statistically significant decrease relative to a reference. The skilled person is able to select such a reference. An example of a suitable reference may be the amount or activity of the mutant or variant form of the protein when exposed to a ‘negative control’ molecule, such as a molecule of similar composition but known to have no effects on the mutant or variant form of the protein. For example, such decrease may fall outside of error margins for the reference (as expressed, for example, by standard deviation or standard error, or by a predetermined multiple thereof, e.g., 1×SD or ±2×SD, or ±1×SE or ±2×SE). By means of an illustration, the amount or activity of the mutant or variant form of the protein may be considered reduced when it is decreased by at least 10%, such as by at least 20% or by at least 30%, preferably by at least 40%, such as by at least 50% or by at least 60%, more preferably by at least 70%, such as by at least 80% or by at least 90% or more, as compared to the reference, up to and including a 100% decrease.

Any existing, available or conventional separation, detection and/or quantification methods may be used to quantify the amount or biological activity of proteins and thus to determine downregulation thereof, for example in or on a cell, cell population, tissue, organ, or organism. In certain examples, such methods may include biochemical or cell biological assay methods, including inter alia assays of enzymatic activity, membrane channel activity, substance-binding activity, gene regulatory activity, or cell signalling activity of a protein. Such assays may be performed for example on proteins in solution, on proteins in in vitro translation systems, on proteins in cell lysates of cells natively or heterologously expressing the proteins, or on intact or permeabilized cells natively or heterologously expressing the proteins. It shall be understood that the choice of such assays will be determined by the biological activity exhibited by the mutant or variant form of the protein. By means of an example and without limitation, the amount or biological activity of a mutant or variant form of a protein which causes or contributes to the oncogenic behaviour of cells (e.g., an oncogene or a dominant negative form of a tumor suppressor gene), such as a cancer driver protein, can be detected and quantified by measuring the reduction in viability of transformed cell lines which depend for their growth on the oncogenic activity of said mutant or variant form of the protein. In other examples, such methods may include immunological assay methods, wherein the ability to separate, detect and/or quantify a protein is conferred by specific binding between a separable, detectable and/or quantifiable binding agent such as an immunological binding agent (antibody) and the protein. Immunological assay methods include without limitation immunohistochemistry, immunocytochemistry, flow cytometry, mass cytometry, fluorescence activated cell sorting (FACS), fluorescence microscopy, fluorescence based cell sorting using microfluidic systems, immunoaffinity adsorption based techniques such as affinity chromatography, magnetic particle separation, magnetic activated cell sorting or bead based cell sorting using microfluidic systems, enzyme-linked immunosorbent assay (ELISA) and ELISPOT based techniques, radioimmunoassay (RIA), Western blot, etc. In further examples, such methods may include mass spectrometry analysis methods. Generally, any mass spectrometric (MS) techniques that are capable of obtaining precise information on the mass of peptides, and preferably also on fragmentation and/or (partial) amino acid sequence of selected peptides (e.g., in tandem mass spectrometry, MS/MS; or in post source decay, TOF MS), may be useful herein for separation, detection and/or quantification of proteins. MS peptide analysis methods may be advantageously combined with upstream peptide or protein separation or fractionation methods, such as for example with the chromatographic and other methods. Further techniques for separating, detecting and/or quantifying proteins may be used, optionally in conjunction with any of the above described analysis methods. Such methods include, without limitation, chemical extraction partitioning, isoelectric focusing (IEF) including capillary isoelectric focusing (CIEF), capillary isotachophoresis (CITP), capillary electrochromatography (CEC), and the like, one-dimensional polyacrylamide gel electrophoresis (PAGE), two-dimensional polyacrylamide gel electrophoresis (2D-PAGE), capillary gel electrophoresis (CGE), capillary zone electrophoresis (CZE), micellar electrokinetic chromatography (MEKC), free flow electrophoresis (FFE), etc. In further examples, any combinations of methods such as discussed herein may be employed.

The term “protein” generally encompasses macromolecules comprising one or more polypeptide chains. The term “polypeptide” generally encompasses linear polymeric chains of amino acid residues linked by peptide bonds. A “peptide bond”, “peptide link” or “amide bond” is a covalent bond formed between two amino acids when the carboxyl group of one amino acid reacts with the amino group of the other amino acid, thereby releasing a molecule of water. Especially when a protein is only composed of a single polypeptide chain, the terms “protein” and “polypeptide” may be used interchangeably to denote such a protein. The terms are not limited to any minimum length of the polypeptide chain. Polypeptide chains consisting essentially of or consisting of 50 or less (≤50) amino acids, such as ≤45, ≤40, ≤35, ≤30, ≤25, ≤20, ≤15, ≤10 or ≤5 amino acids may be commonly denoted as a “peptide”. In the context of proteins, polypeptides or peptides, a “sequence” is the order of amino acids in the chain in an amino to carboxyl terminal direction in which residues that neighbour each other in the sequence are contiguous in the primary structure of the protein, polypeptide or peptide. The terms may encompass naturally, recombinantly, semi-synthetically or synthetically produced proteins, polypeptides or peptides. Hence, for example, a protein, polypeptide or peptide can be present in or isolated from nature, e.g., produced or expressed natively or endogenously by a cell or tissue and optionally isolated therefrom; or a protein, polypeptide or peptide can be recombinant, i.e., produced by recombinant DNA technology, and/or can be, partly or entirely, chemically or biochemically synthesised. Without limitation, a protein, polypeptide or peptide can be produced recombinantly by a suitable host or host cell expression system and optionally isolated therefrom (e.g., a suitable bacterial, yeast, fungal, plant or animal host or host cell expression system), or produced recombinantly by cell-free translation or cell-free transcription and translation, or non-biological peptide, polypeptide or protein synthesis. The terms also encompasses proteins, polypeptides or peptides that carry one or more co- or post-expression-type modifications of the polypeptide chain(s), such as, without limitation, glycosylation, lipidation, acetylation, amidation, phosphorylation, sulphonation, methylation, pegylation (covalent attachment of polyethylene glycol typically to the N-terminus or to the side-chain of one or more Lys residues), ubiquitination, sumoylation, cysteinylation, glutathionylation, oxidation of methionine to methionine sulphoxide or methionine sulphone, signal peptide removal, N-terminal Met removal, conversion of pro-enzymes or pre-hormones into active forms, etc. Such co- or post-expression-type modifications may be introduced in vivo by a cell such as a host cell expressing the proteins, polypeptides or peptides (co- or post-translational protein modification machinery may be native to the host cell and/or the host cell may be genetically engineered to comprise one or more (additional) co- or post-translational protein modification functionalities), or may be introduced in vitro by chemical (e.g., pegylation) and/or biochemical (e.g., enzymatic) modification of the isolated proteins, polypeptides or peptides. By means of an example and without limitation, in certain embodiments acetylation of the free alpha amino group at the N-terminus of chemically synthesized peptides and/or the amidation of the free carboxyl group at the C-terminus of chemically synthesized peptides may be opted for to alter the overall charge of the peptides and/or to stabilize the resulting peptides and enhance their ability to resist enzymatic degradation by exopeptidases.

The term “amino acid” encompasses naturally occurring amino acids, naturally encoded amino acids, non-naturally encoded amino acids, non-naturally occurring amino acids, amino acid analogues and amino acid mimetics that function in a manner similar to the naturally occurring amino acids, all in their D- and L-stereoisomers, provided their structure allows such stereoisomeric forms. Amino acids are referred to herein by either their name, their commonly known three letter symbols or by the one-letter symbols recommended by the IUPAC-IUB Biochemical Nomenclature Commission. A “naturally encoded amino acid” refers to an amino acid that is one of the 20 common amino acids or pyrrolysine, pyrroline-carboxy-lysine or selenocysteine. The 20 common amino acids are: Alanine (A or Ala), Cysteine (C or Cys), Aspartic acid (D or Asp), Glutamic acid (E or Glu), Phenylalanine (F or Phe), Glycine (G or Gly), Histidine (H or His), Isoleucine (I or Ile), Lysine (K or Lys), Leucine (L or Leu), Methionine (M or Met), Asparagine (N or Asn), Proline (P or Pro), Glutamine (Q or Gln), Arginine (R or Arg), Serine (S or Ser), Threonine (T or Thr), Valine (V or Val), Tryptophan (W or Trp), and Tyrosine (Y or Tyr). A “non-naturally encoded amino acid” refers to an amino acid that is not one of the 20 common amino acids or pyrrolysine, pyrroline-carboxy-lysine or selenocysteine. The term includes without limitation amino acids that occur by a modification (such as a post-translational modification) of a naturally encoded amino acid, but are not themselves naturally incorporated into a growing polypeptide chain by the translation complex, as exemplified without limitation by N-acetylglucosaminyl-L-serine, N-acetylglucosaminyl-L-threonine, and O-phosphotyrosine. Further examples of non-naturally encoded, un-natural or modified amino acids include 2-Aminoadipic acid, 3-Aminoadipic acid, beta-Alanine, beta-Aminopropionic acid, 2-Aminobutyric acid, 4-Aminobutyric acid, piperidinic acid, 6-Aminocaproic acid, 2-Aminoheptanoic acid, 2-Aminoisobutyric acid, 3-Aminoisobutyric acid, 2-Aminopimelic acid, 2,4 Diaminobutyric acid, Desmosine, 2,2′-Diaminopimelic acid, 2,3-Diaminopropionic acid, N-Ethylglycine, N-Ethylasparagine, homoserine, homocysteine, Hydroxylysine, allo-Hydroxylysine, 3-Hydroxyproline, 4-Hydroxyproline, Isodesmosine, allo-Isoleucine, N-Methylglycine, N-Methylisoleucine, 6-N-Methyllysine, N-Methylvaline, Norvaline, Norleucine, or Ornithine. A further example of such an amino acid is citrulline. Also included are amino acid analogues, in which one or more individual atoms have been replaced either with a different atom, an isotope of the same atom, or with a different functional group. Also included are un-natural amino acids and amino acid analogues described in Ellman et al. Methods Enzymol. 1991, vol. 202, 301-36. The incorporation of non-natural amino acids into proteins, polypeptides or peptides may be advantageous in a number of different ways. For example, D-amino acid-containing proteins, polypeptides or peptides exhibit increased stability in vitro or in vivo compared to L-amino acid-containing counterparts. More specifically, D-amino acid-containing proteins, polypeptides or peptides may be more resistant to endogenous peptidases and proteases, thereby providing improved bioavailability of the molecule and prolonged lifetimes in vivo.

As will be evident from the context, the term “protein” may be recurrently used in this specification to particularly denote the proteins the mutant or variant forms of which are targeted by the molecules as taught herein. In this context, the term may thus provide an expedient reference point in relation to which such variant or mutant forms of the protein can be envisaged and understood. Whereas one can certainly envisage providing variants or mutants of proteins which do not exist in nature, that is of proteins conceived by man, one particularly desirable strength of the present molecules may be the ability to discriminate between naturally occurring proteins and their variants or mutants, preferably their naturally occurring variants or mutants, and to specifically target the latter for downregulation. This may be especially valuable in circumstances where the naturally occurring variants or mutants of the naturally occurring proteins lead to or contribute to some phenotypic detriment, such that their specific downregulation can help to restore normal phenotype underpinned by the maintained expression and activity of the reference protein, or that their specific downregulation can reduce the viability of or kill a cell which has become harmful because of its expressing the mutant or variant protein form.

Accordingly, in certain preferred embodiments, the protein is a naturally occurring protein. In certain preferred embodiments, the protein and the targeted variant or mutant of the protein are naturally occurring. By means of non-limiting examples, the protein may be a naturally occurring protein of a prokaryotic organism, of a eukaryotic organism, or of a virus. For example, the protein may be a naturally occurring protein of an organism belonging to the kingdom Eubacteria, Archaebacteria, Protista, Fungi, Plantae or Animalia. For example, the protein may be a naturally occurring protein of a bacterium, such as more particularly a Gram-positive bacterium (e.g., cocci such as Staphylococcus sp. such as Staphylococcus aureus, Enterococcus sp. such as Enterococcus faecalis or Enterococcus faecium, bacilli such as Bacillus sp. such as Bacillus anthracis), a Gram-negative bacterium (e.g., Escherichia sp. such as Escherichia coli, Yersinia sp. such as Yersinia pestis), a Spirochaetes bacterium (e.g., Treponema sp. such as Treponema pallidum, Leptospira sp. such as Leptospira interrogans, Borrelia sp. such as Borrelia burgdorferi), a Mollicutes bacterium (i.e., a bacterium without a cell wall, such as Mycoplasma sp. such as Mycoplasma pneumoniae or Mycoplasma genitalium), or an acid-fast bacterium (e.g., Mycobacterium sp. such as Mycobacterium tuberculosis, Nocardia sp. such as Nocardia asteroides). For example, the protein may be a naturally occurring protein of a fungus including yeast and moulds (e.g., Candida sp. such as Candida albicans, Aspergillus sp. such as Aspergillus fumigatus or Aspergillus flavus, Coccidioides sp. such as Coccidioides immitis or Coccidioides posadasii, Cryptococcus sp. such as Cryptococcus neoformans and Cryptococcus gattii, Histoplasma sp. such as Histoplasma capsulatum, Pneumocystis sp. such as Pneumocystis jirovecii, or Trichophyton sp. such as Trichophyton mentagrophytes). For example, the protein may be a naturally occurring protein of a protist (e.g., Plasmodium sp. such as Plasmodium falciparum, Entamoeba sp. such as Entamoeba histolytica, Giardia sp. such as Giardia duodenalis, Toxoplasma sp. such as Toxoplasma gondii, Cryptosporidium sp. such as Cryptosporidium parvum, Trichomonas sp. such as Trichomonas vaginalis, Leishmania species such as Leishmania donovani, or Trypanosoma sp. such as Trypanosoma brucei). For example, the protein may be a naturally occurring protein of a plant, e.g., maize, rice, wheat, soybean, barley, sorghum, millet, oat, rye, triticale, buckwheat, quinoa, fonio, einkorn, durum, potato, coffee, cocoa, cassava, tea, rubber tree, coconut palm, oil palm, sugar cane, sugar beet, banana tree, orange tree, pineapple tree, apple tree, pear tree, lemon tree, olive tree, peanut tree, green bean, lettuce, tomato, carrot, zucchini, cauliflower, rapeseed, jatropha, mustard, jojoba, flax, sunflower, green algae, jute, cotton, hemp (or other strains of Cannabis sativa), canola, or tobacco. For example, the protein may be a naturally occurring protein of an animal, preferably a warm-blooded animal, more preferably a vertebrate, yet more preferably a higher animal, still more preferably a mammal, including humans and non-human mammals such as non-human primates, rodents, canines, felines, equines, ovines, or porcines, most preferably a human; such as for example pets (e.g., dogs, cats, rabbits, gerbils, hamsters, chinchillas, mice, rats, guinea pigs, donkeys, mules, ferrets, pygmy goats, pot-bellied pigs; avian pets such as canaries, parakeets, parrots, chickens, turkeys; reptile pets, such as lizards, snakes, tortoises and turtles; aquatic pets, such as fish, frogs), experimental animals (e.g., mice, rats, guinea pigs, rabbits, dogs, pigs, monkeys, ferrets, sheep), livestock animals (e.g., alpaca, banteng, bison, camel, cattle (cows), deer, donkey, gayal, goat, horse, llama, mule, pig, pony, reindeer, sheep, water buffalo, yak). For example, the protein may be a naturally occurring protein of a virus, such as a dsDNA virus (e.g., Adenovirus, Herpesvirus, Poxvirus), ssDNA virus (e.g., Parvovirus), dsRNA virus (e.g., Reovirus), (+)ssRNA virus (e.g., Picornavirus, Togavirus), (−)ssRNA virus (e.g., Orthomyxovirus, Rhabdovirus), ssRNA-RT (reverse transcribing) virus (e.g., Retrovirus), dsDNA-RT virus (e.g., Hepadnavirus), or a bacteriophage.

Due to numerous genome sequencing initiatives over the past decades, the genome sequences of many organisms have been deciphered and the protein encoding genes thereof identified and annotated in public databases such as U.S. government's National Center for Biotechnology Information's (NCBI) Genbank (http://www.ncbi.nlm.nih.gov/) or The UniProt Consortium's Uniprot/Swissprot and Uniprot/TrEMBL databases (http://www.uniprot.org/). Such genome sequencing studies are complemented by plentiful reports on individual proteins, the sequences of which are also annotated in the aforementioned databases. Consequently, the substantially complete protein collections (proteomes) of many organisms are known. Moreover, the number of organisms with sequenced genomes and annotated proteins continues to grow by the day and the tools for genome sequencing and annotation have evolved such as to make them accessible to an average skilled person. Accordingly, the sequences of naturally occurring proteins of many organisms are available or can be readily obtained.

Variant or mutant forms of animal or plant proteins may be particularly interesting objects for the present technology, because such variants or mutants may cause or contribute to phenotypes which deviate from the normal or healthy range of phenotypes of the organism, frequently to the detriment of the organism's well-being or survival. One may therefore wish to alleviate or counter such phenotypes by downregulating the underlying variants or mutants. Downregulating such protein variants or mutants in animals, such as in vertebrates, preferably in higher animals, more preferably in non-human mammals may be particularly useful in animal husbandry or veterinary contexts. Downregulating such protein variants or mutants in humans may be particularly useful in medical contexts. Downregulating such protein variants or mutants in plants may be particularly useful in agricultural or horticultural contexts.

Accordingly, in certain preferred embodiments, the protein is a naturally occurring protein of an animal. In certain preferred embodiments, the protein and the variant or mutant of the protein are naturally occurring animal proteins. In certain preferred embodiments, the protein is a naturally occurring protein of a vertebrate. In certain preferred embodiments, the protein and the variant or mutant of the protein are naturally occurring vertebrate proteins. In certain preferred embodiments, the protein is a naturally occurring protein of a higher animal. In certain preferred embodiments, the protein and the variant or mutant of the protein are naturally occurring higher animal proteins. In certain preferred embodiments, the protein is a naturally occurring protein of a non-human mammal. In certain preferred embodiments, the protein and the variant or mutant of the protein are naturally occurring non-human mammal proteins. Considering the central importance of human health and the need for and value of medical interventions in human subjects, in certain very preferred embodiments, the protein is a naturally occurring human protein. In certain very preferred embodiments, the protein and the variant or mutant of the protein are naturally occurring human proteins. In certain preferred embodiments, the protein is a naturally occurring protein of a plant. In certain preferred embodiments, the protein and the variant or mutant of the protein are naturally occurring plant proteins.

Human genes and proteins are extensively annotated inter alia in the aforementioned Genbank and Uniprot databases. Known variants and mutants (including isoforms, polymorphic forms, disease-causing or associated mutants, etc.) of human proteins are also annotated therein. Human gene nomenclature can further be consulted at the HGNC webpage (https://www.genenames.org/). Additionally, dedicated databases exist which annotate known disease-causing or associated mutations in human genes and proteins. By means of illustration, Online Mendelian Inheritance in Man® (OMIM®, https://www.omim.org/) provides an extensive catalogue of human genes, genetic disorders and the underlying mutations. GWAS Central (https://www.gwascentral.org/) provides a catalogue of associations between unique single nucleotide polymorphisms, which may be in protein-coding sequences, and diseases or phenotypes, as determined by genome-wide association studies (GWAS). Clinical Interpretation of Variants in Cancer database (CIViC, https://civicdb.org/home) provides a database and a forum focused on the clinical significance of cancer genome alterations. The Cancer Genome Atlas (TCGA) Program's GDC data portal (https://portal.gdc.cancer.gov/) collects genomic, epigenomic, transcriptomic, and proteomic data comparing primary cancer and matched normal samples in many cancer types. Catalogue of Somatic Mutations in Cancer (COSMIC, https://cancer.sanger.ac.uk/cosmic) compiles data about somatic mutations in detected in human cancers. The recent publication of the ICGC/TCGA Pan-Cancer Analysis of Whole Genomes Consortium (Nature 2020, vol. 578, 82-93) describes an integrative analysis of 2,658 whole-cancer genomes and their matching normal tissues across 38 tumor types; the associate resources are available via the data portal and visualisations at https://docs.icgc.org/pcawg/.

In certain embodiments, variant or mutant forms of proteins of pathogens may be interesting targets for downregulation, such as particularly where the variation or mutation alters one or more facets of pathogenicity, for example increases or broadens pathogenicity. One may therefore wish to downregulate the underlying variants or mutants to modulate, such as reduce, pathogenicity. The term “pathogen” broadly refers to a biological entity that is pathogenic to a subject, hence, capable of causing a pathological state, condition or disease in the subject, including parasites which can exist in the subject without causing overt disease symptoms. Pathogens encompass viruses, pathogenic microorganisms, such as any pathogenic type of bacteria, protozoa, fungi (including moulds and yeasts), protists (e.g., Plasmodium, Phytophthora, Entamoeba, Giardia, Toxoplasma, Cryptosporidium, Trichomonas, Leishmania, Trypanosoma) (microparasites) and macroparasites such as worms (e.g. nematodes like ascarids, filarias, hookworms, pinworms and whipworms or flatworms like tapeworms and flukes), but also ectoparasites such as ticks and mites. The term also encompasses biological entities, which display pathogenicity in immunocompromised hosts, but may not ordinarily be pathogenic in a non-immunocompromised host. Plant pathogens include without limitation fungi (e.g., Ascomycetes, Basidiomycetes, Oomycetes), bacteria, Phytoplasma, Spiroplasma, viruses, nematodes, protozoa and parasitic plants.

As mentioned above, variants or mutants are discussed herein with respect to “the protein” as a suitable reference point. Preferably the protein and its variants or mutants may be naturally occurring. Sometimes, adjectives such as “unmodified”, “unchanged”, “original”, “starting” may be used in conjunction with the term “the protein” to emphasise the distinction between the protein and its variants or mutants. In certain embodiments, the protein may be the “wild-type” protein in its conventional meaning of the form encoded by the allele of the respective gene that is most commonly observed in a population. In certain embodiments, the protein may be the “wild-type” in protein in its phenotype-oriented meaning of any form that is not causative of or associated with an altered phenotype such as a disease.

With this reference point in mind, the term “variant” (the same can apply to mutants) of a protein may in certain embodiments encompass proteins or polypeptides the amino acid sequence of which is substantially identical (i.e., largely but not wholly identical) to the amino acid sequence of the protein, for example at least about 70% identical, or at least about 75% identical, or at least about 80% identical, or at least about 85% identical, or at least about 90% identical, e.g., at least 91% identical, at least 92% identical, at least 93% identical, at least 94% identical, or at least about 95% identical, e.g., at least 96% identical, at least 97% identical, at least 98% identical, or at least 99% identical. The term “sequence identity” with regard to amino acid sequences denotes the extent of overall sequence identity (i.e., including the whole or entire amino acid sequences in the comparison) expressed in % between the amino acid sequences read from N-terminus to C-terminus. Sequence identity may be determined using suitable algorithms for performing sequence alignments and determination of sequence identity as know per se. Exemplary but non-limiting algorithms include those based on the Basic Local Alignment Search Tool (BLAST) originally described by Altschul et al. 1990 (J Mol Biol 215: 403-10), such as the “Blast 2 sequences” algorithm described by Tatusova and Madden 1999 (FEMS Microbiol Lett 174: 247-250), for example using the published default settings or other suitable settings (such as, e.g., for the BLASTN algorithm: cost to open a gap=5, cost to extend a gap=2, penalty for a mismatch=−2, reward for a match=1, gap x_dropoff=50, expectation value=10.0, word size=28; or for the BLASTP algorithm: matrix=Blosum62 (Henikoff et al., 1992, Proc. Natl. Acad. Sci., 89:10915-10919), cost to open a gap=11, cost to extend a gap=1, expectation value=10.0, word size=3).

An example procedure to determine the percent identity between a particular amino acid sequence and a query amino acid sequence will entail aligning the two amino acid sequences each read from N-terminus to C-terminus using the Blast 2 sequences (B12seq) algorithm, available as a web application or as a standalone executable programme (BLAST version 2.2.31+) at the NCBI web site (www.ncbi.nlm.nih.gov), using suitable algorithm parameters. An example of suitable algorithm parameters includes: matrix=Blosum62, cost to open a gap=11, cost to extend a gap=1, expectation value=10.0, word size=3). If the two compared sequences share identity, then the output will present those regions of identity as aligned sequences. If the two compared sequences do not share identity, then the output will not present aligned sequences. Once aligned, the number of matches will be determined by counting the number of positions where an identical amino acid residue is presented in both sequences. The percent identity is determined by dividing the number of matches by the length of the query sequence, followed by multiplying the resulting value by 100. The percent identity value may, but need not, be rounded to the nearest tenth. For example, 78.11, 78.12, 78.13, and 78.14 may be rounded down to 78.1, while 78.15, 78.16, 78.17, 78.18, and 78.19 may be rounded up to 78.2. It is further noted that the detailed view for each segment of alignment as outputted by B12seq already conveniently includes the percentage of identities.

In certain embodiments, variants may denote different forms of the same protein which arise through alternative splicing of the protein's pre-mRNA. Typically, a splicing variant of a protein may differ from the protein by the presence or absence of one or more contiguous amino acid stretches (encoded by exons) in the variant which are respectively absent or present in the protein, while apart from (or outside of) these stretch or stretches, the sequence of the splicing variant and the protein may be typically identical. Put differently, alternative splicing leads to the inclusion of different combinations of exons in mRNAs made of the same pre-mRNA, whereby the proteins encoded by the mRNAs will differ by the amino acid sequences corresponding to the differentially spliced exons. In such situations, one may talk about splicing variants or isoforms. In certain other embodiments, variants may refer to forms of the protein encoded by distinct alleles of the same gene, where such alleles occur in the natural population, e.g., occur in the natural population at a frequency of 1.0% or more. In such situations, one may talk about allelic variants. In certain further embodiments, variants may refer to forms of the protein encoded by the same mRNA, but wherein amino acid sequence variation arises as a consequence of post-translational modification(s). In certain yet other embodiments, variants may refer to other proteins highly similar (e.g., at least about 70% or more identical as set forth above) to the reference protein and encoded by another gene or locus. In such situations, one may talk about homologues.

The term “mutant” of a protein may in particular denote a form of the protein which differs from the protein in its amino acid sequence, wherein the mutant form is encoded by the same gene or locus as the protein, but wherein the nucleic acid sequence of that gene has been changed such as to encode the mutant form of the protein. Any types of sequence changes are contemplated herein for variants and mutants, including deletions, insertions, and/or substitutions (“deletion” refers to a mutation wherein one or more nucleotides, typically consecutive nucleotides, of a nucleic acid are removed, i.e., deleted, from the nucleic acid; “insertion” refers to a mutation wherein one or more nucleotides, typically consecutive nucleotides, are added, i.e., inserted, into a nucleic acid; “substitution” refers to a mutation wherein one or more nucleotides of a nucleic acid are each independently replaced, i.e., substituted, by another nucleotide). By means of examples, a mutation may result in the deletion, substitution or addition of a single amino acid or of several contiguous amino acids (e.g., 2 to 10 contiguous amino acids) in a protein, without shifting the reading frame for the remainder of the protein. In certain embodiments, a mutation may be a single amino acid substitution, such as a single amino acid substitution modifying an existing APR (e.g., modifying the APR's sequence, TANGO score, and/or length), or leading to the emergence of a de novo APR. Single amino acid substitutions are a mutation type which occurs relatively frequently, single amino acid substitutions in proto-oncogenes or in tumor suppressor genes may contribute to genetic causation of cancer. Or a mutation such as a deletion or addition may shift the reading frame, which may provide the mutated protein with an amino acid sequence not present in the original protein and/or may lead to a premature stop codon and a C-terminally truncated version of the protein. Truncated versions of proteins may frequently display dominant negative effects. Or a mutation in an exon, intron or at an exon-intron boundary may alter the splicing of a protein's pre-mRNA, leading for example to skipping of one or more exons or inclusion of one or more exons, with or without a shift in the reading frame.

Mutations as contemplated herein may also arise in connection with or as a consequence of genetic instability or genomic rearrangements in cells. Such phenomena are particularly commonplace in the case of cancer, including haematological cancers as well as solid tumours, including sarcomas, carcinomas, and CNS tumors, and may also occur in other circumstances or pathological states. Genomic instability can encompass gene mutations, translocations, copy number alterations, deletions, and inversions of pieces of DNA. In certain situations, genomic rearrangements may lead to the formation of fusion genes, containing normally separate genes or parts thereof fused into one. Hence, in certain embodiments, a mutation as contemplated herein may be the formation of a fusion gene encoding a fusion protein. In such embodiments, the mutant form of a protein may be seen as the form in which said protein or a part thereof is fused to another protein or part thereof. Fusion genes were originally discovered in hematologic malignancies but have afterwards been found across solid tumors. Non-limiting examples of fusion genes/proteins found in cancer include BCR-ABL1, EWSR1-FLI1, SS18-SSX1, PML-RARA, EWSR1-ATF1, ETV6-NTRK3, PAX8-PPARG, MECT1-MAML2, TMPRSS2-ERG, TMPRSS2-ETV1, EML4-ALK, KIAA1549-BRAF, MYB-NFIB, ESRRA-C11orf20, FGFR3-TACC3, FGFR3-TACC3, PTPRK-RSPO3, EIF3E3-RSPO2, and SFPQ-TFE3. In the present context, a fusion of a first gene to a second gene, thereby creating a fusion gene encoding a fusion protein, is of particular interest, since the fusion incorporates into the first protein any APRs found in the (fused part of) the second protein/incorporates into the second protein any APRs found in the (fused part of) the first protein; and any such APRs may be deemed de novo APRs present only in the mutant form of the protein, which thus render the mutant protein targetable by the present approach. Also in the present context, novel APRs may emerge or existing APRs may be modified at the precise site of the fusion between the first and second genes, and such APRs, not found in either the first or the second protein, render the fusion protein selectively targetable by the present approach.

Mutant alleles of many genes exist in and are inherited through the germline genetic material of a population. Conventionally, an allele may be deemed a mutant allele rather than a polymorphic variant allele when its frequency in a population is less than 1.0%. Mutations may also occur de novo. Whether pre-existing or arising de novo, mutations are typically the consequence of DNA sequence errors that occur during nucleic acid replication, repair (e.g., repair of DNA damage caused by chemical or physical insults), mitosis or meiosis, or due to insertion of transposons or viral sequences. Many mutations may be silent, such that the mutant protein displays an amino acid sequence difference from the wild-type protein, but without the protein function being perceivably altered. Preferably, mutations as intended herein are not silent, such that some property, function or effect of the protein is affected by the mutation. In certain preferred embodiments, the mutation may be a “gain-of-function” mutation. In certain preferred embodiments, the mutation may be a dominant negative mutation. In preferred embodiments, the mutation, such as the gain-of-function or dominant negative mutation, is detrimental to the functioning or viability of the cell expressing the mutant protein or to the health or fitness of the organism carrying the mutation. In certain embodiments, a mutation may be a germline mutation, i.e., a mutation existing in the germ cells of a parent and passed to the offspring via the gametes produced by that parent, or a mutation arising de novo in the germ cells or gametes of a parent or in the zygote. In certain preferred embodiments, a mutation may be a somatic mutation, i.e., an acquired alteration in DNA of a subject that occurs after conception. Techniques exist to detect somatic mutations in subjects, such as PCR amplification and sequencing or otherwise genotyping a gene in a sample containing somatic cells from a subject, wherein such genetic information may where necessary or informative be compared to the subject's germline sequence variation in that gene. By means of example and without limitation, wherein a somatic mutation is causative of or associated with a neoplastic disease, the presence of the mutation may be determined in samples containing tumor cells of a subject, such as tumor tissue biopsies (e.g., primary or metastatic tumor tissue; e.g., formalin-fixed, paraffin-embedded tumor tissue or fresh-frozen tumour tissue), fine needle aspirates, blood samples (‘liquid’ biopsies), or body exudates into which tumour cells may be shed, such as saliva, urine, stool (feces), tears, sweat, sebum, nipple aspirate, ductal lavage, cerebrospinal fluid, or lymph.

As mentioned above, the variation or mutation as envisaged herein is such that a β-aggregation prone region (APR) existing in the protein is modified by it, or that a new or de novo APR is introduced into the protein by it. For example, a mutant or variant allele that arises in nature may encode a protein with such modified or newly emerged APR.

APRs or self-association regions as used herein denote contiguous amino acid stretches in proteins, which display propensity to self-associate by forming intermolecular beta-sheets. More particularly, APRs as envisaged in this specification encompass regions predicted or defined as such by the statistical mechanics algorithm TANGO (Fernandez-Escamilla et al. Nat Biotechnol. 2004, vol. 22, 1302-6, incorporated by reference herein in its entirety, see specifically Methods section on pages 1305 and 1306 and Supplementary Notes 1 and 2 on the methods and the data sets used for the calibration and the testing of the TANGO algorithm; more background can also be found in WO2007/071789; TANGO algorithm is publically available via http://tango.crg.es/). By means of further explanations, but intending to be in full conformity with the teachings of Fernandez-Escamilla et al. (supra) and http://tango.crg.es/, the model used by the TANGO algorithm is designed to predict beta-aggregation in peptides and proteins and consists of a phase-space encompassing the random coil and the native conformations as well as other major conformational states, namely beta-turn, alpha-helix and beta-aggregate. Every segment of a peptide can populate each of these states according to a Boltzmann distribution. Therefore, to predict self-association regions of a peptide, TANGO calculates the partition function of the phase-space. To estimate the aggregation tendency of a particular amino acid sequence, the following assumptions are made: (i) in an ordered beta-sheet aggregate, the main secondary structure is the beta-strand. (ii) the regions involved in the aggregation process are fully buried, thus paying full solvation costs and gains, full entropy and optimizing their H-bond potential (that is, the number of H-bonds made in the aggregate is related to the number of donor groups that are compensated by acceptors. An excess of donors or acceptors remains unsatisfied). (iii) complementary charges in the selected window establish favourable electrostatic interactions, and overall net charge of the peptide inside but also outside the window disfavours aggregation. The algorithms identifies aggregation prone sequences by comparing the aggregation propensity score of a given amino acid sequence with an average propensity calculated from a set of sequences of similar length.

In certain embodiments, any segment with an aggregation tendency as predicted by TANGO above 5% over 5-6 residues may constitute a potential aggregating segment (APR). Preferably, the aggregation tendency of an APR as intended herein as predicted by TANGO may be ≥6%, ≥7%, ≥8%, ≥9%, preferably ≥10%, ≥15%, more preferably ≥20%, ≥25%, even more preferably ≥30%, ≥40%, or very preferably ≥50%, ≥60%. Preferably, the length of the segment predicted as an APR (not including flanking gatekeeper residues which reduce beta-sheet forming propensity) may be at least 6 contiguous amino acids, preferably between 6 and 16 contiguous amino acids, such as 6, 7, 8, 9, 10, 11, 12, 13, 14, 15 or 16 contiguous amino acids. A high TANGO score of a sequence stretch typically corresponds to a sequence with high (and kinetically favourable) beta-aggregation propensity. By means of an illustrative example, an APR as intended herein as predicted by TANGO may be 6 to 12 contiguous amino acids long and may have TANGO score of >5%, preferably >10%, more preferably >20% or higher.

In certain embodiments, an APR may be constituted by 6 to 16 contiguous amino acids, such as 6, 7, 8, 9, 10, 11, 12, 13, 14, 15 or 16 contiguous amino acids, at least 50% (e.g., ≥55%, ≥60%, ≥65%, preferably ≥70%, ≥75%, more preferably ≥80%, ≥85%, still more preferably ≥90%, ≥95%) of which are hydrophobic amino acids, and in which at least one aliphatic residue or F is present, and if only one aliphatic residue or F is present, at least one, and preferably at least two, other residues are selected from Y, W, A, M and T; and in which no more than 1, and preferably none, P, R, K, D or E residue is present. Hydrophobic amino acids include in particular I, L, V, F, Y, W, H, M, T, K, A, C, and G, preferably I, L, V, F, Y, W, M, T, and A. Aliphatic residues are in particular I, L and V.

Where the variation or mutation modifies an APR which has existed in the original protein, the sequence of the APR may be modified. For example, one or more amino acids of the APR may be substituted; or one or more amino acids may be added to the APR, such as internally or at one or both flanks of the APR; and/or one or more amino acids may be deleted from the APR, such as internally or at one or both flanks of the APR. Such sequence alteration of the APR may but need not modulate the predicted aggregation propensity of the APR, preferably the aggregation propensity of the modified APR may be increased compared to the original APR. In certain embodiments, when the variation or mutation modifies an APR which has existed in the original protein, only the aggregation propensity of the APR may be modified, preferably increased. This may for instance occur when the variation or mutation modifies an amino acid or amino acids proximal to the APR, such as adjacent to the APR, whereby this has an impact on the aggregation propensity of the APR without changing its sequence. Accordingly, in certain embodiments, the APR in the mutant or variant form of the protein differs from the APR in the protein in amino acid sequence. In further embodiments, the APR in the mutant or variant form of the protein differs from the APR in the protein in aggregation propensity. In particularly preferred embodiments, the APR in the mutant or variant form of the protein differs from the APR in the protein in amino acid sequence and aggregation propensity, more preferably increased aggregation propensity. Hence, in certain particularly preferred embodiments, the aggregation propensity of the APR in the mutant or variant form of the protein is higher than the aggregation propensity of the APR in the protein.

Where the variation or mutation introduces into the variant or mutant protein a de novo APR where no corresponding APR has existed in the original protein, this may typically occur when an additional amino acid sequence containing the APR is inserted into the protein, for example by alternative splicing of the protein's pre-mRNA, or by a mutation which alters the splicing pattern of the protein's pre-mRNA, or by an insertion mutation, or by a mutation which causes a frame shift, thereby introducing new sequences into the mutant protein downstream of the mutation, etc. This may also occur when an amino acid stretch that approximates and APR but does not yet qualify as an APR, for example, does not pass the threshold values set by the TANGO algorithm for an APR, is modified by the variation or mutation so that it then does qualify as an APR. For example, one or more amino acids of such proto-APR or pre-APR may be substituted; or one or more amino acids may be added to the proto-APR, such as internally or at one or both flanks of the proto-APR; and/or one or more amino acids may be deleted from the proto-APR, such as internally or at one or both flanks of the proto-APR.

The molecules as taught herein are configured to specifically target the APR in the variant or mutant form of the protein. This may in particular convey that the extent to which a molecule might downregulate the amount or biological activity of the original protein, if at all, is negligible or insignificant compared to the extent to which the molecule downregulates the amount or biological activity of the variant or mutant form of the protein. Where quantifiable assays can be performed to assess the impact of a molecule on the amount or biological activity of a variant or mutant form of the protein vs. the original protein, the reduction in the amount or biological activity produced by the molecule for the original protein may be, in order of increasing preference, at least 10-fold smaller, at least 10²-fold smaller, at least 10³-fold smaller, at least 10⁴-fold smaller, at least 10⁵-fold smaller, or at least 10⁶-fold smaller than the reduction in the amount or biological activity produced by the molecule for the variant or mutant protein. For example: when a cell expressing a variant or mutant form of a protein, wherein the amount or biological activity of the variant or mutant form in the cell can be denoted as 100%, is contacted with an amount of a molecule as taught herein specifically targeted against that variant or mutant form, the amount or biological activity of the variant or mutant form in the cell may be reduced to 50% or less, preferably to 20% or less, more preferably to 10% or less, still more preferably to 1% or less, such as in particularly preferred examples to 0.1%, 0.01%, 0.001% or 0.0001%; on the other hand, when a cell of the same type expressing the protein is contacted with the same amount of the molecule under the same conditions, the cell may retain at least 80%, preferably at least 90%, more preferably at least 95%, still more preferably at least 99% and up to 100% of the amount or biological activity of the protein. In therapeutic context, the specificity of targeting may also mean that the molecules when administered in therapeutically effective and realistic quantities would cause no or only minor or tolerable undesired effects attributable to downregulation of the unmodified protein.

In certain embodiments, the molecule as taught herein is configured to form an intermolecular beta-sheet with the APR in the mutant or variant form of the protein but substantially not with the APR in the original protein (if the original protein contains a corresponding APR).

The terms “beta-sheet”, “beta-pleated sheet”, “p-sheet”, “p-pleated sheet” are well-known in the art and by virtue of additional explanation interchangeably refer to a molecular structure comprising two or more beta-strands connected laterally by backbone hydrogen bonds (interstrand hydrogen bonding). A beta-strand is a stretch of amino acids typically 3 to 10 amino acids long with backbone in an almost fully extended conformation, following a ‘zigzag’ trajectory. Adjacent amino acid chains in a beta-sheet can run in opposite directions (antiparallel β sheet) or in the same direction (parallel β sheet) or may show a mixed arrangement. When not forming a beta-sheet (e.g., prior to participating in a beta-sheet), the stretch of amino acids may exhibit a non-beta-strand conformation; for example it may have an unstructured conformation.

An “intermolecular” beta-sheet involves beta-strands from two or more separate molecules, such as from two or more separate peptides or peptide-containing molecules, polypeptides and/or proteins. In the context of the instant disclosure, the term particularly denotes a beta-sheet involving one or more beta-strands from one or more targeting molecules as taught herein and one or more beta-strands from one or more molecules of the variant or mutant form of the protein. Given that co-aggregation seeded by the intermolecular beta-sheet formation is considered to play an important role in the mode of action of the present molecules, many tens, hundreds, thousands, or more molecules as taught herein and molecules of the variant or mutant form of the protein may be involved in underlying beta-sheets interactions, leading to higher order organisation and structures, such as protofibrils, fibrils and aggregates.

Typically, a beta-strand may be formed by only a part of (e.g., by a stretch of contiguous amino acids of) a molecule, peptide, polypeptide or protein that participates in a beta-sheet. For example, the molecule as taught herein may include one or more stretches of contiguous amino acids which become organised into beta-strands participating in beta-sheets in cooperation with one or more beta-strands constituted by stretches of contiguous amino acids of one or more molecules of the variant or mutant form of the protein. In other words, a statement that a molecule can form and intermolecular beta-sheet with a variant or mutant form of the protein will typically mean that one or more portions of the molecule, such as one or more stretches of contiguous amino acids of the molecule, is or are designed to organise into beta-strands that can participate in a beta-sheet together with one or more stretches of contiguous amino acids, namely one or more APRs, of a variant or mutant form of the protein.

The interlocking of beta-strands from two or more separate molecules into beta sheets can thus create a complex in which the two or more separate molecules become physically associated or connected and spatially adjacent. In view of the aforementioned explanations, the phrase “a molecule configured to form an intermolecular beta-sheet with the APR in the mutant or variant form of the protein” may also subsume the meanings: a molecule capable of participating in or contributing to or inducing the generation of an intermolecular beta-sheet with the APR in the mutant or variant form of the protein; a molecule comprising a portion capable of participating in or contributing to or inducing the generation of an intermolecular beta-sheet with the APR in the mutant or variant form of the protein; and a molecule comprising a stretch of contiguous amino acids capable of participating in or contributing to or inducing the generation of an intermolecular beta-sheet with the APR in the mutant or variant form of the protein.

The characterisation of the present molecules as being able to form an intermolecular beta-sheet with the APR in the mutant or variant form of the protein is based inter alia on the mechanisms described in WO 2007/071789A1 and WO2012/123419A1 as underlying the operation of the ‘interferor’ technology. However, the emergence of beta-sheet conformation may also be experimentally assessed by available methods. By means of a non-limiting example, nuclear magnetic resonance (NMR) spectroscopy has been employed for many years to characterise the secondary structure of proteins in solution (reviewed in Wuetrich et al. FEBS Letters. 1991, vol. 285, 237-247).

Perhaps more straightforwardly in the context of the present invention, the formation of the intermolecular beta-sheet leads to an interaction between the molecule and the mutant or variant form of the protein, which can be qualitatively and quantitatively assessed by standard methods such as co-immunoprecipitation assays. Several instances of such co-immunoprecipitation assays are presented in the Examples for an illustrative mutant form of a wild-type protein, namely human RAS protein mutated at position 12, i.e., G12 mutant human RAS protein. In one illustrative approach, cells expressing G12 mutant or wild-type RAS were contacted with molecules as taught herein labelled with biotin, the cells were lysed, the molecules (and any RAS proteins bound thereto) were pulled down by streptavidin-coated beads, and the co-precipitated RAS protein was quantified by an immunoassay method, namely a quantitative Western blot. In another illustrative approach, in vitro translation reactions producing G12 mutant or wild-type RAS were contacted with molecules as taught herein labelled with biotin, the molecules (and any RAS proteins bound thereto) were pulled down by streptavidin-coated beads, and the co-precipitated RAS protein was quantified by an immunoassay method, namely a quantitative Western blot. Also in the context of the present invention, the interaction between the molecule and the mutant or variant form of the protein can lead to reduced solubility of the mutant or variant form of the protein and even emergence of aggregates or inclusion bodies containing the same. This can be analysed by standard immunoassay or fluorescence microscopy methods also exemplified in the Examples for an illustrative mutant form of a wild-type protein, namely G12 mutant human RAS protein. In one illustrative approach, cells expressing G12 mutant or wild-type RAS were contacted with molecules as taught herein, the cells were lysed by a non-denaturing buffer and proteins insoluble in this buffer were treated with a strong chaotropic agent (6M urea). RAS present in the fraction remaining insoluble after this treatment was quantified by an immunoassay method, namely a quantitative Western blot. In another illustrative approach, cultured mammalian such as human cells were transfected with G12 mutant or wild-type RAS fused to a fluorescent moiety, such as a standard green or red fluorescent protein, the cells were treated with molecules as taught herein and the cellular localization of the fluorescently-tagged RAS was determined by fluorescence microscopy. These illustrative assays, which can be applied and adopted according to circumstances, have the advantage that the molecules can contact the mutant or variant form of the protein when this is being produced on ribosomes (in cells or in vitro). In such not-yet-folded mutant or variant form of the protein the targeted APR is expected to be comparatively more accessible and exposed to the environment, which can facilitate the intermolecular interaction with the molecules. Further in the context of the present invention, the interaction between the molecule and the mutant or variant form of the protein is intended to downregulate the same, which can be detected and quantified for example by measuring the reduction in viability of cells that depend for their growth on the presence of such mutant or variant form of the protein, when exposed to molecules as taught herein. One such exemplary cell line for studying the downregulation of G12 mutant RAS is NCI-H441 lung adenocarcinoma cells, obtainable inter alia from American Type Culture Collection (ATCC) (10801 University Blvd. Manassas, Va. 20110-2209, USA), accession no. HTB-174T′, which depends on constitutive RAS signalling. This is also illustrated in the Examples.

The description of the present molecules as substantially not forming an intermolecular beta-sheet with the APR in the original protein, insofar that protein contains an APR corresponding to that targeted by the molecule, is understandably coterminous with the above discussed specificity of the molecules for targeting the mutant or variant form of the protein, since the selective formation of the intermolecular beta-sheet with the APR in the mutant or variant form of the protein is believed to underlie the specificity of the molecules in targeting the mutant or variant form of the protein. Where assays or tests for detecting the formation of beta-sheets as described above are used, such as in vitro assays or tests performed in cultured cells, e.g., co-immunoprecipitation assays, solubility measurements, or fluorescence microscopy assays to visualise aggregates, the substantial lack of intermolecular beta-sheet formation between the molecules and the unmodified protein may be observed as the absence of a signal (i.e., the absence of an outcome or measurement considered ‘positive’) in the respective assays, or as the presence of a quantifiable signal that is comparable to or not significantly higher than a signal produced by a negative control (e.g., by a molecule of a similar chemical composition but without any or with only negligible beta-sheet forming quality, e.g., by a scrambled peptide in case of peptide molecules), or as the presence of a quantifiable signal that is considerably lower or less intense than the signal produced by the molecule for the mutant or variant form of the protein. For example, the signal (e.g., the quantity of protein co-precipitated with a molecule, the quantity insoluble protein or the proportion of insoluble vs. soluble protein, or the number, size or fluorescence intensity of visible protein aggregates in cells) produced by a molecule for the original protein may be, in order of increasing preference, at least 10-fold lower, at least 10²-fold lower, at least 10³-fold lower, at least 10⁴-fold lower, at least 105-fold lower, or at least 10⁶-fold lower than the signal produced by the molecule for the mutant or variant form of the protein.

Accordingly, the present molecules are designed to induce intermolecular n-sheet formation with their respective target mutant or variant form of a protein, leading to specific downregulation or knock-down thereof. Based on experimental observations, the molecules can bring about reduced solubility and aggregation of the targeted mutant or variant proteins. Hence, in certain embodiments, the molecules as taught herein are able to decrease the solubility or to induce the aggregation or inclusion body formation of the targeted mutant or variant form of the protein. Suitable assays to assess solubility and aggregation of proteins are discussed elsewhere in this specification.

Any meaningful extent of reduction in solubility of the targeted mutant or variant form of the protein is envisaged. This may in appropriate contexts, such as in experimental or therapeutic contexts, denote a statistically significant decrease of the amount of the mutant or variant protein present in the soluble protein fraction, or a statistically significant increase of the amount of the mutant or variant protein present in the insoluble protein fraction, or a statistically significant decrease in the relative abundance of the mutant or variant protein in the soluble vs. insoluble protein fractions, relative to a respective reference. The skilled person is able to select such a reference, such as in particular a reference indicative of the solubility of the mutant or variant protein in the presence of a ‘negative control’ molecule. For example, such decrease in solubility may fall outside of error margins for the reference (as expressed, for example, by standard deviation or standard error, or by a predetermined multiple thereof, e.g., ±1×SD or ±2×SD, or ±1×SE or ±2×SE). By means of an illustration, the solubility of the mutant or variant protein may be considered reduced when it is decreased by at least 10%, such as by at least 20% or by at least 30%, preferably by at least 40%, such as by at least 50% or by at least 60%, more preferably by at least 70%, such as by at least 80% or by at least 90% or more, as compared to the reference, up to and including a 100% decrease (i.e., no mutant or variant protein present in the soluble protein fraction/all mutant or variant protein present in the insoluble protein fraction).

As stated above, beta-strands tend to be 3 to 10 amino acids long. Accordingly, in certain embodiments the intermolecular beta-sheet formed between the molecule and the mutant or variant form of the protein may involve at least 3, such as at least 4 or at least 5, contiguous amino acids of the targeted APR. Put differently, said at least 3, at least 4 or at least 5 contiguous amino acids of the APR will constitute a beta-strand that participates in the beta-sheet. To enhance specificity of the targeting, the molecules may be designed such as to induced beta-sheets that involve at least 6, such as exactly 6, or at least 7, such as exactly 7, or at least 8, such as exactly 8, or at least 9, such as exactly 9, or at least 10, such as exactly 10, contiguous amino acids of the targeted APR. Beta-sheets involving 11, 12, 13 or 14 contiguous amino acids of the APR are also conceivable, even though beta-strands of 6 to 10 contiguous amino acids may be preferred, since they allow for satisfactory specificity while simplifying the design of the molecules.

Further, in certain embodiments, the intermolecular beta-sheet may involve one or more of the amino acids which differ between the mutant or variant form of the protein and the protein. Put differently, the one or more amino acids by which the mutant or variant form of the protein differs from the original protein will be part of a beta-strand that participates in the beta-sheet. This will be particularly so if said one or more amino acids are part of the APR in the mutant or variant form of the protein. Where the mutation or variation results in an APR which includes one or more amino acids which were also present in the original protein, but which in the original protein were not part of an APR, the intermolecular beta-sheet may also involve such one or more amino acids. As an illustration, a G12V mutation in human RAS protein extends an APR predicted in the wild-type human RAS to span positions 2-12, such that the APR in the G12V RAS mutant spans positions 2-15. In such instance, not only the mutated amino acid (V) at position 12, but also the adjacent amino acids at positions 13-15 (GVG) may participate in the beta-sheet formation.

Where the variation or mutation modifies an APR existing in the original protein such that the respective APRs of the mutant or variant and of the original protein differ in their amino acid sequence and/or aggregation propensity, in certain embodiments any one or more of the following may apply:
a) the APR in the mutant or variant form of the protein may have a higher proportion (ratio, percentage) of hydrophobic amino acids than the APR in the protein;
b) the APR in the mutant or variant form of the protein may have a lower proportion of amino acids that display low beta-sheet forming potential or a propensity to disrupt beta-sheets than the APR in the protein;
c) the APR in the mutant or variant form of the protein may have a lower proportion of charged amino acids than the APR in the protein;
d) the APR in the mutant or variant form of the protein may be at least one amino acid longer than the APR in the protein, such as two, three or four amino acids longer.

Such features may also apply when comparing an APR in the mutant or variant form of the protein with a corresponding proto-APR in the unmodified protein.

Hydrophobic amino acids, in particular hydrophobic amino acids other than proline, include V, F, Y, W, H, M, T, K, A, C, and G. Preferably, the hydrophobic amino acid may be I, L, V, F, Y, W, M, T, or A, more preferably I, L, V, F, W, M, and A. By means of an example and without limitation, where the APR in the protein comprises at least 50% or at least 60% or at least 70% hydrophobic amino acids, the APR in the mutant or variant form of the protein may comprise more than 50% (e.g., 60% or more or 70% or more), more than 60% (e.g., 70% or more or 80% or more) or more than 70% (e.g., 80% or more or 90% or more) hydrophobic amino acids, respectively. In certain embodiments, the APR in the mutant or variant form of the protein may have a higher proportion of aliphatic amino acids, in particular I, L and/or V, or F than the APR in the protein.

An amino acid having low beta-sheet forming potential or propensity to disrupt beta-sheets may be R, K, E, D, P, N, S, H, G or Q. An amino acid having a particularly low beta-sheet forming potential or a particularly high propensity to disrupt beta-sheets may be a charged amino acid, such as R, K, D or E, or an amino acid typified by high conformational rigidity, in particular P. By means of an example and without limitation, where the APR in the protein comprises 3, 2 or 1 amino acids having low beta-sheet forming potential or propensity to disrupt beta-sheets, the APR in the mutant or variant form of the protein may comprise 2, 1 or 0, 1 or 0, or 0 such amino acids, respectively.

Charged amino acids in proteins include R, K, H, E, and D, and may preferably refer to R, K, E or D. By means of an example and without limitation, where the APR in the protein comprises 3, 2 or 1 charged amino acids, the APR in the mutant or variant form of the protein may comprise 2, 1 or 0, 1 or 0, or 0 such amino acids, respectively.

The mutation or variation may also affect the length of the APR, and may preferably increase the length of the APR, such as by one, two, three or four amino acids. By means of an example and without limitation, where the APR in the protein is 6 or 8 or 10 contiguous amino acids long, the APR in the mutant or variant form of the protein may be more than 6 (e.g., 7 to 16), more than 8 (e.g., 9 to 16) or more than 10 (e.g., 11 to 16) amino acids long.

In view of the foregoing explanations, in certain embodiments any one or more of the following may apply:
a) the mutation or variation in the mutant or variant form of the protein may modify, such as substitute, delete or add, one or more amino acids within the APR in the protein;
b) the mutation or variation in the mutant or variant form of the protein may modify, such as substitute, delete or add, one or more amino acids within a region of between 1 and 10, preferably between 1 and 4 contiguous amino acids N-terminally adjacent to the APR in the protein, preferably whereby at least one amino acid of said region becomes part of the APR in the mutant or variant form of the protein;
c) the mutation or variation in the mutant or variant form of the protein may modify, such as substitute, delete or add, one or more amino acids within a region of between 1 and 10, preferably between 1 and 4 contiguous amino acids C-terminally adjacent to the APR in the protein, preferably whereby at least one amino acid of said region becomes part of the APR in the mutant or variant form of the protein.

Put differently, the mutation or variation may affect the sequence of the contiguous amino acid stretch which was predicted to constitute an APR in the unmodified protein, such as without limitation one or more amino acids of said stretch (e.g., non-hydrophobic amino acids, such as polar or charged amino acids) may be substituted with one or more other amino acids (e.g., hydrophobic amino acids). Or the mutation or variation may affect the sequences which N-terminally and/or C-terminally flank or enclose the APR in the unmodified protein. Typically, in native proteins, APRs are flanked by amino acids that display comparatively lower beta-sheet forming potential or a propensity to disrupt beta-sheets (e.g., as predicted by TANGO or as discussed above). The inclusion of such ‘gatekeeper’ sequences serves to control the aggregation propensity of APRs in native properties, thereby minimising or avoiding self-aggregation in conditions of normal expression in cells. Typically, such flanking gatekeeper regions may each independently span 1-10, more typically 1-6, even more typically 1-4, such as 1, 2, 3 or 4 contiguous amino acids N-terminally and C-terminally adjacent to the APR. Accordingly, a mutation or variation in such flanking regions may alter the characteristics of these regions, such that the APR in the mutant or variant form of the protein extends or projects into what was previously a flanking or gatekeeper region. Without limitation, this may occur when one or more non-hydrophobic or less hydrophobic amino acids of an APR-flanking region is substituted by one or more (more) hydrophobic amino acids, such as one or more aliphatic amino acids.

Hence, in certain embodiments, the mutation or variation in said region N- or C-terminally adjacent to the APR in the protein may:
a) increase the proportion of hydrophobic amino acids in said region;
b) reduce the proportion of amino acids that display low beta-sheet forming potential or a propensity to disrupt beta-sheets said region; and/or
c) reduce the proportion of charged amino acids in said region.

The present molecules are able to induce the formation of an intermolecular beta-sheet with a mutant or variant form of a protein. To this end, the molecules may advantageously comprise at least one portion that can assume or mimic a beta-strand conformation capable of interacting with the beta-strand contributed by the mutant or variant protein, more particularly by its APR, so as to give rise to an intermolecular beta-sheet formed by said interacting beta-strands.

In certain embodiments, the molecule may comprise at least one amino acid stretch which participates in the intermolecular beta-sheet with the APR in the mutant or variant form of the protein. As explained earlier, beta-strands tend to be 3 to 10 amino acids long. Accordingly, in certain embodiments the at least one amino acid stretch comprised by the molecule may be at least 3, such as at least 4 or at least 5, contiguous amino acids long. To enhance specificity of the interaction, the at least one amino acid stretch comprised by the molecule may be at least 6, such as exactly 6, or at least 7, such as exactly 7, or at least 8, such as exactly 8, or at least 9, such as exactly 9, or at least 10, such as exactly 10, contiguous amino acids long. Amino acid stretches that are 11, 12, 13 or 14 contiguous amino acids long can also be conceivably comprised by the molecule, but stretches of 6 to 10 contiguous amino acids may be preferred, since they allow for satisfactory specificity while simplifying the design of the molecules. Accordingly, in certain embodiments the molecule comprises an amino acid stretch of at least 6 contiguous amino acids which participates in the intermolecular beta-sheet. In further embodiments, the molecule comprises an amino acid stretch of 6 to 10 contiguous amino acids which participates in the intermolecular beta-sheet.

In certain preferred embodiments, the at least one stretch of amino acids, such as the at least one stretch of at least 6 contiguous amino acids or of 6 to 10 contiguous amino acids, comprised by the molecule (henceforth “the molecule stretch” for brevity) may correspond to the stretch of contiguous amino acids comprised by the APR in the mutant or variant form of the protein which is to participate in the beta-sheet (henceforth “the mutant/variant stretch” for brevity). By means of certain examples, when the beta-sheet is to involve a mutant/variant stretch of 3, 4, 5, preferably 6 to 10, such as 6, 7, 8, 9 or 10, or even 11, 12, 13 or 14 contiguous amino acids of the APR, the molecule stretch can correspond to this mutant/variant stretch.

The correspondence between the molecule stretch and the mutant/variant stretch may in particular encompass:
a) the situation that the amino acid sequence of the molecule stretch is identical to the amino acid sequence of the mutant/variant stretch;
b) the situation that the amino acid sequence of the molecule stretch is at least 80% identical to the amino acid sequence of the mutant/variant stretch, insofar this degree of sequence identity is compatible with the formation of the intermolecular beta-sheet as taught herein—for example, said at least 80% sequence identity may in certain embodiments denote that when the mutant/variant stretch is 6 or 7 amino acids long the 6 or 7 amino acid-long molecule stretch differs from the mutant/variant stretch by at most 1 amino acid substitution, or when the mutant/variant stretch is 8 to 12 amino acids long the 8 to 12 amino acid-long molecule stretch differs from the mutant/variant stretch by at most 2 amino acid substitutions, or when the mutant/variant stretch is 13 to 14 amino acids long the 13 to 14 amino acid-long molecule stretch differs from the mutant/variant stretch by at most 3 amino acid substitutions;
c) the situation that the amino acid sequence of the molecule stretch differs from the amino acid sequence of the mutant/variant stretch by at most 3, preferably at most 2, and more preferably at most 1 amino acid substitutions, insofar this substitution or substitutions are compatible with the formation of the intermolecular beta-sheet as taught herein;
d) the situation that the amino acid sequence of the molecule stretch displays the degree of sequence identity to the amino acid sequence of the mutant/variant stretch as set forth in any one of a) to c) above, and all amino acids of the molecule stretch are L-amino acids;
e) the situation that the amino acid sequence of the molecule stretch displays the degree of sequence identity to the amino acid sequence of the mutant/variant stretch as set forth in any one of a) to c) above, and at least one (e.g., at least 2, at least 3, at least 4, at least 5, or at least 6 or more or all) amino acid of the molecule stretch is a D-amino acid, insofar the incorporation of the D-amino acid or D-amino acids is compatible with the formation of the intermolecular beta-sheet as taught herein;
f) the situation that the amino acid sequence of the molecule stretch displays the degree of sequence identity to the amino acid sequence of the mutant/variant stretch as set forth in any one of a) to c) above, and at least one (e.g., at least 2, at least 3, at least 4, at least 5, or at least 6 or more or all) amino acid of the molecule stretch is replaced by an analogue of the respective amino acid, insofar the incorporation of the analogue or analogues is compatible with the formation of the intermolecular beta-sheet as taught herein; or
g) the situation that the amino acid sequence of the molecule stretch displays the degree of sequence identity to the amino acid sequence of the mutant/variant stretch as set forth in any one of a) to c) above, and at least one amino acid of the molecule stretch is a D-amino acid and at least one amino acid of the molecule stretch is replaced by an analogue of the respective amino acid, insofar the incorporation of the D-amino acid or D-amino acids and the analogue or analogues is compatible with the formation of the intermolecular beta-sheet as taught herein.

Preferably, the molecule stretch may be designed such that its amino acid sequence is not identical to an amino acid sequence in proteins of the respective organism (such as human organism where a human mutant or variant protein is targeted) other than the mutant or variant protein, to reduce or prevent off-target activity of molecules containing such molecule stretch. The amino acid sequence of the molecule stretch can be readily aligned with the full proteome of the organism to perform this assessment.

As mentioned, in certain embodiments the amino acid sequence of the molecule stretch may be less than 100% identical to the amino acid sequence of the mutant/variant stretch, for example, the molecule stretch sequence may be at least 80%, e.g., 81%, 82%, 83%, or 84%, preferably at least 85%, e.g., 86%, 87%, 88%, or 89%, more preferably at least 90%, e.g., 91%, 92%, 93%, 94%, 95%, 96%, 97%, 98%, or 99%, identical to the mutant/variant stretch sequence.

In such embodiments, the molecule stretch may comprise one or more amino acid additions, deletions, or substitutions relative to (i.e., compared with) the mutant/variant stretch. Preferably, the molecule stretch may comprise one or more amino acid substitutions, preferably at most 3 or more preferably at most 2 or even more preferably at most 1 amino acid substitution, such as in particular one or more single amino acid substitutions, preferably at most 3 or more preferably at most 2 or even more preferably at most 1 single amino acid substitution, relative to the mutant/variant stretch.

Preferably, the one or more amino acid substitutions, in particular the one or more single amino acid substitutions may be conservative amino acid substitutions. A conservative amino acid substitution is a substitution of one amino acid for another with similar characteristics. Conservative amino acid substitutions include substitutions within the following groups: valine, alanine and glycine; leucine, valine, and isoleucine; aspartic acid and glutamic acid; asparagine and glutamine; serine, cysteine, and threonine; lysine and arginine; and phenylalanine and tyrosine. The nonpolar hydrophobic amino acids include alanine, leucine, isoleucine, valine, proline, phenylalanine, tryptophan and methionine. The polar neutral amino acids include glycine, serine, threonine, cysteine, tyrosine, asparagine and glutamine. The positively charged (i.e., basic) amino acids include arginine, lysine and histidine. The negatively charged (i.e., acidic) amino acids include aspartic acid and glutamic acid. Any substitution of one member of the above-mentioned polar, basic, or acidic groups by another member of the same group can be deemed a conservative substitution. By contrast, a non-conservative substitution is a substitution of one amino acid for another with dissimilar characteristics.

In certain embodiments, the one or more amino acid substitutions, in particular the one or more single amino acid substitutions, may each independently be with an uncharged amino acid, preferably with a hydrophobic amino acid other than proline, such as with glycine (G), alanine (A), valine (V), leucine (L), isoleucine (I), phenylalanine (F), methionine (M), and tryptophan (W). Such substitutions can increase the beta-sheet inducing potential of the molecule stretch.

In certain preferred embodiments, the amino acid or amino acids of the molecule stretch that correspond to or align with the mutated or variant amino acid or amino acids in the targeted mutant or variant protein may be identical to, or may be a D-isomer of or may be an analogue of, preferably are identical to, said mutated or variant amino acid(s).

Further, as illustrated above, the molecule stretch, i.e., the at least one amino acid stretch comprised by the molecules as taught herein which participates in the intermolecular beta-sheet, may also include D-amino acids and/or analogues of the recited amino acids. Stated more generally, in certain embodiments, the at least one amino acid stretch of the molecule may comprise one or more D-amino acids, or analogues of one or more of its amino acids, or one or more D-amino acids and analogues of one or more of its amino acids, provided the incorporation of the D-amino acid or D-amino acids and/or the analogue or analogues is compatible with the formation of the intermolecular beta-sheet as taught herein.

Without limitation, in certain embodiments the molecule stretch may include only one D-amino acid. In certain embodiments, the molecule stretch may include two or more (e.g., 3, 4, 5, 6 or more) D-amino acids. In certain embodiments, about 10%, about 20%, about 30%, about 40%, about 50%, about 60%, about 70%, about 80%, about 90%, or 100% (i.e., all) amino acids constituting the molecule stretch may be D-amino acids. In certain embodiments, the D-amino acids may be interspersed between L-amino acids and/or the D-amino acids may be organised into one or more sub-stretches of two or more D-amino acids separated by L-amino acids. Without limitation, in certain embodiments the molecule stretch may include an analogue of only one of its amino acids. In certain embodiments, the molecule stretch may include analogues of two or more (e.g., 3, 4, 5, 6 or more) of its amino acids. In certain embodiments, the molecule stretch may include analogues of about 10%, about 20%, about 30%, about 40%, about 50%, about 60%, about 70%, about 80%, about 90%, or 100% (i.e., all) of its amino acids. In certain embodiments, the amino acid analogues may be interspersed between naturally occurring amino acids and/or the amino acid analogues may be organised into one or more sub-stretches of two or more such analogues separated by naturally occurring amino acids. Without limitation, in certain embodiments the molecule stretch may include only one constituent that is a D-amino acid or a amino acid analogue. In certain embodiments, the molecule stretch may include two or more (e.g., 3, 4, 5, 6 or more) constituents that are D-amino acids or amino acid analogues. In certain embodiments, about 10%, about 20%, about 30%, about 40%, about 50%, about 60%, about 70%, about 80%, about 90%, or 100% (i.e., all) constituents of the molecule stretch may be D-amino acids or amino acid analogues.

As already explained, the molecule stretch may be designed to correspond to the mutant/variant stretch, which may in particular call for a certain degree of sequence identity between the molecule stretch and the mutant/variant stretch. For example, the molecule stretch may be most preferably identical to the mutant/variant stretch, or may differ from the latter only by single amino acid substitution(s), in particular by no more than 3, preferably no more than 2, more preferably no more than 1 single amino acid substitutions. Such comparatively high extent of sequence identity between the molecule stretch and the mutant/variant stretch aims to allow the stretches to associate, in particular through the formation of an intermolecular beta-sheet there between. It has indeed been reported that ‘self-association’ of beta-aggregating regions within naturally occurring proteins is a widespread underlying mechanism of aggregation of such proteins (see for example Fernandez-Escamilla et al. 2004, supra), and the present approach is able to take advantage of this. As also already explained, the notion of correspondence between a molecule stretch and a mutant/variant stretch does allow for the inclusion of D-isomers and/or analogues of the respective amino acids in the molecule stretch.

The reference to an amino acid analogue may encompass any compound that has the same or similar basic chemical structure as a naturally-encoded amino acid, i.e., an organic compound comprising a carboxyl group, an amino group, and an R moiety (amino acid residue). Typically, the amino group and the R moiety may be bound to the α carbon atom (i.e., the carbon atom to which the carboxyl group is bound). In other embodiments, the amino group may be bound to α carbon atom other than the α carbon atom, for example, to the β or γ carbon atom, preferably to the β carbon atom. In such embodiments, the R moiety may be bound to the same carbon atom as the amino group or to α carbon atom closer to the α carbon atom or to the α carbon atom itself. Typically, where the carboxyl group, the amino group and the R moiety are bound to the α carbon atom, the α carbon atom may also be bound to a hydrogen atom. Typically, where the amino group and the R moiety are bound to the β carbon atom, the β carbon atom may also be bound to a hydrogen atom. Without limitation, the R moiety of an amino acid analogue may differ from the R group of the respective naturally-encoded amino acid by one or more individual atoms or functional groups of the R group being replaced or substituted with a different atom (e.g., a methyl group replaced with a hydrogen atom, or an S atom replaced with an O atom, etc.), with an isotope of the same atom (e.g., ¹²C replaced with ¹³C, ¹⁴N replaced with ¹⁵N, or ¹H replaced with ²H, etc.), or with a different functional group (e.g., a hydrogen atom replaced with a methyl, ethyl or propyl group, or with another alkyl, alkenyl, cycloalkyl, cycloalkenyl, heterocyclyl, aryl, or heteroaryl group; an —SH group replaced with an —OH group or —NH₂ group, etc.). The structural difference or modification in an amino acid analogue compared to the respective naturally-encoded amino acid preferably preserves the core property of the amino acid with respect to charge and polarity. Hence, an amino acid analogue of a non-polar hydrophobic amino acid may preferably also have a non-polar hydrophobic R moiety; an amino acid analogue of a polar neutral amino acid may preferably also have a polar neutral R moiety; an amino acid analogue of a positively charged (basic) amino acid may preferably also have a positively charged R moiety, preferably with the same number of charged groups; and an amino acid analogue of a negatively charged (acidic) amino acid may preferably also have negatively charged R moiety, preferably with the same number of charged groups. All amino acid analogues are envisaged as both D- and L-stereoisomers, provided their structure allows such stereoisomeric forms.

By means of an example and without limitation, a leucine analogue may be selected from the list consisting of 2-amino-3,3-dimethyl-butyric acid (t-Leucine), alpha-methylleucine, hydroxyleucine, 2,3-dehydro-leucine, N-alpha-methyl-leucine, 2-Amino-5-methyl-hexanoic acid (homoleucine), 3-Amino-5-methylhexanoic acid (beta-homoleucine), 2-Amino-4,4-dimethyl-pentanoic acid (4-methyl-leucine, neopentylglycine), 4,5-dehydro-norleucine, L-norleucine, N-alpha-methyl-norleucine, and 6-hydroxy-norleucine, including their D- and L-stereoisomers, provided their structure allows such stereoisomeric forms. By means of an example and without limitation, a valine analogue may be selected from the list consisting of c-alpha-methyl-valine (2,3-dimethylbutanoic acid), 2,3-dehydro-valine, 3,4-dehydro-valine, 3-methyl-L-isovaline (methylvaline), 2-amino-3-hydroxy-3-methylbutanoic acid (hydroxyvaline), beta-homovaline, and N-alpha-methyl-valine, including their D-and L-stereoisomers, provided their structure allows such stereoisomeric forms. By means of an example and without limitation, a glycine analogue may be selected from the list consisting of N-alpha-methyl-glycine (sarcosine), cyclopropylglycine, and cyclopentylglycine, including their D- and L-stereoisomers, provided their structure allows such stereoisomeric forms. By means of an example and without limitation, an alanine analogue may be selected from the list consisting of 2-amino-isobutyric acid (2-methylalanine), 2-amino-2-methylbutanoic acid (isovaline), N-alpha-methyl-alanine, c-alpha-methyl-alanine, c-alpha-ethyl-alanine, 2-amino-2-methylpent-4-enoic acid (alpha-allylalanine), beta-homoalanine, 2-indanyl-glycine, di-n-propyl-glycine, di-n-butyl-glycine, diethylglycine, (1-naphthyl)alanine, (2-naphthyl)alanine, cyclohexylglycine, cyclopropylglycine, cyclopentylglycine, adamantyl-glycine, and beta-homoallylglycine, including their D- and L-stereoisomers, provided their structure allows such stereoisomeric forms.

In certain embodiments, the molecule may comprise exactly one amino acid stretch which participates in the intermolecular beta-sheet (i.e., exactly one ‘molecule stretch’ as discussed above). In certain preferred embodiments, the molecule may comprise two or more amino acid stretches which participate in the intermolecular beta-sheet (i.e., two or more ‘molecule stretches’ as discussed above). For example, the molecule may comprise 2 to 6, preferably 2 to 5, more preferably 2 to 4, or even more preferably 2 or 3 molecule stretches. For example, the molecule may comprise exactly 2, or exactly 3, or exactly 4, or exactly 5 molecule stretches, particularly preferably exactly 2 or exactly 3 molecule stretches, even more preferably exactly 2 molecule stretches. The inclusion of two or more molecule stretches tends to increase the effectiveness of the molecules in downregulating and inducing aggregation of the respective mutant or variant proteins. Hence, in preferred embodiments, the two or more molecule stretches will be directed to the same mutant or variant protein. However, a configuration where the two or more molecule stretches are directed to different mutant or variant proteins can be envisaged, and can provide for a more universal targeting agent.

Where the molecule comprises two or more molecule stretches as taught herein, these may each independently be identical or different. For example, in a molecule with exactly 2 molecule stretches, the 2 molecule stretches may be identical or different; in a molecule with exactly 3 molecule stretches, all 3 stretches may be identical, or each stretch may be different from each other stretch, or 2 stretches may be identical and the remaining stretch may be different; or in a molecule with exactly 4 molecule stretches, all 4 stretches may be identical, or each stretch may be different from each other stretch, or 2 or 3 stretches may be identical and the remaining stretch(es) may be different from the former and optionally identical to each other.

By means of examples and without limitation, where two molecule stretches are said to be different, each molecule stretch may correspond to a different mutant/variant stretch as taught herein, such as for example to non-overlapping, overlapping, or nested, but nonetheless different, mutant/variant stretches, preferably of the same mutant or variant protein. In such embodiments, the two molecule stretches may be designed with different underlying amino acid sequences in mind, and may optionally also differ in other respects such as in the extent to which they incorporate (or not) amino acid substitutions, D-isomers and/or analogues of the respective amino acids. Or where two molecule stretches are said to be different, each molecule stretch may correspond to the same mutant/variant stretch, such that the two molecule stretches are designed with the same underlying amino acid sequence in mind, but can differ in other respects such as in the extent to which they incorporate (or not) amino acid substitutions, D-isomers and/or analogues of the respective amino acids. In particularly preferred embodiments, the two or more molecule stretches correspond to the same mutant/variant stretch, more preferably the two or more molecule stretches do not differ in amino acid substitutions (e.g., they might not incorporate any amino acid substitutions compared to the mutant/variant stretch or may incorporate the same amino acid substitutions), and even more preferably also do not differ in the extent to which they incorporate D-isomers and/or analogues of the respective amino acids (e.g., they might not incorporate any D-isomers and/or analogues or may incorporate the same D-isomers and/or analogues at the same position(s)). Hence, in particularly preferred embodiments, the two or more molecule stretches are identical.

Where the molecule comprises two or more amino acid stretches which participate in the intermolecular beta-sheet (i.e., two or more ‘molecule stretches’ as discussed above), the reference to “the intermolecular beta-sheet” does not necessarily denote physically the same beta-sheet, but may denote another beta-sheet with another mutant or variant protein molecule. For example, a molecule with two molecule stretches may engage two mutant or variant protein molecules in the same beta-sheet, or in two separate beta-sheets, or initially in two separate beta-sheets which later become part of the same beta-sheet or the same higher order structure driven by beta-sheet formation. Hence, what is particularly sought is the occurrence of conformational changes in the targeted APR of the mutant or variant protein molecules towards beta-strands and beta-sheets, which eventually decreases solubility and causes aggregation thereof.

In preferred embodiments, to reduce the propensity of the molecules containing the above-discussed amino acid stretch or stretches to self-associate or self-aggregate even before being exposed to their target mutant or variant protein (e.g., to precipitate upon production or during storage), the amino acid stretch or stretches may be enclosed or gated by amino acids that can reduce or prevent such self-association (also termed “gatekeeper amino acids” or “gatekeepers”). Accordingly, in certain embodiments, the amino acid stretch or stretches within the molecule are each independently flanked, in particular directly or immediately flanked, on each end independently, by one or more amino acids, in particular contiguous amino acids, that display low beta-sheet forming potential or a propensity to disrupt beta-sheets. Typically, such flanking regions may each independently comprise 1 to 10, preferably 1 to 8, more preferably 1 to 6, or even more preferably 1 to 4, such as exactly 1, exactly 2, exactly 3 or exactly 4 amino acids, particularly contiguous amino acids, that have low beta-sheet forming potential or propensity to disrupt beta-sheets.

In certain preferred embodiments, an amino acid having low beta-sheet forming potential or propensity to disrupt beta-sheets may be a charged amino acid, such as a positively charged (basic, such as overall +1 or +2 charge) amino acid or a negatively charged (acidic, such as overall −1 or −2 charge) amino acid, such as an amino acid containing an amino group (—NH₃⁺ when protonated) or a carboxyl group (—COO— when dissociated) in its R moiety. In certain other embodiments, an amino acid having low beta-sheet forming potential or propensity to disrupt beta-sheets may be an amino acid typified by high conformational rigidity, for example due to the inclusion of its peptide bond-forming amino group in a heterocycle, such as in pyrrolidine.

Hence, in certain preferred embodiments, an amino acid having low beta-sheet forming potential or propensity to disrupt beta-sheets may be R, K, E, D, P, N, S, H, G, Q, or A, including D- and L-stereoisomers thereof, or analogues thereof. In certain preferred embodiments, an amino acid having low beta-sheet forming potential or propensity to disrupt beta-sheets may be R, K, E, D, P, N, S, H, G or Q, including D- and L-stereoisomers thereof, or analogues thereof. In certain more preferred embodiments, an amino acid having low beta-sheet forming potential or propensity to disrupt beta-sheets may be R, K, E, D or P, including D- and L-stereoisomers thereof, or analogues thereof. In certain more preferred embodiments, an amino acid having low beta-sheet forming potential or propensity to disrupt beta-sheets may be R, K, E or D, including D- and L-stereoisomers thereof, or analogues thereof. Accordingly, in certain embodiments, the amino acid stretch or stretches within the molecule are each independently flanked, on each end independently, by one or more amino acids, preferably by 1 to 4 contiguous amino acids, selected from the group consisting of R, K, E, D, P, N, S, H, G, Q, and A, D- and L-stereoisomers thereof, and analogues thereof, and combinations thereof; or selected from the group consisting of R, K, E, D, P, N, S, H, G, and Q, D- and L-stereoisomers thereof, and analogues thereof, and combinations thereof; or selected from the group consisting of R, K, E, D, and P, D- and L-stereoisomers thereof, and analogues thereof, and combinations thereof.

By means of an example and without limitation, an arginine analogue, in particular an arginine analogue that carries a positive charge or can be protonated to carry a positive charge, may be selected from the list consisting of 2-amino-3-ureido-propionic acid, norarginine, 2-amino-3-guanidino-propionic acid, glyoxal-hydroimidazolone, methylglyoxal-hydroimidazolone, N′-nitro-arginine, homoarginine, omega-methyl-arginine, N-alpha-methyl-arginine, N,N′-diethyl-homoarginine, canavanine, and beta-homoarginine, including their D- and L-stereoisomers, provided their structure allows such stereoisomeric forms. By means of an example and without limitation, a lysine analogue, in particular a lysine analogue that carries a positive charge or can be protonated to carry a positive charge, may be selected from the list consisting of N-epsilon-formyl-lysine, N-epsilon-methyl-lysine, N-epsilon-1-propyl-lysine, N-epsilon-dimethyl-lysine, N-epsilon-trimethylamonium-lysine, N-epsilon-nicotinyl-lysine, ornithine, N-delta-methyl-ornithine, N-delta-N-delta-dimethyl-ornithine, N-delta-1-propyl-ornithine, c-alpha-methyl-ornithine, beta,beta-dimethyl-ornithine, N-delta-methyl-N-delta-butyl-ornithine, N-delta-methyl-N-delta-phenyl-ornithine, c-alpha-methyl-lysine, beta,beta-dimethyl-lysine, N-alpha-methyl-lysine, homolysine, and beta-homolysine, including their D- and L-stereoisomers, provided their structure allows such stereoisomeric forms. By means of an example and without limitation, a glutamic or aspartic acid analogue, in particular a glutamic or aspartic acid analogue that carries a negative charge or can dissociate to carry a negative charge, may be selected from the list consisting of 2-amino-adipic acid (homoglutamic acid), 2-amino-heptanedioic acid (2-aminopimelic acid), 2-amino-octanedioic acid (aminosuberic acid), and 2-amino-4-carboxy-pentanedioic acid (4-carboxyglutamic acid), including their D- and L-stereoisomers, provided their structure allows such stereoisomeric forms.

By means of an example and without limitation, a proline analogue may be selected from the list consisting of 3-methylproline, 3,4-dehydro-proline, 2-[(2S)-2-(hydrazinecarbonyl)pyrrolidin-1-yl]-2-oxoacetic acid, beta-homoproline, alpha-methyl-proline, hydroxyproline, 4-oxo-proline, beta,beta-dimethyl-proline, 5,5-dimethyl-proline, 4-cyclohexyl-proline, 4-phenyl-proline, 3-phenyl-proline, and 4-aminoproline, including their D- and L-stereoisomers, provided their structure allows such stereoisomeric forms. A further non-limiting example of an amino acid that may be included in a gatekeeper moiety or moieties as disclosed herein, possibly in combination with other amino acids, is diaminopimelic acid. A further non-limiting example of an amino acid that may be included in a gatekeeper moiety or moieties as disclosed herein, possibly in combination with other amino acids, is citrulline.

By means an illustration and without limitation, examples of such gatekeeper sequences or regions that can flank the molecule stretches may be, each independently, R, K, E, D, P, A, diaminopimelic acid, citrulline, RR, KK, EE, DD, PP, RK, KR, ED, DE, RRR, KKK, DDD, EEE, PPP, RRK, RKK, KKR, KRR, RKR, KRK, DDE, DEE, EED, EDD, EDE, or DED, etc., wherein any arginine, lysine, glutamate, aspartate, proline, or alanine may be L- or D-isomer, and optionally wherein any arginine, lysine, glutamate, aspartate, proline, or alanine may be substituted by its analogue as discussed elsewhere in this specification.

As discussed earlier, the molecules can comprise at least one portion that can assume or mimic a beta-strand conformation capable of interacting with the beta-strand contributed by the mutant or variant protein APR so as to give rise to an intermolecular beta-sheet formed by said interacting beta-strands, while in certain embodiments, such portion may preferably be an amino acid stretch (‘molecule stretch’) which participates in the intermolecular beta-sheet. In certain other embodiments, the portion may be a peptidomimetic of such a molecule stretch. The term “peptidomimetic” refers to a non-peptide agent that is a topological analogue of a corresponding peptide. Methods of rationally designing peptidomimetics of peptides are known in the art. For example, the rational design of three peptidomimetics based on the sulphated 8-mer peptide CCK26-33, and of two peptidomimetics based on the 11-mer peptide Substance P, and related peptidomimetic design principles, are described in Horwell 1995 (Trends Biotechnol 13: 132-134).

The chemical nature and structure of the molecules outside of the portions that are intended to interlock with the beta-strands of the mutant or variant protein APR, such as in other words outside of the ‘molecule stretch or stretches’ as discussed hitherto, is comparatively less critical, insofar these remaining sections or portions of the molecule do not interfere with or preferably facilitate or enable the aforementioned intermolecular beta-sheet interaction.

In certain embodiments, where the molecule comprises two or more molecule stretches as discussed herein, each optionally and preferably flanked by gatekeeper regions, these molecule stretches are connected, in particular covalently connected, directly or preferably through a linker (also known as spacer). The incorporation of such linkers or spacers may endow the individual molecule stretches with more conformational freedom and less steric hindrance to interact with the mutant or variant protein. Optionally, in addition to being interposed between the molecule stretches, linkers may also be added outside of the first and/or outside of the last molecule stretch of the molecule. This applies mutatis mutandis for molecules only including one molecule stretch, optionally and preferably flanked by gatekeeper regions, wherein linkers may be coupled to one or both ends of the single molecule stretch.

The nature and structure of such linkers is not particularly limited. The linker may be a rigid linker or a flexible linker. In particular embodiments, the linker is a covalent linker, achieving a covalent bond. The terms “covalent” or “covalent bond” refer to a chemical bond that involves the sharing of one or more electron pairs between two atoms. A linker may be, for example, a (poly)peptide or non-peptide linker, such as a non-peptide polymer, such as a non-biological polymer. Preferably, any linkages may be hydrolytically stable linkages, i.e., substantially stable in water at useful pH values, including in particular under physiological conditions, for an extended period of time, e.g., for days.

In certain embodiments, each linker may be independently selected from a stretch of between 1 and 20 identical or non-identical units, wherein a unit is an amino acid, a monosaccharide, a nucleotide or a monomer. Non-identical units can be non-identical units of the same nature (e.g. different amino acids, or some copolymers). They can also be non-identical units of a different nature, e.g. a linker with amino acid and nucleotide units, or a heteropolymer (copolymer) comprising two or more different monomeric species. According to specific embodiments, each linker may be independently composed of 1 to 10 units of the same nature, particularly of 1 to 5 units of the same nature. According to particular embodiments, all linkers present in the molecule may be of the same nature, or may be identical.

In particular embodiments, any one linker may be a peptide or polypeptide linker of one or more amino acids. In certain embodiments, all linkers in the molecule may be peptide or polypeptide linkers. More particularly, the peptide linker may be 1 to 20 amino acids long, such as preferably 1 to 10 amino acids long, such as more preferably 2 to 5 amino acids long. For example, the linker may be exactly 1, 2, 3, 4, 5, 6, 7, 8, 9, or 10 amino acids long, such as preferably exactly 2, 3 or 4 amino acids long. The nature of amino acids constituting the linker is not of particular relevance so long as the biological activity of the molecule stretches linked thereby is not substantially impaired. Preferred linkers are essentially non-immunogenic and/or not prone to proteolytic cleavage. In certain embodiments, the linker may contain a predicted secondary structure such as an alpha-helical structure. However, linkers predicted to assume flexible, random coil structures are preferred. Linkers having tendency to form beta-strands may be less preferred or may need to be avoided. Cysteine residues may be less preferred or may need to be avoided due to their capacity to form intermolecular disulphide bridges. Basic or acidic amino acid residues, such as arginine, lysine, histidine, aspartic acid and glutamic acid may be less preferred or may need to be avoided due to their capacity for unintended electrostatic interactions. In certain preferred embodiments, the peptide linker may comprise, consist essentially of or consist of amino acids selected from the group consisting of glycine, serine, alanine, phenylalanine, threonine, proline, and combinations thereof, including D-isomers and analogues thereof. In certain preferred embodiments, the peptide linker may comprise, consist essentially of or consist of amino acids selected from the group consisting of glycine, serine, alanine, threonine, proline, and combinations thereof, including D-isomers and analogues thereof. In even more preferred embodiments, the peptide linker may comprise, consist essentially of or consist of amino acids selected from the group consisting of glycine, serine, and combinations thereof, including D-isomers and analogues thereof. In certain embodiments, the peptide linker may consist of only glycine and serine residues. In certain embodiments, the peptide linker may consist of only glycine residues or analogues thereof, preferably of only glycine residues. In certain embodiments, the peptide linker may consist of only serine residues or D-isomers or analogues thereof, preferably of only serine residues. Such linkers provide for particularly good flexibility. In certain embodiments, the linker may consist essentially of or consist of glycine and serine residues. In certain embodiments, the glycine and serine residues may be present at a ratio between 4:1 and 1:4 (by number), such as about 3:1, about 2:1, about 1:1, about 1:2 or about 1:3 glycine:serine. Preferably, glycine may be more abundant than serine, e.g., a ratio between 4:1 and 1.5:1 glycine:serine, such as about 3:1 or about 2:1 glycine:serine (by number). In certain embodiments, the N-terminal and C-terminal residues of the linker are both a serine residue; or the N-terminal and C-terminal residues of the linker are both glycine residues; or the N-terminal residue is a serine residue and the C-terminal residue is a glycine residue; or the N-terminal residue is a glycine residue and the C-terminal residue is a serine residue. In certain embodiments, the peptide linker may consist of only proline residues or D-isomers or analogues thereof, preferably of only proline residues. By means of examples and without limitation, peptide linkers as intended herein may be e.g. PP, PPP, GS, SG, SGG, SSG, GSS, GGS, GSGS (SEQ ID NO: 70), AS, SA, GF, FF, etc.

In certain embodiments, the linker may be a non-peptide linker. In preferred embodiments, the non-peptide linker may comprise, consist essentially of or consist of a non-peptide polymer. The term “non-peptide polymer” as used herein refers to a biocompatible polymer including two or more repeating units linked to each other by a covalent bond excluding the peptide bond. For example, the non-peptide polymer may be 2 to 200 units long or 2 to 100 units long or 2 to 50 units long or 2 to 45 units long or 2 to 40 units long or 2 to 35 units long or 2 to 30 units long or 5 to 25 units long or 5 to 20 units long or 5 to 15 units long. The non-peptide polymer may be selected from the group consisting of polyethylene glycol, polypropylene glycol, copolymers of ethylene glycol and propylene glycol, polyoxyethylated polyols, polyvinyl alcohol, polysaccharides, dextran, polyvinyl ethyl ether, biodegradable polymers such as PLA (poly(lactic acid) and PLGA (polylactic-glycolic acid), lipid polymers, chitins, hyaluronic acid, and combinations thereof. Particularly preferred is poly(ethylene glycol) (PEG). Another particularly envisaged chemical linker is Ttds (4,7,10-trioxatridecan-13-succinamic acid). The molecular weight of the non-peptide polymer preferably may range from 1 to 100 kDa, and preferably 1 to 20 kDa. The non-peptide polymer may be one polymer or a combination of different types of polymers. The non-peptide polymer has reactive groups capable of binding to the elements which are to be coupled by the linker. Preferably, the non-peptide polymer has a reactive group at each end. Preferably, the reactive group is selected from the group consisting of a reactive aldehyde group, a propione aldehyde group, a butyl aldehyde group, a maleimide group and a succinimide derivative. The succinimide derivative may be succinimidyl propionate, hydroxy succinimidyl, succinimidyl carboxymethyl or succinimidyl carbonate. The reactive groups at both ends of the non-peptide polymer may be the same or different. In certain embodiments, the non-peptide polymer has a reactive aldehyde group at both ends. For example, the non-peptide polymer may possess a maleimide group at one end and, at the other end, an aldehyde group, a propionic aldehyde group or a butyl aldehyde group. When a polyethylene glycol (PEG) having a reactive hydroxy group at both ends thereof is used as the non-peptide polymer, the hydroxy group may be activated to various reactive groups by known chemical reactions, or a PEG having a commercially-available modified reactive group may be used so as to prepare the protein conjugate.

In certain particularly preferred embodiments, the operative part of the molecule, i.e., the part responsible for the effects on the mutant or variant protein, may be a peptide. Put differently, in such embodiments, the molecule stretch or stretches that form beta-strands interacting with the mutant or variant protein APR, the optional and preferred flanking gatekeeper regions, the linkers optionally and preferably interposed between the molecule stretches, and the linkers optionally but less preferably added outside of the outermost molecule stretches, are all composed of amino acids (which may include D- and L-stereoisomers and amino acid analogues) covalently linked by peptide bonds. Preferably, the total length of such peptide operative part of the molecule does not exceed 50 amino acids, such as does not exceed 45, 40, 35, 30, 25 or even 20 amino acids. Such peptide operative part of the molecule may be coupled to one or more other moieties, which themselves may but need not be amino acids, peptides, or polypeptides, and which may serve other functions, such as allowing to detect the molecule, increasing the half-life of the molecule when administered to subjects, increasing the solubility of the molecule, increasing the cellular uptake of the molecule, etc., as discussed elsewhere in this specification. In certain particularly preferred embodiments, the molecule is a peptide. Preferably, the total length of such peptide does not exceed 50 amino acids, such as does not exceed 45, 40, 35, 30, 25 or even 20 amino acids. Where the molecule comprises, consists essentially of or consists of, e.g., is, a peptide the N-terminus of said molecule can be modified, such as for example by acetylation, and/or the C-terminus of said molecule can be modified, such as for example by amidation.

In view of the foregoing discussion, in certain embodiments, the molecule as taught herein may be conveniently represented as comprising, consisting essentially of or consisting of the structure:
a) NGK1-P1-CGK1,
b) NGK1-P1-CGK1-Z1-NGK2-P2-CGK2,
c) NGK1-P1-CGK1-Z1-NGK2-P2-CGK2-Z2-NGK3-P3-CGK3, or
d) NGK1-P1-CGK1-Z1-NGK2-P2-CGK2-Z2-NGK3-P3-CGK3-Z3-NGK4-P4-CGK4,
wherein:
P1 to P4 each independently denote the amino acid stretch (‘molecule stretch’) as taught above,
NGK1 to NGK4 and CGK1 to CGK4 each independently denote the gatekeeper region as taught above, and
Z1 to Z3 each independently denote a direct bond or preferably the linker as taught above.

Hence, structure a) refers to a molecule only containing one molecule stretch as taught herein, while structures b), c) and d) refer to molecules containing two, three or four molecule stretch as taught herein, respectively.

In certain embodiments, as explained above, NGK1 to NGK4 and CGK1 to CGK4 may each independently denote 1 to 4 contiguous amino acids that display low beta-sheet forming potential or a propensity to disrupt beta-sheets, such as 1 to 4 contiguous amino acids selected from the group consisting of R, K, D, E, P, N, S, H, G, Q, and A, D-isomers and/or analogues thereof, and combinations thereof, preferably 1 to 4 contiguous amino acids selected from the group consisting of R, K, D, E, P, N, S, H, G, and Q, D-isomers and/or analogues thereof, and combinations thereof, more preferably 1 to 4 contiguous amino acids selected from the group consisting of R, K, D, E, and P, D-isomers and/or analogues thereof, and combinations thereof. In certain embodiments, NGK1 to NGK4 and CGK1 to CGK4 may each independently denote 1 to 2 contiguous amino acids selected from the group consisting of R, K, A, and D, D-isomers and/or analogues thereof, and combinations thereof, such as NGK1 to NGK4 and CGK1 to CGK4 may be each independently K, R, D, A, or KK. In certain particularly preferred embodiments, NGK1 to NGK4 and CGK1 to CGK4 may each independently denote 1 to 2 contiguous amino acids selected from the group consisting of R, K, and D, D-isomers and/or analogues thereof, and combinations thereof, such as NGK1 to NGK4 and CGK1 to CGK4 may be each independently K, R, D or KK.

In certain particularly preferred embodiments, each linker is independently selected from a stretch of between 1 and 10 units, preferably between 1 and 5 units, wherein a unit is each independently an amino acid or PEG, such as each linker is independently GS, PP, AS, SA, GF, FF, or GSGS (SEQ ID NO: 70), or D-isomers and/or analogues thereof, preferably each linker is independently GS, PP or GSGS (SEQ ID NO: 70), preferably GS, or D-isomers and/or analogues thereof. In certain preferred embodiments, each independently, a direct bond is included instead of a linker.

In certain preferred embodiments, the molecule comprises, consists essentially of or consists of a peptide of the structure:
a) Gate-Pept-Gate;
b) Linker-Gate-Pept-Gate;
c) Gate-Pept-Gate-Linker;
d) Linker-Gate-Pept-Gate-Linker;
e) Gate-Pept-Gate-(Linker)-Gate-Pept-Gate;
f) Linker-Gate-Pept-Gate-(Linker)-Gate-Pept-Gate;
g) Gate-Pept-Gate-(Linker)-Gate-Pept-Gate-Linker;
h) Linker-Gate-Pept-Gate-(Linker)-Gate-Pept-Gate-Linker;
i) Gate-Pept-Gate-(Linker)-Gate-Pept-Gate-(Linker)-Gate-Pept-Gate;
j) Linker-Gate-Pept-Gate-(Linker)-Gate-Pept-Gate-(Linker)-Gate-Pept-Gate;
k) Gate-Pept-Gate-(Linker)-Gate-Pept-Gate-(Linker)-Gate-Pept-Gate-Linker; or
l) Linker-Gate-Pept-Gate-(Linker)-Gate-Pept-Gate-(Linker)-Gate-Pept-Gate-Linker;
wherein “Gate”, “Pept”, and “Linker” denote peptide elements bound to the adjacent peptide element(s) by peptide bond(s), wherein left-to-right order of the peptide elements signifies their N- to C-terminal organisation in the peptide;
wherein “Pept” each independently denote the amino acid stretch (‘molecule stretch’) as taught above;
wherein “Gate” is each independently lysine (K) or D-lysine or D- or L-lysine analogue (preferably lysine), arginine (R) or D-arginine or D- or L-arginine analogue (preferably arginine), aspartic acid (D) or D-aspartic acid or D- or L-aspartic acid analogue (preferably aspartic acid), glutamic acid (E) or D-glutamic acid or D- or L-glutamic acid analogue (preferably glutamic acid), KK, KKK, KKKK (SEQ ID NO: 45), RR, RRR, RRRR (SEQ ID NO: 46), DD, DDD, DDDD (SEQ ID NO: 47), EE, EEE, EEEE (SEQ ID NO: 48), KR, RK, KKR, KRK, RKK, RRK, RKR, KRR, KRKR (SEQ ID NO: 49), KRRK (SEQ ID NO: 50), RKKR (SEQ ID NO: 51), DE, ED, DDE, DED, EED, EED, EDE, DEE, DEDE (SEQ ID NO: 52), DEED (SEQ ID NO: 53), or EDDE (SEQ ID NO: 54), optionally wherein any one or more or all of the recited amino acids is or are replaced by its or their D-isomer(s) or by its or their analogue(s), including L- and D-isomers of such analogue(s); and wherein the inclusion of the word “Linker” in parentheses denotes that the linker, each independently, may be absent or is preferably present, and wherein “Linker” is each independently glycine (G) or D- or L-glycine analogue (preferably glycine), serine (S) or D-serine or D- or L-serine analogue (preferably serine), proline (P) or D-proline or D- or L-proline analogue (preferably proline), GG, GGG, GGGG (SEQ ID NO: 55), SS, SSS, SSSS (SEQ ID NO: 56), GS, SG, GGS, GSG, SGG, SSG, SGS, SSG, GGGS (SEQ ID NO: 57), GGSG (SEQ ID NO: 58), GSGG (SEQ ID NO: 59), SGGG (SEQ ID NO: 60), GGSS (SEQ ID NO: 61), GSSG (SEQ ID NO: 62), SSGG (SEQ ID NO: 63), GSGS (SEQ ID NO: 70), SGSG (SEQ ID NO: 64), GSGSG (SEQ ID NO: 65), SGSGS (SEQ ID NO: 66), PP, PPP, or PPPP (SEQ ID NO: 67), optionally wherein any one or more or all of the recited amino acids is or are replaced by its or their D-isomer(s) or by its or their analogue(s), including L- and D-isomers of such analogue(s).

In such peptides, the N-terminal amino acid may be modified such as acetylated and/or the C-terminal amino acid may be modified such as amidated. In such peptides, D-amino acid(s) and or amino acid analogue(s) can be incorporated as long as their incorporation is compatible with the formation of the intermolecular beta-sheet as taught herein.

As already touched upon above, in certain embodiments, the molecule as taught herein may comprise one or more further moieties, groups, components or parts, which may serve other functions or perform other roles and activities. Such functions, roles or activities may be useful or desired for example in connection with the production, synthesis, isolation, purification or formulation of the molecule, or in connection with its in experimental or therapeutic uses. Conveniently, the operative part of the molecule, i.e., the part responsible for the effects on the mutant or variant protein, may be connected to one or more such further moieties, groups, components or parts, preferably covalently connected, bound, linked or fused, directly or through a linker. Where such further moiety, group, component or part is a peptide, polypeptide or protein, the connection to the operative part of the molecule may preferably involve a peptide bond, direct one or through a peptide linker.

For all such added moieties, the nature of the fusion or linker is not vital to the invention, as long as the moiety and the molecule can exert their specific function. According to particular embodiments, the moieties which are fused to the molecules can be cleaved off, e.g. by using a linker moiety that has a protease recognition site. This way, the function of the moiety and the molecule can be separated, which may be particularly interesting for larger moieties, or for embodiments where the moiety is no longer necessary after a specific point in time, e.g., a tag that is cleaved off after a separation step using the tag.

In certain preferred embodiments, the molecule may comprise a detectable label, a moiety that allows for isolation of the molecule, a moiety increasing the stability of the molecule, a moiety increasing the solubility of the molecule, a moiety increasing the cellular uptake of the molecule, a moiety effecting targeting of the molecule to cells, or a combination of any two or more thereof. It shall be appreciated that a single moiety can carry out two or more functions or activities.

Hence, in certain embodiments the molecule may comprise a detectable label. The term “label” refers to any atom, molecule, moiety or biomolecule that may be used to provide a detectable and preferably quantifiable read-out or property, and that may be attached to or made part of an entity of interest, such as molecules as taught herein, such as peptides as taught herein. Labels may be suitably detectable by for example mass spectrometric, spectroscopic, optical, colourimetric, magnetic, photochemical, biochemical, immunochemical or chemical means. Labels include without limitation dyes; radiolabels such as isotopes of hydrogen, carbon, nitrogen, oxygen, phosphorous, sulphur, fluorine, chlorine, or iodine, such as ²H, ³H, ¹³C, ¹¹C, ¹⁴C, ¹⁵N, ¹⁸O, ¹⁷O, ³¹P, ³²P, ³³P, ³⁵S, ¹⁸F, ³⁶Cl, ¹²⁵I, or ¹³¹I respectively; electron-dense reagents; enzymes (e.g., horse-radish peroxidase or alkaline phosphatase as commonly used in immunoassays); binding moieties such as biotin-streptavidin; haptens such as digoxigenin; luminogenic, phosphorescent or fluorogenic moieties; mass tags; fluorescent dyes (e.g., fluorophores such as fluorescein, carboxyfluorescein (FAM), tetrachloro-fluorescein, TAMRA, ROX, Cy3, Cy3.5, Cy5, Cy5.5, Texas Red, etc.) alone or in combination with moieties that may suppress or shift emission spectra by fluorescence resonance energy transfer (FRET); and fluorescent proteins (e.g., GFP, RFP). Certain isotopically labelled molecules such as peptides as taught herein, for example those into which radioactive isotopes such as ³H and ¹⁴C are incorporated, are useful in drug and/or substrate tissue distribution assays. ³H and ¹⁴C isotopes are particularly preferred for their ease of preparation and detectability. Further, substitution with heavier isotopes such as 2H may afford certain therapeutic advantages resulting from greater metabolic stability, for example increased in vivo half-life or reduced dosage requirements and, hence, may be preferred in some circumstances. Isotopically labelled molecules such as peptides may generally be prepared by carrying production or synthesis methods in which a readily available isotopically labelled reagent is substituted for a non-isotopically labelled reagent. In some embodiments, the molecule may be provided with a tag that permits detection with another agent (e.g., with a probe binding partner). Such tags may be, for example, biotin, streptavidin, his-tag, myc tag, FLAG tag (DYKDDDDK, SEQ ID NO: 68), maltose, maltose binding protein or any other kind of tag known in the art that has a binding partner. Example of associations which may be utilised in the probe:binding partner arrangement may be any, and includes, for example biotin:streptavidin, his-tag:metal ion (e.g., Ni²⁺), maltose:maltose binding protein, etc. Labelled mutant or variant-targeting molecules can lend themselves to a variety of uses and applications, such as without limitation, uses in in vitro assays, including diagnostic assays, where the labelled pept-ins may provide a principle which binds to and allows for detection of the respective mutant or variant proteins of interest in a biological sample from a subject; or use in in vivo imaging, where distribution of the labelled mutant or variant-targeting pept-ins in the body may be followed by non-invasive imaging methods after administrations.

In further embodiments, the molecule may comprise a moiety that allows for the isolation (separation, purification) of the molecule. Typically, such moieties operate in conjunction with affinity purification methods, in which the ability to isolate a particular component of interest from other components is conferred by specific binding between a separable binding agent, such as an immunological binding agent (antibody), and the component of interest. Such affinity purification methods include without limitation affinity chromatography and magnetic particle separation. Such moieties are well-known in the art and non-limiting examples include biotin (isolatable using an affinity purification method utilising streptavidin), his-tag (isolatable using an affinity purification method utilising metal ion, e.g., Ni²⁺), maltose (isolatable using an affinity purification method utilising maltose binding protein), glutathione S-transferase (GST) (isolatable using an affinity purification method utilising glutathione), or myc or FLAG tag (isolatable using an affinity purification method utilising anti-myc or anti-FLAG antibody, respectively).

In further embodiments, the molecule may comprise a moiety that increases the solubility of the molecule. While the solubility of the molecules can be ensured and controlled by the inclusion of gatekeeper portions flanking the molecule stretch or stretches as discussed above, whereby this may in principle be sufficient to prevent premature aggregation of the molecules and keep them in solution, the further addition of a moiety that increases solubility, i.e., prevents aggregation, may provide easier handling of the molecules, and particularly improve their stability and shelf-life. Many of the labels and isolation tags discussed above will also increase the solubility of the molecule. Further, a well-known example of such solubilising moiety is PEG (polyethylene glycol). This moiety is particularly envisaged, as it can be used as linker as well as solubilising moiety. Other examples include peptides and proteins or protein domains, or even whole proteins, e.g. GFP. In this regard, it should be noted that, like PEG, one moiety can have different functions or effects. For instance, a FLAG tag is a peptide moiety that can be used as a label, but due to its charge density, it will also enhance solubilisation. PEGylation has already often been demonstrated to increase solubility of biopharmaceuticals (e.g., Veronese and Mero, BioDrugs. 2008; 22(5):315-29). Adding a peptide, polypeptide, protein or protein domain tag to a molecule of interest has been extensively described in the art. Examples include, but are not limited to, peptides derived from synuclein (e.g., Park et al., Protein Eng. Des. Sel. 2004; 17:251-260), SET (solubility enhancing tag, Zhang et al., Protein Expr Purif 2004; 36:207-216), thioredoxin (TRX), Glutathione-S-transferase (GST), Maltose-binding protein (MBP), N-Utilization substance (NusA), small ubiquitin-like modifier (SUMO), ubiquitin (Ub), disulfide bond C (DsbC), Seventeen kilodalton protein (Skp), Phage T7 protein kinase fragment (T7PK), Protein G B1 domain, Protein A IgG ZZ repeat domain, and bacterial immunoglobulin binding domains (Hutt et al., J Biol Chem.; 287(7):4462-9, 2012). The nature of the tag will depend on the application, as can be determined by the skilled person. For instance, for transgenic expression of the molecules described herein, it might be envisaged to fuse the molecules to a larger domain to prevent premature degradation by the cellular machinery. Other applications may envisage fusion to a smaller solubilisation tag (e.g., less than 30 amino acids, or less than 20 amino acids, or even less than 10 amino acids) in order not to alter the properties of the molecules too much.

In further embodiments, the molecule may comprise a moiety increasing the stability of the molecule, e.g., the shelf-life of the molecule, and/or the half-life of the molecule, which may involve increasing the stability of the molecule and/or reducing the clearance of the molecule when administered. Such moieties may modulate pharmacokinetic and pharmacodynamic properties of the molecule. Many of the labels, isolation tags and solubilisation tags discussed above will also increase the shelf-life or in vivo half-life of the molecules, and the inclusion of D-amino acids and/or amino acid analogues may do so as well. For instance, it is known that fusion with albumin (e.g., human serum albumin), albumin-binding domain or a synthetic albumin-binding peptide improves pharmacokinetics and pharmacodynamics of different therapeutic proteins (Langenheim and Chen, Endocrinol.; 203(3):375-87, 2009). Another moiety that is often used is a fragment crystallizable region (Fc) of an antibody. Strohl (BioDrugs. 2015, vol. 29, 215-39) reviews fusion protein-based strategies for half-life extension of biologics, including without limitation fusion to human IgG Fc domain, fusion to HSA, fusion to human transferrin, fusion to artificial gelatin-like protein (GLP), etc. In particular embodiments, the molecules are not fused to an agarose bead, a latex bead, a cellulose bead, a magnetic bead, a silica bead, a polyacrylamide bead, a microsphere, a glass bead or any solid support (e.g. polystyrene, plastic, nitrocellulose membrane, glass), or the NusA protein. However, these fusions are possible, and in specific embodiments, they are also envisaged.

In further embodiments, the molecule may comprise a moiety that increases the cellular uptake of the molecule. For example, the molecules can further comprise a sequence which mediates cell penetration (or cell translocation), i.e., the molecules are further modified through the recombinant or synthetic attachment of a cell penetration sequence. Cell-penetrating peptides (CPP) or protein transduction domain (PTD) sequences are well known in the art. The terms generally refer to peptides capable of entering into cells. This ability can be exploited for the delivery of molecules as disclosed herein to cells. Exemplary but non-limiting CPP include HIV-1 Tat-derived CPP (see, e.g., Frankel et al. 1988 (Science 240: 70-73)); Antennapedia peptides or penetratins (see, e.g., Derossi et al. 1994 (J Biol Chem 269: 10444-10450)); peptides derived from HSV-1 VP22 (see, e.g., Aints et al. 2001 (Gene Ther 8: 1051-1056)); transportans (see, e.g., Pooga et al. 1998 (FASEB J 12: 67-77)); protegrin 1 (PG-1) anti-microbial peptide SynB (Kokryakov et al. 1993 (FEBS Lett 327: 231-236)); model amphipathic (MAP) peptides (see, e.g., Oehlke et al. 1998 (Biochim Biophys Acta 1414: 127-139)); signal sequence-based cell-penetrating peptides (NLS) (see, e.g., Lin et al. 1995 (J Biol Chem 270: 14255-14258)); hydrophobic membrane translocating sequence (MTS) peptides (see, e.g., Lin et al. 1995, supra); and polyarginine, oligoarginine and arginine-rich peptides (see, e.g., Futaki et al. 2001 (J Biol Chem 276: 5836-5840)). Still other commonly used cell-permeable peptides (both natural and artificial peptides) are disclosed e.g. in Sawant and Torchilin, Mol Biosyst. 6(4):628-40, 2010; Noguchi et al., Cell Transplant. 19(6):649-54, 2010 and Lindgren and Langel, Methods Mol Biol. 683:3-19, 2011. The carrier peptides that have been derived from these proteins show little sequence homology with each other, but are all highly cationic and arginine or lysine rich. CPP can be of any length. For example CPP may be less than or equal to 500, 250, 150, 100, 50, 25, 10 or 6 amino acids in length. For example CPP may be greater than or equal to 4, 5, 6, 10, 25, 50, 100, 150 or 250 amino acids in length. Preferably, a CPP may be between 4 and 25 amino acids in length. The suitable length and design of the CPP will be easily determined by those skilled in the art. As a general reference on CPPs can serve inter alia “Cell penetrating peptides: processes and applications” (ed. Ulo Langel, 1st ed., CRC Press 2002); Advanced Drug Delivery Reviews 57: 489-660 (2005); Dietz & Bahr 2004 (Moll Cell Neurosci 27: 85-131)). An agent as disclosed herein may be conjugated with a CPP directly or indirectly, e.g., by means of a suitable linker, such as without limitation a PEG-based linker. Molecules described herein might not need a CPP to enter a cell. Indeed, as is shown in the examples, it is possible to target intracellular proteins, which require that the molecules are taken up by the cell, and this happens without fusion to a CPP.

In further embodiments, the molecule may comprise a moiety effecting targeting of the molecule to cells. For instance, the molecule may be fused to, e.g., an antibody, a peptide or a small molecule with a specificity for a given target, in particular with specificity to a cell expressing the mutant or variant protein to which the molecule is directed, with specificity to a protein specifically expressed on the surface of that cell. In such embodiments, the molecule initiates downregulation or aggregation of the mutant or variant protein specifically in the targeted cells. In certain cases a binding domain is a chemical compound (e.g. a small compound with an affinity for at least one target protein) and in certain other cases a binding domain is a polypeptide, in certain other cases a binding domain is a protein domain. A protein binding domain is an element of overall protein structure that is self-stabilizing and often folds independently of the rest of the protein chain. Binding domains vary in length from between about 25 amino acids up to 500 amino acids and more. Many binding domains can be classified into folds and are recognizable, identifiable, 3-D structures. Some folds are so common in many different proteins that they are given special names. Non-limiting examples are Rossman folds, TIM barrels, armadillo repeats, leucine zippers, cadherin domains, death effector domains, immunoglobulin-like domains, phosphotyrosine-binding domain, pleckstrin homology domain, src homology 2 domain, the BRCT domain of BRCA1, G-protein binding domains, the Eps 15 homology (EH) domain and the protein-binding domain of p53. Antibodies are the natural prototype of specifically binding proteins with specificity mediated through hypervariable loop regions, so called complementary determining regions (CDR).

As used herein, the term “antibody” is used in its broadest sense and generally refers to any immunologic binding agent. The term specifically encompasses intact monoclonal antibodies, polyclonal antibodies, multivalent (e.g., 2-, 3- or more-valent) and/or multi-specific antibodies (e.g., bi- or more-specific antibodies) formed from at least two intact antibodies, and antibody fragments insofar they exhibit the desired biological activity (particularly, ability to specifically bind an antigen of interest, i.e., antigen-binding fragments), as well as multivalent and/or multi-specific composites of such fragments. The term “antibody” is not only inclusive of antibodies generated by methods comprising immunisation, but also includes any polypeptide, e.g., a recombinantly expressed polypeptide, which is made to encompass at least one complementarity-determining region (CDR) capable of specifically binding to an epitope on an antigen of interest. Hence, the term applies to such molecules regardless whether they are produced in vitro or in vivo.

An antibody may be any of IgA, IgD, IgE, IgG and IgM classes, and preferably IgG class antibody. An antibody may be a polyclonal antibody, e.g., an antiserum or immunoglobulins purified there from (e.g., affinity-purified). An antibody may be a monoclonal antibody or a mixture of monoclonal antibodies. Monoclonal antibodies can target a particular antigen or a particular epitope within an antigen with greater selectivity and reproducibility. By means of example and not limitation, monoclonal antibodies may be made by the hybridoma method first described by Kohler et al. 1975 (Nature 256: 495), or may be made by recombinant DNA methods (e.g., as in U.S. Pat. No. 4,816,567). Monoclonal antibodies may also be isolated from phage antibody libraries using techniques as described by Clackson et al. 1991 (Nature 352: 624-628) and Marks et al. 1991 (J Mol Biol 222: 581-597), for example.

Antibody binding agents may be antibody fragments. “Antibody fragments” comprise a portion of an intact antibody, comprising the antigen-binding or variable region thereof. Examples of antibody fragments include Fab, Fab′, F(ab′)2, Fv and scFv fragments, single domain (sd) Fv, such as VH domains, VL domains and VHH domains; diabodies; linear antibodies; single-chain antibody molecules, in particular heavy-chain antibodies; and multivalent and/or multispecific antibodies formed from antibody fragment(s), e.g., dibodies, tribodies, and multibodies. The above designations Fab, Fab′, F(ab′)2, Fv, scFv etc. are intended to have their art-established meaning.

The term antibody includes antibodies originating from or comprising one or more portions derived from any animal species, preferably vertebrate species, including, e.g., birds and mammals. Without limitation, the antibodies may be chicken, turkey, goose, duck, guinea fowl, quail or pheasant. Also without limitation, the antibodies may be human, murine (e.g., mouse, rat, etc.), donkey, rabbit, goat, sheep, guinea pig, camel (e.g., Camelus bactrianus and Camelus dromaderius), llama (e.g., Lama paccos, Lama glama or Lama vicugna) or horse.

A skilled person will understand that an antibody can include one or more amino acid deletions, additions and/or substitutions (e.g., conservative substitutions), insofar such alterations preserve its binding of the respective antigen. An antibody may also include one or more native or artificial modifications of its constituent amino acid residues (e.g., glycosylation, etc.).

Methods of producing polyclonal and monoclonal antibodies as well as fragments thereof are well known in the art, as are methods to produce recombinant antibodies or fragments thereof (see for example, Harlow and Lane, “Antibodies: A Laboratory Manual”, Cold Spring Harbour Laboratory, New York, 1988; Harlow and Lane, “Using Antibodies: A Laboratory Manual”, Cold Spring Harbour Laboratory, New York, 1999, ISBN 0879695447; “Monoclonal Antibodies: A Manual of Techniques”, by Zola, ed., CRC Press 1987, ISBN 0849364760; “Monoclonal Antibodies: A Practical Approach”, by Dean & Shepherd, eds., Oxford University Press 2000, ISBN 0199637229; Methods in Molecular Biology, vol. 248: “Antibody Engineering: Methods and Protocols”, Lo, ed., Humana Press 2004, ISBN 1588290921).

In certain embodiments, the agent may be a Nanobody®. The terms “Nanobody®” and “Nanobodies®” are trademarks of Ablynx NV (Belgium). The term “Nanobody” is well-known in the art and as used herein in its broadest sense encompasses an immunological binding agent obtained (1) by isolating the V_{HH} domain of a heavy-chain antibody, preferably a heavy-chain antibody derived from camelids; (2) by expression of a nucleotide sequence encoding a V_{HH} domain; (3) by “humanization” of a naturally occurring V_{HH} domain or by expression of a nucleic acid encoding a such humanized V_{HH} domain; (4) by “camelization” of a V_{H} domain from any animal species, and in particular from a mammalian species, such as from a human being, or by expression of a nucleic acid encoding such a camelized V_{H} domain; (5) by “camelization” of a “domain antibody” or “dAb” as described in the art, or by expression of a nucleic acid encoding such a camelized dAb; (6) by using synthetic or semi-synthetic techniques for preparing proteins, polypeptides or other amino acid sequences known per se; (7) by preparing a nucleic acid encoding a Nanobody using techniques for nucleic acid synthesis known per se, followed by expression of the nucleic acid thus obtained; and/or (8) by any combination of one or more of the foregoing. “Camelids” as used herein comprise old world camelids (Camelus bactrianus and Camelus dromaderius) and new world camelids (for example Lama paccos, Lama glama and Lama vicugna).

Although in general, antibody-like scaffolds have proven to work well as specific binders, it has become apparent that it is not compulsory to stick strictly to the paradigm of a rigid scaffold that displays CDR-like loops. In addition to antibodies, many other natural proteins mediate specific high-affinity interactions between domains. Alternatives to immunoglobulins have provided attractive starting points for the design of novel binding (recognition) molecules. The term scaffold, as used herein, refers to a protein framework that can carry altered amino acids or sequence insertions that confer binding to specific target proteins. Engineering scaffolds and designing libraries are mutually interdependent processes. In order to obtain specific binders, a combinatorial library of the scaffold has to be generated. This is usually done at the DNA level by randomizing the codons at appropriate amino acid positions, by using either degenerate codons or trinucleotides. A wide range of different non-immunoglobulin scaffolds with widely diverse origins and characteristics are currently used for combinatorial library display. Some of them are comparable in size to a scFv of an antibody (about 30 kDa), while the majority of them are much smaller. Modular scaffolds based on repeat proteins vary in size depending on the number of repetitive units. A non-limiting list of examples comprise binders based on the human 10th fibronectin type III domain, binders based on lipocalins, binders based on SH3 domains, binders based on members of the knottin family, binders based on CTLA-4, T-cell receptors, neocarzinostatin, carbohydrate binding module 4-2, tendamistat, kunitz domain inhibitors, PDZ domains, Src homology domain (SH2), scorpion toxins, insect defensin A, plant homeodomain finger proteins, bacterial enzyme TEM-1 beta-lactamase, Ig-binding domain of Staphylococcus aureus protein A, E. coli colicin E7 immunity protein, E. coli cytochrome b562, ankyrin repeat domains. Hence, the term “antibody-like protein scaffolds” or “engineered protein scaffolds” broadly encompasses proteinaceous non-immunoglobulin specific-binding agents, typically obtained by combinatorial engineering (such as site-directed random mutagenesis in combination with phage display or other molecular selection techniques). Usually, such scaffolds are derived from robust and small soluble monomeric proteins (such as Kunitz inhibitors or lipocalins) or from a stably folded extra-membrane domain of a cell surface receptor (such as protein A, fibronectin or the ankyrin repeat). Such scaffolds have been extensively reviewed in Binz et al., Gebauer and Skerra, Gill and Damle, Skerra 2000, and Skerra 2007, and include without limitation affibodies, based on the Z-domain of staphylococcal protein A, a three-helix bundle of 58 residues providing an interface on two of its alpha-helices (Nygren); engineered Kunitz domains based on a small (ca. 58 residues) and robust, disulphide-crosslinked serine protease inhibitor, typically of human origin (e.g. LACI-D1), which can be engineered for different protease specificities (Nixon and Wood); monobodies or adnectins based on the 10th extracellular domain of human fibronectin III (10Fn3), which adopts an Ig-like beta-sandwich fold (94 residues) with 2-3 exposed loops, but lacks the central disulphide bridge (Koide and Koide); anticalins derived from the lipocalins, a diverse family of eight-stranded beta-barrel proteins (ca. 180 residues) that naturally form binding sites for small ligands by means of four structurally variable loops at the open end, which are abundant in humans, insects, and many other organisms (Skerra 2008); DARPins, designed ankyrin repeat domains (166 residues), which provide a rigid interface arising from typically three repeated beta-turns (Stumpp et al.); avimers (multimerized LDLR-A module) (Silverman et al.); and cysteine-rich knottin peptides (Kolmar). Also included as binding domains are compounds with a specificity for a given target protein, cyclic and linear peptide binders, peptide aptamers, multivalent avimer proteins or small modular immunopharmaceutical drugs, ligands with a specificity for a receptor or a co-receptor, protein binding partners identified in a two-hybrid analysis, binding domains based on the specificity of the biotin-avidin high affinity interaction, binding domains based on the specificity of cyclophilin-FK506 binding proteins. Also included are lectins with an affinity for a specific carbohydrate structure.

By means of an example, mutations of proto-oncogenes are often found in cancers, and monoclonal antibodies fused to the present molecules may be configured to specifically bind a protein expressed by tumor cells in a subject, such as a tumor antigen, preferably a surface tumor antigen.

The term “tumor antigen” refers to an antigen that is uniquely or differentially expressed by a tumor cell, whether intracellular or on the tumor cell surface (preferably on the tumor cell surface), compared to a normal or non-neoplastic cell. By means of example, a tumor antigen may be present in or on a tumor cell and not typically in or on normal cells or non-neoplastic cells (e.g., only expressed by a restricted number of normal tissues, such as testis and/or placenta), or a tumor antigen may be present in or on a tumor cell in greater amounts than in or on normal or non-neoplastic cells, or a tumor antigen may be present in or on tumor cells in a different form than that found in or on normal or non-neoplastic cells. The term thus includes tumor-specific antigens (TSA), including tumor-specific membrane antigens, tumor-associated antigens (TAA), including tumor-associated membrane antigens, embryonic antigens on tumors, growth factor receptors, growth factor ligands, etc. The term further includes cancer/testis (CT) antigens. Examples of tumor antigens include, without limitation, β-human chorionic gonadotropin (βHCG), glycoprotein 100 (gp100/Pme117), carcinoembryonic antigen (CEA), tyrosinase, tyrosinase-related protein 1 (gp75/TRP1), tyrosinase-related protein 2 (TRP-2), NY-BR-1, NY-CO-58, NY-ESO-1, MN/gp250, idiotypes, telomerase, synovial sarcoma X breakpoint 2 (SSX2), mucin 1 (MUC-1), antigens of the melanoma-associated antigen (MAGE) family, high molecular weight-melanoma associated antigen (HMW-MAA), melanoma antigen recognized by T cells 1 (MARTI), Wilms' tumor gene 1 (WT1), HER2/neu, mesothelin (MSLN), alphafetoprotein (AFP), cancer antigen 125 (CA-125), and abnormal forms of ras or p53. Further targets in neoplastic diseases include without limitation CD37 (chronic lymphocytic leukemia), CD123 (acute myeloid leukemia), CD30 (Hodgkin/large cell lymphoma), MET (NSCLC, gastroesophageal cancer), IL-6 (NSCLC), and GITR (malignant melanoma).

In those instances where other moieties are fused to the molecules, it is envisaged in particular embodiments that these moieties can be removed from the molecule. Typically, this will be done through incorporating a specific protease cleavage site or an equivalent approach. This is particularly the case where the moiety is a large protein: in such cases, the moiety may be cleaved off prior to using the molecule in any of the methods described herein (e.g. during purification of the molecules).

Note however that targeting moieties are not necessary, as the molecules themselves are able to find their target through specific sequence recognition. This may also allow, in alternative embodiments, to employ the molecules can as targeting moiety and be further fused to other moieties such as drugs, toxins or small molecules. By targeting the molecules to the mutant or variant protein, these compounds can be targeted to the specific cell type/compartment. Thus, for instance, toxins can selectively be delivered to cancer cells expressing a mutated proto-oncogene.

As the present invention makes use of the ‘interferor’ technology as generally described in WO 2007/071789A1 and WO2012/123419A1, and adopts this technology to the novel situations in which a mutant or variant form of a protein contains an APR different from an APR in the unmodified protein or a de novo APR, it shall be appreciated that the teachings of WO 2007/071789A1 and WO2012/123419A1 concerning the manners in which such ‘interferor’ molecules can be produced, isolated, purified, stored and formulated can be applied in the context of the present invention and need not be elaborated in great detail herein.

As mentioned, in particular embodiments, the operative part of the molecule may comprise, consist essentially of or consist of a peptide, preferably the operative part of the molecule may be a peptide. Moreover, in many embodiments, for example, where the operative part of the molecule is not connected or fused to other auxiliary moieties or where such additional moiety or moieties are themselves peptides, the entire molecule may be a peptide. Accordingly, standards tools and methods of chemical peptide synthesis, or of recombinant peptide or polypeptide production can be applied to the preparation of the present molecules. Recombinant protein production can also be applied to preparing molecules in which additional moiety or moieties which are themselves proteinaceous are included in the molecules and fused to the operative part of the molecule by peptide bonds.

Given that such techniques have become generally routine, in the interest of brevity, recombinant production of the present molecules may employ an expression cassette or expression vector comprising a nucleic acid encoding the molecule as taught herein and a promoter operably linked to the nucleic acid, wherein the expression cassette or expression vector is configured to effect expression of the molecule in a suitable host cell, such as a bacterial cell, a fungal cell, including yeast cells, an animal cell, or a mammalian cell, including human cells and non-human mammalian cells. Vectors may include plasmids, phagemids, bacteriophages, bacteriophage-derived vectors, PAC, BAC, linear nucleic acids, e.g., linear DNA, or viral vectors, etc. Expression vectors can be autonomous or integrative. Expression vectors can contain selection marker(s), e.g., URA3, TRP1, to permit detection and/or selection of the transformed cells. An operable linkage is a linkage in which regulatory sequences and sequences sought to be expressed are connected in such a way as to permit said expression. The promotor may be a constitutive or inducible (conditional) promoter, e.g., a chemically regulated or physically regulated inducible promoter. Non-limiting examples of promoters include T7, U6, H1, retroviral Rous sarcoma virus (RSV) LTR promoter, the cytomegalovirus (CMV) promoter, the metallothionein promoter, the adenovirus late promoter, the SV40 promoter, the dihydrofolate reductase promoter, the β-actin promoter, the phosphoglycerol kinase (PGK) promoter, and the EF1α promoter. Transcription terminators and optionally transcription enhancers may be included. A recombinant nucleic acid can be introduced into a host cell using a variety of methods such as direct injection, protoplasts fusion, calcium chloride, rubidium chloride, lithium chloride, calcium phosphate, DEAE dextran, cationic lipids or liposomes, biolistic particle bombardment (“gene gun” method), infection with viral vectors (e.g., derived from lentivirus, adeno-associated virus (AAV), adenovirus, retrovirus or antiviruses), electroporation, etc. Expression systems (host cells) that can be used for small or large scale production of peptides or polypeptides include, without limitation, microorganisms such as bacteria (e.g., Escherichia coli, Yersinia enterocolitica, Brucella sp., Salmonella typhimurium, Serratia marcescens, or Bacillus subtilis), fungal cells (e.g., Yarrowia lipolytica, Arxula adeninivorans, methylotrophic yeast (e.g., methylotrophic yeast of the genus Candida, Hansenula, Oogataea, Pichia or Torulopsis, e.g., Pichia pastoris, Hansenula polymorpha, Ogataea minuta, or Pichia methanolica), or filamentous fungi of the genus Aspergillus, Trichoderma, Neurospora, Fusarium, or Chrysosporium, e.g., Aspergillus niger, Trichoderma reesei, or yeast of the genus Saccharomyces or Schizosaccharomyces, e.g., Saccharomyces cerevisiae, or Schizosaccharomyces pombe), insect cell systems (e.g., cells derived from Drosophila melanogaster, such as Schneider 2 cells, cell lines derived from the army worm Spodoptera frugiperda, such as Sf9 and Sf21 cells, or cells derived from the cabbage looper Trichoplusia ni, such as High Five cells), plant cell systems infected with recombinant virus expression vectors (e.g., tobacco mosaic virus) or transformed with recombinant plasmid expression vectors (e.g., Ti plasmid). Mammalian expression systems include human and non-human mammalian cells, such as rodent cells, primate cells, or human cells. Mammalian cells, such as human or non-human mammalian cells, may include primary cells, secondary, tertiary etc. cells, or may include immortalised cell lines, including clonal cell lines. Preferred animal cells can be readily maintained and transformed in tissue culture. Non-limiting example of human cells include the human HeLa (cervical cancer) cell line. Other human cell lines common in tissue culture practice include inter alia human embryonic kidney 293 cells (HEK cells), DU145 (prostate cancer), Lncap (prostate cancer), MCF-7 (breast cancer), MDA-MB-438 (breast cancer), PC3 (prostate cancer), T47D (breast cancer), THP-1 (acute myeloid leukemia), U87 (glioblastoma), SHSY5Y (neuroblastoma), or Saos-2 cells (bone cancer). A non-limiting example of primate cells are Vero (African green monkey Chlorocebus kidney epithelial cell line) cells, and COS cells. Non-limiting examples of rodent cells are rat GH3 (pituitary tumor), CHO (Chinese hamster ovary), PC12 (pheochromocytoma) cell lines, or mouse MC3T3 (embryonic calvarium) cell line.

Any molecules, such as proteins, polypeptides or peptides as prepared herein can be suitably purified. The term “purified” with reference to molecules, peptides, polypeptides or proteins does not require absolute purity. Instead, it denotes that such molecules, peptides, polypeptides or proteins are in a discrete environment in which their abundance (conveniently expressed in terms of mass or weight or concentration) relative to other components is greater than in the starting composition or sample, e.g., in the production sample, such as in a lysate or supernatant of a recombinant host cells producing the molecule, peptide, polypeptide or protein. A discrete environment denotes a single medium, such as for example a single solution, gel, precipitate, lyophilisate, etc. Purified molecules, proteins, polypeptides or peptides may be obtained by known methods including, for example, chemical synthesis, chromatography, preparative electrophoresis, centrifugation, precipitation, affinity purification, etc. Purified molecules, peptides, polypeptides or proteins may preferably constitute by weight≥10%, more preferably ≥50%, such as ≥60%, yet more preferably ≥70%, such as ≥80%, and still more preferably ≥90%, such as ≥95%, ≥96%, ≥97%, ≥98%, ≥99% or even 100%, of the non-solvent content of the discrete environment. For example, purified peptides, polypeptides or proteins may preferably constitute by weight≥10%, more preferably ≥50%, such as ≥60%, yet more preferably ≥70%, such as ≥80%, and still more preferably ≥90%, such as ≥95%, ≥96%, ≥97%, ≥98%, ≥99% or even 100%, of the protein content of the discrete environment. Protein content may be determined, e.g., by the Lowry method (Lowry et al. 1951. J Biol Chem 193: 265), optionally as described by Hartree 1972 (Anal Biochem 48: 422-427). Purity of peptides, polypeptides, or proteins may be determined by HPLC, or SDS-PAGE under reducing or non-reducing conditions using Coomassie blue or, preferably, silver stain.

Any molecules, such as proteins, polypeptides or peptides as prepared herein can be suitably kept in solution in deionised water, or in deionised water with DMSO, e.g., 50% v/v DMSO in deionised water, or in an aqueous solution, or in a suitable buffer, such as in a buffer having physiological pH, or at pH between 5 and 9, more particular pH between 6 and 8, such as in neutral buffered saline, phosphate buffered saline, Tris-HCl, acetate or phosphate buffers, or in a strong chaotropic agent such as 6M urea, at concentrations of the molecules convenient for downstream use, such as without limitation between about 1 mM and about 500 mM, or between about 1 mM and about 250 mM, or between about 1 mM and about 100 mM, or between about 5 mM and about 50 mM, or between about 5 mM and about 20 mM. Alternatively, any molecules, such as proteins, polypeptides or peptides as prepared herein may be lyophilised as is generally known in the art. Storage may typically be at or below room temperature (at or below 25° C.), in certain embodiments at temperatures above 0° C. (non-cryogenic storage), such as at a temperature above 0° C. and not exceeding 25° C., or in certain embodiments cryopreservation may be preferred, at temperatures of 0° C. or lower, typically −5° C. or lower, more typically −10° C. or lower, such as −20° C. or lower, −25° C. or lower, −30° C. or lower, or even at −70° C. or lower or −80° C. or lower, or in liquid nitrogen.

Recombinant nucleic acid technology may allow not only for heterologous expression and isolation of pept-ins which are of polypeptide nature and are encoded by the nucleic acids, but may even allow to administer such pept-ins as transgenes, i.e., to administer nucleic acids (such as, for example, DNA-based or RNA-based cassettes, vectors or constructs) encoding the respective pept-ins and capable of effecting the expression of the respective pept-ins when introduced into a cell. For example, in a DNA construct a pept-in coding sequence may be operably linked to regulatory sequence(s) configured to drive the transcription and translation of the pept-in from the DNA construct, such as a promoter and a transcription terminator. In an RNA or mRNA construct a pept-in coding sequence may be included such that it can be translated by the cellular protein translation machinery. In aforementioned constructs a pept-in coding sequence will be typically preceded by an in-frame translation initiation codon and followed by a translation termination codon, to facilitate proper translation. Accordingly, wherever administration of/introduction of/therapy with pept-ins as taught herein is envisaged in this specification, the administration of/introduction of nucleic acids encoding those pept-ins to cells or organisms is encompassed by the disclosure. Such administration/introduction/therapy may commonly be referred to as gene therapy or gene transformation or genetic modification. Thus all methods and uses involving the molecules of the application thus also encompass methods and uses where the molecules are provided as the nucleic acid sequence encoding them, and the molecules are expressed from the nucleic acid sequence.

Hence, also provided herein is a nucleic acid encoding any pept-in molecule as disclosed herein, where such pept-in molecule is of polypeptide nature. It is particularly envisaged that the nucleic acid sequences encode the molecules with all the features and variations described herein, mutatis mutandis. Thus, the encoded polypeptide is in essence as described herein, that is to say, the variations mentioned for the pept-in molecules that are compatible with this aspect are also envisaged as variations for the polypeptides encoded by the nucleic acid sequences.

In certain embodiments, the nucleic acid sequence is an artificial gene. Since the nucleic acid aspect is most particularly suitable in applications making use of transgenic expression, particularly envisaged embodiments may be those where the nucleic acid sequence (or the artificial gene) is fused to another moiety, particularly a moiety that increases solubility and/or stability of the gene product.

Also provided in this aspect are recombinant vectors comprising such a nucleic acid sequence encoding a molecule as herein described. These recombinant vectors are ideally suited as a vehicle to carry the nucleic acid sequence of interest inside a cell where the protein to be downregulated is expressed, and drive expression of the nucleic acid in said cell. The recombinant vector may persist as a separate entity in the cell (e.g., as a plasmid), or may be integrated into the genome of the cell. Recombinant vectors include among others plasmid vectors, binary vectors, cloning vectors, expression vectors, shuttle vectors and viral vectors. Thus, also encompassed herein are methods and uses where the molecules are provided as recombinant vectors with a nucleic acid sequence encoding the molecules, and the molecules are expressed from the nucleic acid sequence provided in the recombinant vector. Accordingly, cells are provided herein comprising a nucleic acid sequence encoding a molecule as herein described, or comprising a recombinant vector that contains a nucleic acid sequence encoding such pept-in molecule. The cell may be a prokaryotic or eukaryotic cell. In the latter case, it may be a yeast, algae, plant or animal cell (e.g. insect, mammal or human cell). Thus, also encompassed herein are methods and uses where the molecules are provided as cells with a nucleic acid sequence encoding the molecules, and the molecules are expressed from the nucleic acid sequence provided in the cells. This can, e.g., be the case in stem cell therapy.

Such transgenic approaches are not limited to medical applications. According to particular embodiments, the provision of pept-in molecules encoded in nucleic acid instead of directly as polypeptides may be particularly suited for use in plants. Accordingly, plants, or plant cells, or plant seeds, are provided herein that contain a nucleic acid sequence, artificial gene or a recombinant vector as described herein. Also plant protoplasts containing such sequences are envisaged herein.

As discussed above, the present proteins and their mutant or variant forms may be of any organism, structure or function—as long as there exists a distinction in the APR profile of the protein vs. its mutant or variant form, this can be exploited to design APR-targeting molecules to specifically downregulate the latter form. In other words, the invention is broadly applicable to any situation in which a mutant or variant form of a protein may be an interesting object for downregulation.

In certain embodiments, particularly in medical applications in humans or in veterinary applications in animals, such in vertebrates such as preferably non-human mammals, the mutant or variant form of the protein may be causative of or associated with a disease. The reference to a disease caused by or associated with the mutant or variant form of the protein intends to broadly encompass any disease in which the mutation or variation plays at least some part in the disease, and therefore in which downregulation of the mutant or variant form of the protein could be of therapeutic benefit. For example, the mutation or variation may be solely, or jointly with other factors such as other mutations, responsible for or contribute to the aetiology of the disease, and/or the mutation or variation may be solely, or jointly with other factors such as other mutations, responsible for or contribute to the persistence, progression, worsening, resistance to other treatments or reappearance of the disease.

In certain preferred embodiments, the disease may be a neoplastic disease, particularly cancer.

The term “neoplastic disease” generally refers to any disease or disorder characterised by neoplastic cell growth and proliferation, whether benign (not invading surrounding normal tissues, not forming metastases), pre-malignant (pre-cancerous), or malignant (invading adjacent tissues and capable of producing metastases). The term neoplastic disease generally includes all transformed cells and tissues and all cancerous cells and tissues. Neoplastic diseases or disorders include, but are not limited to abnormal cell growth, benign tumors, premalignant or precancerous lesions, malignant tumors, and cancer. Examples of neoplastic diseases or disorders are benign, pre-malignant, or malignant neoplasms located in any tissue or organ, such as in the prostate, colon, abdomen, bone, breast, digestive system, liver, pancreas, peritoneum, endocrine glands (adrenal, parathyroid, pituitary, testicles, ovary, thymus, thyroid), eye, head and neck, nervous (central and peripheral), lymphatic system, pelvic, skin, soft tissue, spleen, thoracic, or urogenital tract.

As used herein, the terms “tumor” or “tumor tissue” refer to an abnormal mass of tissue that results from excessive cell division. A tumor or tumor tissue comprises tumor cells which are neoplastic cells with abnormal growth properties and no useful bodily function. Tumors, tumor tissue and tumor cells may be benign, pre-malignant or malignant, or may represent a lesion without any cancerous potential. A tumor or tumor tissue may also comprise tumor-associated non-tumor cells, e.g., vascular cells which form blood vessels to supply the tumor or tumor tissue. Non-tumor cells may be induced to replicate and develop by tumor cells, for example, the induction of angiogenesis in a tumor or tumor tissue.

As used herein, the term “cancer” refers to a malignant neoplasm characterised by deregulated or unregulated cell growth. The term “cancer” includes primary malignant cells or tumors (e.g., those whose cells have not migrated to sites in the subject's body other than the site of the original malignancy or tumor) and secondary malignant cells or tumors (e.g., those arising from metastasis, the migration of malignant cells or tumor cells to secondary sites that are different from the site of the original tumor). The term “metastatic” or “metastasis” generally refers to the spread of a cancer from one organ or tissue to another non-adjacent organ or tissue. The occurrence of the neoplastic disease in the other non-adjacent organ or tissue is referred to as metastasis.

Examples of cancer include but are not limited to carcinoma, lymphoma, blastoma, sarcoma, and leukemia or lymphoid malignancies. More particular examples of such cancers include without limitation: squamous cell cancer (e.g., epithelial squamous cell cancer), lung cancer including small-cell lung cancer, non-small cell lung cancer, adenocarcinoma of the lung, squamous carcinoma of the lung and large cell carcinoma of the lung, cancer of the peritoneum, hepatocellular cancer, gastric or stomach cancer including gastrointestinal cancer, pancreatic cancer, glioma, glioblastoma, cervical cancer, ovarian cancer, liver cancer, bladder cancer, hepatoma, breast cancer, colon cancer, rectal cancer, colorectal cancer, endometrial cancer or uterine carcinoma, salivary gland carcinoma, kidney or renal cancer, prostate cancer, vulvar cancer, thyroid cancer, hepatic carcinoma, anal carcinoma, penile carcinoma, as well as CNS cancer, melanoma, head and neck cancer, bone cancer, bone marrow cancer, duodenum cancer, esophageal cancer, thyroid cancer, or hematological cancer.

Other non-limiting examples of cancers or malignancies include, but are not limited to: Acute Childhood Lymphoblastic Leukemia, Acute Lymphoblastic Leukemia, Acute Lymphocytic Leukemia, Acute Myeloid Leukemia, Adrenocortical Carcinoma, Adult (Primary) Hepatocellular Cancer, Adult (Primary) Liver Cancer, Adult Acute Lymphocytic Leukemia, Adult Acute Myeloid Leukemia, Adult Hodgkin's Disease, Adult Hodgkin's Lymphoma, Adult Lymphocytic Leukemia, Adult Non-Hodgkin's Lymphoma, Adult Primary Liver Cancer, Adult Soft Tissue Sarcoma, AIDS-Related Lymphoma, AIDS-Related Malignancies, Anal Cancer, Astrocytoma, Bile Duct Cancer, Bladder Cancer, Bone Cancer, Brain Stem Glioma, Brain Tumors, Breast Cancer, Cancer of the Renal Pelvis and Urethra, Central Nervous System (Primary) Lymphoma, Central Nervous System Lymphoma, Cerebellar Astrocytoma, Cerebral Astrocytoma, Cervical Cancer, Childhood (Primary) Hepatocellular Cancer, Childhood (Primary) Liver Cancer, Childhood Acute Lymphoblastic Leukemia, Childhood Acute Myeloid Leukemia, Childhood Brain Stem Glioma, Glioblastoma, Childhood Cerebellar Astrocytoma, Childhood Cerebral Astrocytoma, Childhood Extracranial Germ Cell Tumors, Childhood Hodgkin's Disease, Childhood Hodgkin's Lymphoma, Childhood Hypothalamic and Visual Pathway Glioma, Childhood Lymphoblastic Leukemia, Childhood Medulloblastoma, Childhood Non-Hodgkin's Lymphoma, Childhood Pineal and Supratentorial Primitive Neuroectodermal Tumors, Childhood Primary Liver Cancer, Childhood Rhabdomyosarcoma, Childhood Soft Tissue Sarcoma, Childhood Visual Pathway and Hypothalamic Glioma, Chronic Lymphocytic Leukemia, Chronic Myelogenous Leukemia, Colon Cancer, Cutaneous T-Cell Lymphoma, Endocrine Pancreas Islet Cell Carcinoma, Endometrial Cancer, Ependymoma, Epithelial Cancer, Esophageal Cancer, Ewing's Sarcoma and Related Tumors, Exocrine Pancreatic Cancer, Extracranial Germ Cell Tumor, Extragonadal Germ Cell Tumor, Extrahepatic Bile Duct Cancer, Eye Cancer, Female Breast Cancer, Gallbladder Cancer, Gastric Cancer, Gastrointestinal Carcinoid Tumor, Gastrointestinal Tumors, Germ Cell Tumors, Gestational Trophoblastic Tumor, Hairy Cell Leukemia, Head and Neck Cancer, Hepatocellular Cancer, Hodgkin's Disease, Hodgkin's Lymphoma, Hypergammaglobulinemia, Hypopharyngeal Cancer, Intestinal Cancers, Intraocular Melanoma, Islet Cell Carcinoma, Islet Cell Pancreatic Cancer, Kaposi's Sarcoma, Kidney Cancer, Laryngeal Cancer, Lip and Oral Cavity Cancer, Liver Cancer, Lung Cancer, Lymphoproliferative Disorders, Macroglobulinemia, Male Breast Cancer, Malignant Mesothelioma, Malignant Thymoma, Medulloblastoma, Melanoma, Mesothelioma, Metastatic Occult Primary Squamous Neck Cancer, Metastatic Primary Squamous Neck Cancer, Metastatic Squamous Neck Cancer, Multiple Myeloma, Multiple Myeloma/Plasma Cell Neoplasm, Myelodysplastic Syndrome, Myelogenous Leukemia, Myeloid Leukemia, Myeloproliferative Disorders, Nasal Cavity and Paranasal Sinus Cancer, Nasopharyngeal Cancer, Neuroblastoma, Non-Hodgkin's Lymphoma During Pregnancy, Non-melanoma Skin Cancer, Non-Small Cell Lung Cancer, Occult Primary Metastatic Squamous Neck Cancer, Oropharyngeal Cancer, Osteo-/Malignant Fibrous Sarcoma, Osteosarcoma/Malignant Fibrous Histiocytoma, Osteosarcoma/Malignant Fibrous Histiocytoma of Bone, Ovarian Epithelial Cancer, Ovarian Germ Cell Tumour, Ovarian Low Malignant Potential Tumor, Pancreatic Cancer, Paraproteinemias, Purpura, Parathyroid Cancer, Penile Cancer, Pheochromocytoma, Pituitary Tumor, Plasma Cell Neoplasm/Multiple Myeloma, Primary Central Nervous System Lymphoma, Primary Liver Cancer, Prostate Cancer, Rectal Cancer, Renal Cell Cancer, Renal Pelvis and Urethra Cancer, Retinoblastoma, Rhabdomyosarcoma, Salivary Gland Cancer, Sarcoidosis Sarcomas, Sezary Syndrome, Skin Cancer, Small Cell Lung Cancer, Small Intestine Cancer, Soft Tissue Sarcoma, Squamous Neck Cancer, Stomach Cancer, Supratentorial Primitive Neuroectodermal and Pineal Tumors, T-Cell Lymphoma, Testicular Cancer, Thymoma, Thyroid Cancer, Transitional Cell Cancer of the Renal Pelvis and Urethra, Transitional Renal Pelvis and Urethra Cancer, Trophoblastic Tumours, Urethra and Renal Pelvis Cell Cancer, Urethral Cancer, Uterine Cancer, Uterine Sarcoma, Vaginal Cancer, Visual Pathway and Hypothalamic Glioma, Vulvar Cancer, Waldenstrom's Macroglobulinemia, or Wilms' Tumour.

In certain embodiments, the protein may be a proto-oncogene and the mutant or variant form of the protein may be an oncogene, which causes or contributes to the neoplastic transformation of a cell. This also encompasses the situation in which the protein is a tumor suppressor gene, and the mutant or variant form of the protein promotes the neoplastic transformation of a cell, especially by a gain-of-function or dominant negative mechanism. The mutation or variation may be germline or somatic. Such proto-oncogenes or tumor suppressor genes, as well as tumorigenic mutations therein, are well-known and comprehensively annotated in the databases mentioned above. Examples of known proto-oncogenes include without limitation HER-2/neu, EGFR, VEGF, PDGFR, BCR/ABL, C-KIT, KRAS, HRAS, NRAS, Cyclin D1, Cyclin E, MYC, beta-Catenin, B-RAF, MITF, GNAS, MP2K2, IDHP, ITK, ERBB2, etc. which can be targetable insofar an altered APR as explained throughout this specification is produced by the mutation. Examples of known proto-oncogenes include without limitation p53, CDKN2A/CDKN2B, PTEN, pRb, BCL2, INK4a, NM23, SWI/SNF, pVHL, PARP, CIP2A, APC, CD95, ST5, YPEL3, ST7, ST14, p16, BRCA1/BRCA2, and APC. In certain cases, mutations occurring in tumor suppressor genes may increase the aggregation propensity of APRs, which drives the aggregation and thus downregulation of the mutant tumor suppressor protein in cancer cells (and potentially a dominant negative effect if the wild-type tumor suppressor protein is also sequestered into such aggregates). The present molecules, which aim to induce aggregation of target mutant or variant proteins, may thus typically not be applied in such situations, since inducing further aggregation of the already aggregating mutant tumor suppressor protein would not normally be expected to have a beneficial effect on the disease.

Hence, the molecules as taught herein may be useful for therapy. An aspect thus provides any molecule as taught herein for use in medicine, or in other words, any molecule as taught herein for use in therapy. As discussed below, the molecules as taught herein can be formulated into pharmaceutical compositions. Therefore, any reference to the use of the molecules in therapy (or any variation of such language) also subsumes the use of pharmaceutical compositions comprising the molecules in therapy.

In particular, the molecules are intended for therapy of afflictions in which the mutant or variant form of the protein plays an important role. Accordingly, also provided is any molecule as taught herein for use in a method of treating a disease caused by or associated with the mutant or variant form of the protein. Further provided is a method for treating a subject in need thereof, in particularly a subject having a disease caused by or associated with the mutant or variant form of the protein, the method comprising administering to the subject a therapeutically effective amount of the respective molecule as taught herein. Further provided is use of the respective molecule as taught herein for the manufacture of a medicament for the treatment of a disease caused by or associated with the mutant or variant form of the protein. Further provided is use of the respective molecule as taught herein for the treatment of a disease caused by or associated with the mutant or variant form of the protein.

Reference to “therapy” or “treatment” broadly encompasses both curative and preventative treatments, and the terms may particularly refer to the alleviation or measurable lessening of one or more symptoms or measurable markers of a pathological condition such as a disease or disorder. The terms encompass primary treatments as well as neo-adjuvant treatments, adjuvant treatments and adjunctive therapies. Measurable lessening includes any statistically significant decline in a measurable marker or symptom. Generally, the terms encompass both curative treatments and treatments directed to reduce symptoms and/or slow progression of the disease. The terms encompass both the therapeutic treatment of an already developed pathological condition, as well as prophylactic or preventative measures, wherein the aim is to prevent or lessen the chances of incidence of a pathological condition. In certain embodiments, the terms may relate to therapeutic treatments. In certain other embodiments, the terms may relate to preventative treatments. Treatment of a chronic pathological condition during the period of remission may also be deemed to constitute a therapeutic treatment. The term may encompass ex vivo or in vivo treatments as appropriate in the context of the present invention.

The terms “subject”, “individual” or “patient” are used interchangeably throughout this specification, and typically and preferably denote humans, but may also encompass reference to non-human animals, preferably warm-blooded animals, even more preferably non-human mammals. Particularly preferred are human subjects including both genders and all age categories thereof. In other embodiments, the subject is an experimental animal or animal substitute as a disease model. The term does not denote a particular age or sex. Thus, adult and newborn subjects, as well as fetuses, whether male or female, are intended to be covered. The term subject is further intended to include transgenic non-human species.

The term “subject in need of treatment” or similar as used herein refers to subjects diagnosed with or having a disease as recited herein and/or those in whom said disease is to be prevented.

The term “therapeutically effective amount” generally denotes an amount sufficient to elicit the pharmacological effect or medicinal response in a subject that is being sought by a medical practitioner such as a medical doctor, clinician, surgeon, veterinarian, or researcher, which may include inter alia alleviation of the symptoms of the disease being treated, in either a single or multiple doses. Appropriate therapeutically effective doses of the present molecules may be determined by a qualified physician with due regard to the nature and severity of the disease, and the age and condition of the patient. The effective amount of the molecules described herein to be administered can depend on many different factors and can be determined by one of ordinary skill in the art through routine experimentation. Several non-limiting factors that might be considered include biological activity of the active ingredient, nature of the active ingredient, characteristics of the subject to be treated, etc. The term “to administer” generally means to dispense or to apply, and typically includes both in vivo administration and ex vivo administration to a tissue, preferably in vivo administration. Generally, compositions may be administered systemically or locally.

As stated earlier, the mutant or variant protein may be causative of or associated with a neoplastic disease, e.g., an oncogene or a mutated tumor suppressor gene. Accordingly, also provided is the respective molecule as taught herein for use in a method of treating a neoplastic disease, particularly cancer, caused by or associated with the mutant or variant form of the protein. Further provided is a method for treating a subject in need thereof, in particular a subject having a neoplastic disease, particularly cancer, caused by or associated with the mutant or variant form of the protein, the method comprising administering to the subject a therapeutically effective amount of any molecule as taught herein. Further provided is use of any molecule as taught herein for the manufacture of a medicament for the treatment of a neoplastic disease, particularly cancer, caused by or associated with the mutant or variant form of the protein. Further provided is use of any molecule as taught herein for the treatment of a neoplastic disease, particularly cancer, caused by or associated with the mutant or variant form of the protein.

In certain embodiments, any molecule as taught herein may be administered as the sole pharmaceutical agent (active pharmaceutical ingredient) or in combination with one or more other pharmaceutical agents where the combination causes no unacceptable adverse effects. By means of an example, two or more molecules as taught herein may be co-administered. By means of another example, one or more molecules as taught herein may be co-administered with a pharmaceutical agent that is not a molecule as envisaged herein. For example, where the molecules as taught herein have anti-cancer properties, they may be combined with known anti-cancer therapy or therapies, such as for example surgery, radiotherapy, chemotherapy, biological therapy, or combinations thereof. The term “chemotherapy” as used herein is conceived broadly and generally encompasses treatments using chemical substances or compositions. Chemotherapeutic agents may typically display cytotoxic or cytostatic effects. In certain embodiments, a chemotherapeutic agent may be an alkylating agent, a cytotoxic compound, an anti-metabolite, a plant alkaloid, a terpenoid, a topoisomerase inhibitor, or a combination thereof. The term “biological therapy” as used herein is conceived broadly and generally encompasses treatments using biological substances or compositions, such as biomolecules, or biological agents, such as viruses or cells. In certain embodiments, a biomolecule may be a peptide, polypeptide, protein, nucleic acid, or a small molecule (such as primary metabolite, secondary metabolite, or natural product), or a combination thereof. Examples of suitable biomolecules include without limitation interleukins, cytokines, anti-cytokines, tumor necrosis factor (TNF), cytokine receptors, vaccines, interferons, enzymes, therapeutic antibodies, antibody fragments, antibody-like protein scaffolds, or combinations thereof. Examples of suitable biomolecules include but are not limited to aldesleukine, alemtuzumab, atezolizumab, bevacizumab, blinatumomab, brentuximab vedotine, catumaxomab, cetuximab, daratumumab, denileukin diftitox, denosumab, dinutuximab, elotuzumab, gemtuzumab ozogamicin, ⁹⁰Y-ibritumomab tiuxetan, idarucizumab, interferon A, ipilimumab, necitumumab, nivolumab, obinutuzumab, ofatumumab, olaratumab, panitumumab, pembrolizumab, ramucirumab, rituximab, tasonermin, ¹³¹I-tositumomab, trastuzumab, Ado-trastuzumab emtansine, and combinations thereof. Examples of suitable oncolytic viruses include but are not limited to talimogene laherparepvec. Further categories of anti-cancer therapy include inter alia hormone therapy (endocrine therapy), immunotherapy, and stem cell therapy, which are commonly considered as subsumed within biological therapies. Examples of suitable hormone therapies include but are not limited to tamoxifen; aromatase inhibitors, such as atanastrozole, exemestane, letrozole, and combinations thereof; luteinizing hormone blockers such as goserelin, leuprorelin, triptorelin, and combinations thereof; anti-androgens, such as bicalutamide, cyproterone acetate, flutamide, and combinations thereof; gonadotrophin releasing hormone blockers, such as degarelix; progesterone treatments, such as medroxyprogesterone acetate, megestrol, and combinations thereof; and combinations thereof. The term “immunotherapy” broadly encompasses any treatment that modulates a subject's immune system. In particular, the term comprises any treatment that modulates an immune response, such as a humoral immune response, a cell-mediated immune response, or both. Immunotherapy comprises cell-based immunotherapy in which immune cells, such as T cells and/or dendritic cells, are transferred into the patient. The term also comprises an administration of substances or compositions, such as chemical compounds and/or biomolecules (e.g., antibodies, antigens, interleukins, cytokines, or combinations thereof), that modulate a subject's immune system. Examples of cancer immunotherapy include without limitation treatments employing monoclonal antibodies, for example Fc-engineered monoclonal antibodies against proteins expressed by tumor cells, immune checkpoint inhibitors, prophylactic or therapeutic cancer vaccines, adoptive cell therapy, and combinations thereof. Examples of immune checkpoint targets for inhibition include without limitation PD-1 (examples of PD-1 inhibitors include without limitation pembrolizumab, nivolumab, and combinations thereof), CTLA-4 (examples of CTLA-4 inhibitors include without limitation ipilimumab, tremelimumab, and combinations thereof), PD-L1 (examples of PD-L1 inhibitors include without limitation atezolizumab), LAG3, B7-H3 (CD276), B7-H4, TIM-3, BTLA, A2aR, killer cell immunoglobulin-like receptors (KIRs), IDO, and combinations thereof. Another approach to therapeutic anti-cancer vaccination includes dendritic cell vaccines. The term broadly encompasses vaccines comprising dendritic cells which are loaded with antigen(s) against which an immune reaction is desired. Adoptive cell therapy (ACT) can refer to the transfer of cells, most commonly immune-derived cells, such as in particular cytotoxic T cells (CTLs), back into the same patient or into a new recipient host with the goal of transferring the immunologic functionality and characteristics into the new host. If possible, use of autologous cells helps the recipient by minimizing tissue rejection and graft vs. host disease issues. Various strategies may for example be employed to genetically modify T cells by altering the specificity of the T cell receptor (TCR) for example by introducing new TCR α and β chains with selected peptide specificity. Alternatively, chimeric antigen receptors (CARs) may be used in order to generate immunoresponsive cells, such as T cells, specific for selected targets, such as malignant cells, with a wide variety of receptor chimera constructs having been described. Examples of CAR constructs include without limitation 1) CARs consisting of a single-chain variable fragment of an antibody specific for an antigen, for example comprising a V_{L} linked to a V_{H} of a specific antibody, linked by a flexible linker, for example by a CD8a hinge domain and a CD8a transmembrane domain, to the transmembrane and intracellular signaling domains of either CD3ζ or FcRγ; and 2) CARs further incorporating the intracellular domains of one or more costimulatory molecules, such as CD28, OX40 (CD134), or 4-1BB (CD137) within the endodomain, or even including combinations of such costimulatory endodomains. Stem cell therapies in cancer commonly aim to replace bone marrow stem cells destroyed by radiation therapy and/or chemotherapy, and include without limitation autologous, syngeneic, or allogeneic stem cell transplantation. The stem cells, in particular hematopoietic stem cells, are typically obtained from bone marrow, peripheral blood or umbilical cord blood. Details of administration routes, doses, and treatment regimens of anti-cancer agents are known in the art, for example as described in “Cancer Clinical Pharmacology” (2005) ed. By Jan H. M. Schellens, Howard L. McLeod and David R. Newell, Oxford University Press. In certain embodiments, a combination therapy with any molecule as taught herein with one or more of a MEK inhibitor (e.g. selumetinib or trametinib), a SHP2 inhibitor (e.g., TN0155), an mTOR inhibitor (e.g., rapamycin or a rapamycin derivative (“rapalog”), including sirolimus, temsirolimus (CCI-779), temsirolimus (CCI-779), everolimus (RAD001), and ridaforolimus (AP-23573)) is envisaged. Active components of any combination therapy may be admixed or may be physically separated, and may be administered simultaneously or sequentially in any order.

Any molecule as taught herein may be administered to subjects in any suitable or operable form or format.

For example, the reference to the molecule as intended herein may encompass a given therapeutically useful compound as well as any pharmaceutically acceptable forms of such compound, such as any addition salts, hydrates or solvates of the compound. The term “pharmaceutically acceptable” as used herein inter alia in connection with salts, hydrates, solvates and excipients, is consistent with the art and means compatible with the other ingredients of a pharmaceutical composition and not deleterious to the recipient thereof. Pharmaceutically acceptable acid and base addition salts are meant to comprise the therapeutically active non-toxic acid and base addition salt forms which the compound is able to form. The pharmaceutically acceptable acid addition salts can conveniently be obtained by treating the base form of a compound with an appropriate acid. Appropriate acids comprise, for example, inorganic acids such as hydrohalic acids, e.g. hydrochloric or hydrobromic acid, sulfuric, nitric, phosphoric and the like acids; or organic acids such as, for example, acetic, propanoic, hydroxyacetic, lactic, pyruvic, malonic, succinic (i.e. butanedioic acid), maleic, fumaric, malic, tartaric, citric, methanesulfonic, ethanesulfonic, benzenesulfonic, p-toluenesulfonic, cyclamic, salicylic, p-aminosalicylic, pamoic and the like acids. Conversely said salt forms can be converted by treatment with an appropriate base into the free base form. A compound containing an acidic proton may also be converted into its non-toxic metal or amine addition salt forms by treatment with appropriate organic and inorganic bases. Appropriate base salt forms comprise, for example, the ammonium salts, the alkali and earth alkaline metal salts, e.g. the lithium, sodium, potassium, magnesium, calcium salts and the like, aluminum salts, zinc salts, salts with organic bases, e.g. primary, secondary and tertiary aliphatic and aromatic amines such as methylamine, ethylamine, propylamine, isopropylamine, the four butylamine isomers, dimethylamine, diethylamine, diethanolamine, dipropylamine, diisopropylamine, di-n-butylamine, pyrrolidine, piperidine, morpholine, trimethylamine, triethylamine, tripropylamine, quinuclidine, pyridine, quinoline and isoquinoline; the benzathine, N-methyl-D-glucamine, hydrabamine salts, and salts with amino acids such as, for example, arginine, lysine and the like. Conversely the salt form can be converted by treatment with acid into the free acid form. The term solvate comprises the hydrates and solvent addition forms which the compound is able to form, as well as the salts thereof. Examples of such forms are, e.g., hydrates, alcoholates and the like.

For example, the molecule may be a part of a composition. The term “composition” generally refers to a thing composed of two or more components, and more specifically particularly denotes a mixture or a blend of two or more materials, such as elements, molecules, substances, biological molecules, or microbiological materials, as well as reaction products and decomposition products formed from the materials of the composition. By means of an example, a composition may comprise any molecule as taught herein in combination with one or more other substances. For example, a composition may be obtained by combining, such as admixing, the molecule as taught herein with said one or more other substances. In certain embodiments, the present compositions may be configured as pharmaceutical compositions. Pharmaceutical compositions typically comprise one or more pharmacologically active ingredients (chemically and/or biologically active materials having one or more pharmacological effects) and one or more pharmaceutically acceptable carriers. Compositions as typically used herein may be liquid, semisolid or solid, and may include solutions or dispersions.

Hence, a further aspect provides a pharmaceutical composition comprising any molecule as taught herein. The terms “pharmaceutical composition” and “pharmaceutical formulation” may be used interchangeably. The pharmaceutical compositions as taught herein may comprise in addition to the one or more actives, one or more pharmaceutically or acceptable carriers. Suitable pharmaceutical excipients depend on the dosage form and identities of the active ingredients and can be selected by the skilled person (e.g., by reference to the Handbook of Pharmaceutical Excipients 7^{th} Edition 2012, eds. Rowe et al.).

As used herein, the terms “carrier” or “excipient” are used interchangeably and broadly include any and all solvents, diluents, buffers (such as, e.g., neutral buffered saline, phosphate buffered saline, or optionally Tris-HCl, acetate or phosphate buffers), solubilisers (such as, e.g., Tween® 80, Polysorbate 80), colloids, dispersion media, vehicles, fillers, chelating agents (such as, e.g., EDTA or glutathione), amino acids (such as, e.g., glycine), proteins, disintegrants, binders, lubricants, wetting agents, emulsifiers, sweeteners, colorants, flavourings, aromatisers, thickeners, agents for achieving a depot effect, coatings, antifungal agents, preservatives (such as, e.g., Thimerosal™, benzyl alcohol), antioxidants (such as, e.g., ascorbic acid, sodium metabisulfite), tonicity controlling agents, absorption delaying agents, adjuvants, bulking agents (such as, e.g., lactose, mannitol) and the like. The use of such media and agents for the formulation of pharmaceutical and cosmetic compositions is well known in the art. Acceptable diluents, carriers and excipients typically do not adversely affect a recipient's homeostasis (e.g., electrolyte balance). The use of such media and agents for pharmaceutical active substances is well known in the art. Such materials should be non-toxic and should not interfere with the activity of the actives. Acceptable carriers may include biocompatible, inert or bioabsorbable salts, buffering agents, oligo- or polysaccharides, polymers, viscosity-improving agents, preservatives and the like. One exemplary carrier is physiologic saline (0.15 M NaCl, pH 7.0 to 7.4). Another exemplary carrier is 50 mM sodium phosphate, 100 mM sodium chloride.

The precise nature of the carrier or other material will depend on the route of administration. For example, the pharmaceutical composition may be in the form of a parenterally acceptable aqueous solution, which is pyrogen-free and has suitable pH, isotonicity and stability.

The pharmaceutical formulations may comprise pharmaceutically acceptable auxiliary substances as required to approximate physiological conditions, such as pH adjusting and buffering agents, preservatives, complexing agents, tonicity adjusting agents, wetting agents and the like, for example, sodium acetate, sodium lactate, sodium phosphate, sodium hydroxide, hydrogen chloride, benzyl alcohol, parabens, EDTA, sodium oleate, sodium chloride, potassium chloride, calcium chloride, sorbitan monolaurate, triethanolamine oleate, etc. Preferably, the pH value of the pharmaceutical formulation is in the physiological pH range, such as particularly the pH of the formulation is between about 5 and about 9.5, more preferably between about 6 and about 8.5, even more preferably between about 7 and about 7.5.

Illustrative, non-limiting carriers for use in formulating the pharmaceutical compositions include, for example, oil-in-water or water-in-oil emulsions, aqueous compositions with or without inclusion of organic co-solvents suitable for intravenous (IV) use, liposomes or surfactant-containing vesicles, microspheres, microbeads and microsomes, powders, tablets, capsules, suppositories, aqueous suspensions, aerosols, and other carriers apparent to one of ordinary skill in the art. Liposomes are artificial membrane vesicles which are useful as delivery vehicles in vitro and in vivo. These formulations may have net cationic, anionic or neutral charge characteristics and are useful characteristics with in vitro, in vivo and ex vivo delivery methods. It has been shown that large unilamellar vesicles (LUV), which range in size from 0.2-4.0 PHI.m can encapsulate a substantial percentage of an aqueous buffer containing large macromolecules. The composition of the liposome is usually a combination of phospholipids, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations.

Pharmaceutical compositions as intended herein may be formulated for essentially any route of administration, such as without limitation, oral administration (such as, e.g., oral ingestion or inhalation), intranasal administration (such as, e.g., intranasal inhalation or intranasal mucosal application), parenteral administration (such as, e.g., subcutaneous, intravenous (I.V.), intramuscular, intraperitoneal or intrasternal injection or infusion), transdermal or transmucosal (such as, e.g., oral, sublingual, intranasal) administration, topical administration, rectal, vaginal or intra-tracheal instillation, and the like. In this way, the therapeutic effects attainable by the methods and compositions can be, for example, systemic, local, tissue-specific, etc., depending of the specific needs of a given application.

For example, for oral administration, pharmaceutical compositions may be formulated in the form of pills, tablets, lacquered tablets, coated (e.g., sugar-coated) tablets, granules, hard and soft gelatin capsules, aqueous, alcoholic or oily solutions, syrups, emulsions or suspensions. In an example, without limitation, preparation of oral dosage forms may be is suitably accomplished by uniformly and intimately blending together a suitable amount of the agent as disclosed herein in the form of a powder, optionally also including finely divided one or more solid carrier, and formulating the blend in a pill, tablet or a capsule. Exemplary but non-limiting solid carriers include calcium phosphate, magnesium stearate, talc, sugars (such as, e.g., glucose, mannose, lactose or sucrose), sugar alcohols (such as, e.g., mannitol), dextrin, starch, gelatin, cellulose, polyvinylpyrrolidine, low melting waxes and ion exchange resins. Compressed tablets containing the pharmaceutical composition can be prepared by uniformly and intimately mixing the agent as disclosed herein with a solid carrier such as described above to provide a mixture having the necessary compression properties, and then compacting the mixture in a suitable machine to the shape and size desired. Moulded tablets maybe made by moulding in a suitable machine, a mixture of powdered compound moistened with an inert liquid diluent. Suitable carriers for soft gelatin capsules and suppositories are, for example, fats, waxes, semisolid and liquid polyols, natural or hardened oils, etc.

For example, for oral or nasal aerosol or inhalation administration, pharmaceutical compositions may be formulated with illustrative carriers, such as, e.g., as in solution with saline, polyethylene glycol or glycols, DPPC, methylcellulose, or in mixture with powdered dispersing agents, further employing benzyl alcohol or other suitable preservatives, absorption promoters to enhance bioavailability, fluorocarbons, and/or other solubilising or dispersing agents known in the art. Suitable pharmaceutical formulations for administration in the form of aerosols or sprays are, for example, solutions, suspensions or emulsions of the agents as taught herein or their physiologically tolerable salts in a pharmaceutically acceptable solvent, such as ethanol or water, or a mixture of such solvents. If required, the formulation can also additionally contain other pharmaceutical auxiliaries such as surfactants, emulsifiers and stabilizers as well as a propellant. Illustratively, delivery may be by use of a single-use delivery device, a mist nebuliser, a breath-activated powder inhaler, an aerosol metered-dose inhaler (MDI) or any other of the numerous nebuliser delivery devices available in the art. Additionally, mist tents or direct administration through endotracheal tubes may also be used.

Examples of carriers for administration via mucosal surfaces depend upon the particular route, e.g., oral, sublingual, intranasal, etc. When administered orally, illustrative examples include pharmaceutical grades of mannitol, starch, lactose, magnesium stearate, sodium saccharide, cellulose, magnesium carbonate and the like, with mannitol being preferred. When administered intranasally, illustrative examples include polyethylene glycol, phospholipids, glycols and glycolipids, sucrose, and/or methylcellulose, powder suspensions with or without bulking agents such as lactose and preservatives such as benzalkonium chloride, EDTA. In a particularly illustrative embodiment, the phospholipid 1,2 dipalmitoyl-sn-glycero-3-phosphocholine (DPPC) is used as an isotonic aqueous carrier at about 0.01-0.2% for intranasal administration of the compound of the subject invention at a concentration of about 0.1 to 3.0 mg/ml.

For example, for parenteral administration, pharmaceutical compositions may be advantageously formulated as solutions, suspensions or emulsions with suitable solvents, diluents, solubilisers or emulsifiers, etc. Suitable solvents are, without limitation, water, physiological saline solution, PBS, Ringer's solution, dextrose solution, or Hank's solution, or alcohols, e.g. ethanol, propanol, glycerol, in addition also sugar solutions such as glucose, invert sugar, sucrose or mannitol solutions, or alternatively mixtures of the various solvents mentioned. The injectable solutions or suspensions may be formulated according to known art, using suitable non-toxic, parenterally-acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid. The agents and pharmaceutically acceptable salts thereof of the invention can also be lyophilised and the lyophilisates obtained used, for example, for the production of injection or infusion preparations. For example, one illustrative example of a carrier for intravenous use includes a mixture of 10% USP ethanol, 40% USP propylene glycol or polyethylene glycol 600 and the balance USP Water for Injection (WFI). Other illustrative carriers for intravenous use include 10% USP ethanol and USP WFI; 0.01-0.1% triethanolamine in USP WFI; or 0.01-0.2% dipalmitoyl diphosphatidylcholine in USP WFI; and 1-10% squalene or parenteral vegetable oil-in-water emulsion. Illustrative examples of carriers for subcutaneous or intramuscular use include phosphate buffered saline (PBS) solution, 5% dextrose in WFI and 0.01-0.1% triethanolamine in 5% dextrose or 0.9% sodium chloride in USP WFI, or a 1 to 2 or 1 to 4 mixture of 10% USP ethanol, 40% propylene glycol and the balance an acceptable isotonic solution such as 5% dextrose or 0.9% sodium chloride; or 0.01-0.2% dipalmitoyl diphosphatidylcholine in USP WFI and 1 to 10% squalene or parenteral vegetable oil-in-water emulsions.

Where aqueous formulations are preferred, such may comprise one or more surfactants. For example, the composition can be in the form of a micellar dispersion comprising at least one suitable surfactant, e.g., a phospholipid surfactant. Illustrative examples of phospholipids include diacyl phosphatidyl glycerols, such as dimyristoyl phosphatidyl glycerol (DPMG), dipalmitoyl phosphatidyl glycerol (DPPG), and distearoyl phosphatidyl glycerol (DSPG), diacyl phosphatidyl cholines, such as dimyristoyl phosphatidylcholine (DPMC), dipalmitoyl phosphatidylcholine (DPPC), and distearoyl phosphatidylcholine (DSPC); diacyl phosphatidic acids, such as dimyristoyl phosphatidic acid (DPMA), dipahnitoyl phosphatidic acid (DPPA), and distearoyl phosphatidic acid (DSPA); and diacyl phosphatidyl ethanolamines such as dimyristoyl phosphatidyl ethanolamine (DPME), dipalmitoyl phosphatidyl ethanolamine (DPPE) and distearoyl phosphatidyl ethanolamine (DSPE). Typically, a surfactant:active substance molar ratio in an aqueous formulation will be from about 10:1 to about 1:10, more typically from about 5:1 to about 1:5, however any effective amount of surfactant may be used in an aqueous formulation to best suit the specific objectives of interest.

When rectally administered in the form of suppositories, these formulations may be prepared by mixing the compounds according to the invention with a suitable non-irritating excipient, such as cocoa butter, synthetic glyceride esters or polyethylene glycols, which are solid at ordinary temperatures, but liquidify and/or dissolve in the rectal cavity to release the drug.

Suitable carriers for microcapsules, implants or rods are, for example, copolymers of glycolic acid and lactic acid.

One skilled in this art will recognise that the above description is illustrative rather than exhaustive. Indeed, many additional formulations techniques and pharmaceutically-acceptable excipients and carrier solutions are well-known to those skilled in the art, as is the development of suitable dosing and treatment regimens for using the particular compositions described herein in a variety of treatment regimens.

The dosage or amount of the molecules as taught herein, optionally in combination with one or more other active compounds to be administered, depends on the individual case and is, as is customary, to be adapted to the individual circumstances to achieve an optimum effect. Thus, the unit dose and regimen depend on the nature and the severity of the disorder to be treated, and also on factors such as the species of the subject, the sex, age, body weight, general health, diet, mode and time of administration, immune status, and individual responsiveness of the human or animal to be treated, efficacy, metabolic stability and duration of action of the compounds used, on whether the therapy is acute or chronic or prophylactic, or on whether other active compounds are administered in addition to the agent of the invention. In order to optimize therapeutic efficacy, the molecule as taught herein can be first administered at different dosing regimens. Typically, levels of the molecule in a tissue can be monitored using appropriate screening assays as part of a clinical testing procedure, e.g., to determine the efficacy of a given treatment regimen. The frequency of dosing is within the skills and clinical judgement of medical practitioners (e.g., doctors, veterinarians or nurses). Typically, the administration regime is established by clinical trials which may establish optimal administration parameters. However, the practitioner may vary such administration regimes according to the one or more of the aforementioned factors, e.g., subject's age, health, weight, sex and medical status. The frequency of dosing can be varied depending on whether the treatment is prophylactic or therapeutic.

Toxicity and therapeutic efficacy of the molecules as described herein or pharmaceutical compositions comprising the same can be determined by known pharmaceutical procedures in, for example, cell cultures or experimental animals. These procedures can be used, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Pharmaceutical compositions that exhibit high therapeutic indices are preferred. While pharmaceutical compositions that exhibit toxic side effects can be used, care should be taken to design a delivery system that targets such compounds to the site of affected tissue in order to minimize potential damage to normal cells (e.g., non-target cells) and, thereby, reduce side effects.

The data obtained from the cell culture assays and animal studies can be used in formulating a range of dosage for use in appropriate subjects. The dosage of such pharmaceutical compositions lies generally within a range of circulating concentrations that include the ED50 with little or no toxicity. The dosage may vary within this range depending upon the dosage form employed and the route of administration utilized. For a pharmaceutical composition used as described herein, the therapeutically effective dose can be estimated initially from cell culture assays. A dose can be formulated in animal models to achieve a circulating plasma concentration range that includes the IC50 (i.e., the concentration of the pharmaceutical composition which achieves a half-maximal inhibition of symptoms) as determined in cell culture. Such information can be used to more accurately determine useful doses in humans. Levels in plasma can be measured, for example, by high performance liquid chromatography.

Without limitation, depending on the type and severity of the disease, a typical dosage (e.g., a typical daily dosage or a typical intermittent dosage, e.g., a typical dosage for every two days, every three days, every four days, every five days, every six days, every week, every 1.5 weeks, every two weeks, every three weeks, every month, or other) of the molecules as taught herein may range from about 10 μg/kg to about 100 mg/kg body weight of the subject, per dose, depending on the factors mentioned above, e.g., may range from about 100 μg/kg to about 100 mg/kg body weight of the subject, per dose, or from about 200 μg/kg to about 75 mg/kg body weight of the subject, per dose, or from about 500 μg/kg to about 50 mg/kg body weight of the subject, per dose, or from about 1 mg/kg to about 25 mg/kg body weight of the subject, per dose, or from about 1 mg/kg to about 10 mg/kg body weight of the subject, per dose, e.g., may be about 100 μg/kg, about 200 μg/kg, about 300 μg/kg, about 400 μg/kg, about 500 μg/kg, about 600 μg/kg, about 700 μg/kg, about 800 μg/kg, about 900 μg/kg, about 1.0 mg/kg, about 2.0 mg/kg, about 5.0 mg/kg, about 10 mg/kg, about 15 mg/kg, about 20 mg/kg, about 30 mg/kg, about 40 mg/kg, about 50 mg/kg, about 75 mg/kg, or about 100 mg/kg body weight of the subject, per dose.

In particular embodiments, the molecule as taught herein is administered using a sustained delivery system, such as a (partly) implanted sustained delivery system. Skilled person will understand that such a sustained delivery system may comprise a reservoir for holding the agent as taught herein, a pump and infusion means (e.g., a tubing system).

As already discussed, further aspects provide an in vitro method for downregulating the amount or biological activity of a mutant or variant form of a protein in a cell expressing, preferably endogenously expressing, the mutant or variant form of the protein, the method comprising contacting the cell with a non-naturally occurring molecule capable of downregulating the amount or biological activity of the mutant or variant form of the protein, wherein:
a) the protein comprises a β-aggregation prone region (APR) and said APR is modified by the mutation or variation in the mutant or variant form of the protein; or
b) the mutation or variation introduces a de novo APR in the mutant or variant form of the protein not present in the protein;
and wherein the molecule is configured to specifically target the APR in the mutant or variant form of the protein as taught herein.

The term “in vitro” generally denotes outside, or external to, a body, e.g., an animal or human body. Cells can be isolated, maintained and propagated in vitro using cell isolation and culture techniques, materials and disposables well-known in the art. The term “contact” or “contacting” as used herein means bringing one or more first components (such as one or more molecules, biological entities, cells, or materials) together with one or more second components (such as one or more molecules, biological entities, cells, or materials) in such a manner that the first component(s) can—if capable thereof—bind or modulate the second component(s) or that the second component(s) can—if capable thereof—bind or modulate the first component(s). The term “contacting” may depending on the context be synonymous with “exposing”, “incubating”, “mixing”, “reacting”, or the like.

In certain embodiments, the cell may be a bacterial cell, a fungal cell, including a yeast cell or a mould cell, a protist cell, a plant cell, or an animal cell, such as an insect cell, a warm-blooded animal cell, a vertebrate cell, a higher animal cell, a non-human mammal cell or a human cell.

Further aspects provide a method for downregulating the amount or biological activity of a mutant or variant form of a protein in an organism expressing, preferably endogenously expressing, the mutant or variant form of the protein, the method comprising administering to the organism a non-naturally occurring molecule capable of downregulating the amount or biological activity of the mutant or variant form of the protein, wherein:
a) the protein comprises a β-aggregation prone region (APR) and said APR is modified by the mutation or variation in the mutant or variant form of the protein; or
b) the mutation or variation introduces a de novo APR in the mutant or variant form of the protein not present in the protein;
and wherein the molecule is configured to specifically target the APR in the mutant or variant form of the protein as taught herein.

In certain embodiments, the organism may be a bacterium, a fungus, including yeast or mould, a plant, or an animal. Therapeutic uses of the molecules in humans and non-human animals are discussed in more detail elsewhere in the specification, while in certain embodiments, the methods may be non-therapeutic, e.g., the methods may be ones that are not for treatment of the human or animal body by surgery or therapy. In certain preferred embodiments, the organism may be a plant. In certain preferred embodiments, the organism may be a non-vertebrate or a lower animal.

The term “plant” as used herein encompasses whole plants, ancestors and progeny of the plants and plant parts, including seeds, shoots, stems, leaves, roots (including tubers), flowers, and tissues and organs, wherein such plants or plant parts express the mutant or variant protein form. Also encompassed by the terms “plant cell” or “plant” may be suspension cultures, callus tissue, embryos, meristematic regions, gametophytes, sporophytes, pollen and microspores, wherein these express the mutant or variant protein form. Plants that are particularly useful in the methods of the invention include in particular monocotyledonous and dicotyledonous plants including fodder or forage legumes, ornamental plants, food crops, trees or shrubs.

The aforementioned concepts are illustrated and further explained by the following specific example. RAS proteins belong to small GTPase class of proteins and are involved in cytoplasmic signal transduction pathways regulating diverse normal cellular processes, such as cell growth and division, differentiation and survival. RAS GTPases cycle between the GDP-bound inactive and GTP-bound active states with the help of guanine nucleotide exchange factors (GEFs) that promote activation and GTPase-activating proteins (GAPs) that inactivate RAS by catalysing GTP hydrolysis. Once activated, RAS-GTP binds to and activates a spectrum of downstream effectors with distinct catalytic functions. The three human RAS genes (Kirsten rat sarcoma viral oncogene homolog (KRAS), annotated under U.S. government's National Center for Biotechnology Information (NCBI) Genbank (http://www.ncbi.nlm.nih.gov/) Gene ID no. 3845, neuroblastoma RAS viral oncogene homolog (NRAS), Gene ID no. 4893, and Harvey rat sarcoma viral oncogene homolog (HRAS), Gene ID no. 3265) encode four RAS proteins, with two KRAS isoforms that arise from alternative RNA splicing of the KRAS transcript (KRAS4A and KRAS4B).

A human wild-type KRAS4A isoform amino acid sequence may be as annotated under Genbank accession no: NP_203524.1 or Swissprot/Uniprot (http://www.uniprot.org/) accession no: P01116-1 (v1), the NP_203524.1 sequence reproduced here below:

(SEQ ID NO: 1)
MTEYKLVVVGAGGVGKSALTIQLIQNHFVDEYDPTIEDSYRKQVVIDGE
TCLLDILDTAGQEEYSAMRDQYMRTGEGFLCVFAINNTKSFEDIHHYRE
QIKRVKDSEDVPMVLVGNKCDLPSRTVDTKQAQDLARSYGIPFIETSAK
TRQRVEDAFYTLVREIRQYRLKKISKEEKTPGCVKIKKCIIM

Certain mutations in RAS genes can lead to the production of permanently activated RAS proteins, leading to active intracellular signalling even in the absence of incoming signals, which can ultimately result in or contribute to neoplastic transformation of cells expressing such mutated RAS proteins. Gain-of-function missense mutations in RAS genes (more than 130 different missense mutations have been reported in RAS genes) are found in about 27% of all human cancers and up to 90% in certain types of cancer, validating mutant RAS genes as very common if not the most common oncogenes driving tumour initiation and maintenance. In human cancers, KRAS is the predominantly mutated RAS isoform (85%), whereas HRAS (4%) and NRAS (11%) are less frequently mutated. Moreover, 98% of the mutations are found at one of three missense-mutation hotspots: G12 (with G12C, G12D, G12S, and G12V mutations being among the most frequent at G12), G13 (with G13C, G13D, G13R, G13S, and G13V mutations being among the most frequent at G13) and Q61 (with Q61H, Q61K, Q61L, and Q61R mutations being among the most frequent at Q61). Conventionally, mutant RAS is considered to be defective in GAP-mediated GTP hydrolysis, which results in an accumulation of constitutively active GTP-bound RAS in cells. See Hobbs et al. J Cell Sci. 2016, vol. 129, 1287-92.

Human RAS proteins are predicted to contain 5 APR regions of at least 5 amino acids (see Table 3). The most N-terminal APR (TEYKLVVVGAG, SEQ ID NO: 2) is C-terminally delineated by G12 (underlined) in the wild-type proteins. However, certain G12 missense mutations, such as particularly G12V, G12C, G12A, or G12S enlarge this APR such that the APRs in the respective RAS mutants include not only the mutated residue at position 12 but additionally one or more subsequent residues. Further, certain G13 missense mutations, such as particularly G13V, G13C, or G13S, enlarge this APR such that the APRs in the respective RAS mutants include not only the glycine at position 12 but additionally the mutated residue at position 13 and optionally one or more subsequent residues.

Accordingly, this APR is predicted to span positions 2-15 and display the sequence TEYKLVVVGAVGVG (SEQ ID NO: 3) in the G12V RAS mutant; to span positions 2-14 and display the sequence TEYKLVVVGACGV (SEQ ID NO: 4) in the G12C RAS mutant; to span positions 2-14 and display the sequence TEYKLVVVGAAGV (SEQ ID NO: 5) in the G12A RAS mutant; and to span positions 2-13 and display the sequence TEYKLVVVGASG (SEQ ID NO: 6) in the G12S RAS mutant; to span positions 2-14 and display the sequence TEYKLVVVGAGCV (SEQ ID NO: 7) in the G13C RAS mutant; to span positions 2-15 and display the sequence TEYKLVVVGAGVVG (SEQ ID NO: 8) in the G13V RAS mutant; and to span positions 2-13 and display the sequence TEYKLVVVGAGS (SEQ ID NO: 9) in the G13S RAS mutant. Additionally, at least some mutations at G12 or G13 of human RAS, such as in particular the G12V or G13V mutations, also significantly increase the predicted aggregation propensity of the corresponding APR.

Having recognised the presence of such altered APR profiles in G12 or G13 mutant human RAS proteins, the inventors investigated and presently teach molecules which exploit these differences by specifically targeting the altered APRs in the G12 or G13 mutant RAS proteins, but not the corresponding unaltered APR in wild-type RAS, for an intermolecular n-sheet interaction that allows to downregulate the G12 or G13 mutant RAS proteins. Without wishing to be limited to any hypothesis or theory, the data presented herein suggests that this downregulation is likely due to the ability of the molecules to induce specific co-aggregation with the G12 or G13 mutant RAS proteins, which decreases their solubility, sequesters them into aggregates or inclusion bodies (which may be subject to degradation by cellular machinery), and in effect reduces the amount of the G12 or G13 mutant RAS proteins that remain available for intracellular signalling. It shall be understood that once a molecule induced or commenced the aggregation of its target G12 or G13 mutant RAS protein, the so-aggregated RAS can itself acquire the capacity to facilitate or drive the inclusion of additional soluble G12 or G13 mutant RAS protein into the aggregates, i.e., the existing RAS aggregates can function as ‘seeds’ for further aggregation of the protein and growth of the aggregates. The molecules do not display a comparable or equivalent induction of co-aggregation with and downregulation of wild-type RAS. This may for instance mean that even if some intermolecular n-sheet formation were to occur between the molecules and wild-type RAS, the consequences of this will be comparatively negligible and the molecules will not observably downregulate wild-type RAS or will not downregulate wild-type RAS to an extent where such downregulation would detrimentally diminish intracellular signalling by wild-type RAS.

Hence, certain molecules embodying the principles of the present invention are capable of downregulating, decreasing the solubility and/or inducing aggregation or inclusion body formation of a G12 mutant human RAS protein and substantially not of wild-type human RAS protein, wherein the molecule comprises a β-aggregating sequence comprising at least 6, such as 6, 7, 8, 9, or 10, contiguous amino acids of the amino acid sequence: a) TEYKLVVVGAVGVG (SEQ ID NO: 3); or b) TEYKLVVVGACGV (SEQ ID NO: 4); or c) TEYKLVVVGAAGV (SEQ ID NO: 5); or d) TEYKLVVVGASG (SEQ ID NO: 6), including the amino acid at position 11 of the respective sequences. In certain molecules embodying the principles of the present invention are capable of downregulating, decreasing the solubility and/or inducing aggregation or inclusion body formation of a G12 mutant human RAS protein and substantially not of wild-type human RAS protein, wherein the molecule comprises a β-aggregating sequence comprising at least 6, such as 6, 7, 8, 9, or 10 (or the maximum), contiguous amino acids of the amino acid sequence: a) LVVVGAVGVG (SEQ ID NO: 10); or b) LVVVGACGV (SEQ ID NO: 11); or c) LVVVGAAGV (SEQ ID NO: 12); or d) LVVVGASG (SEQ ID NO: 13), including the amino acid at position 7 of the respective sequences. In connection with G12C RAS, the inclusion of an unprotected cysteine in the molecule may be less opportune due to the presence of the reactive —SH group in the cysteine residue. Accordingly, molecules directed against G12C RAS, and more generally against any APR containing cysteine(s), may contain another amino acid, such as serine, at that position, or may contain a cysteine at that position that is otherwise protected, for example by a protective group (e.g., a p-methylbenzyl group, a diphenylmethyl group, a p-methoxybenzyl group, or an acetamidomethyl group), or by reacting its —SH group with the —SH group of another cysteine in the same molecule or between two molecules (disulphide bridge). Hence, in certain embodiments, in a molecule directed to G12C mutant human RAS, the amino acid of the molecule stretch that corresponds to position 12 of the G12C RAS would be L-serine or D-serine or a serine analogue, preferably L-serine. In certain other embodiments, in a molecule directed to G12C mutant human RAS, the amino acid of the molecule stretch that corresponds to position 12 of the G12C RAS would be L-cysteine or D-cysteine or a cysteine analogue, preferably L-cysteine, having its —SH group protected by a protective group or participating in a disulphide bridge.

Certain molecules embodying the principles of the present invention are capable of downregulating, decreasing the solubility and/or inducing aggregation or inclusion body formation of a G13 mutant human RAS protein and substantially not of wild-type human RAS protein, wherein the molecule comprises a β-aggregating sequence comprising at least 6, such as 6, 7, 8, 9, or 10, contiguous amino acids of the amino acid sequence: a) TEYKLVVVGAGCV (SEQ ID NO: 7); or b) TEYKLVVVGAGVVG (SEQ ID NO: 8); or c) TEYKLVVVGAGS (SEQ ID NO: 9); including the amino acid at position 12 of the respective sequences. Certain molecules embodying the principles of the present invention are capable of downregulating, decreasing the solubility and/or inducing aggregation or inclusion body formation of a G13 mutant human RAS protein and substantially not of wild-type human RAS protein, wherein the molecule comprises a β-aggregating sequence comprising at least 6, such as 6, 7, 8, 9, or 10 (or the maximum), contiguous amino acids of the amino acid sequence: a) LVVVGAGCV (SEQ ID NO: 14); or b) LVVVGAGVVG (SEQ ID NO: 15); or c) LVVVGAGS (SEQ ID NO: 16); including the amino acid at position 8 of the respective sequences.

For example, a G12 or G13 mutant RAS targeting molecule may be represented as comprising, consisting essentially of or consisting of the structure:
a) Gate-Pept-Gate;
b) Linker-Gate-Pept-Gate;
c) Gate-Pept-Gate-Linker;
d) Linker-Gate-Pept-Gate-Linker;
e) Gate-Pept-Gate-(Linker)-Gate-Pept-Gate;
f) Linker-Gate-Pept-Gate-(Linker)-Gate-Pept-Gate;
g) Gate-Pept-Gate-(Linker)-Gate-Pept-Gate-Linker;
h) Linker-Gate-Pept-Gate-(Linker)-Gate-Pept-Gate-Linker;
i) Gate-Pept-Gate-(Linker)-Gate-Pept-Gate-(Linker)-Gate-Pept-Gate;
j) Linker-Gate-Pept-Gate-(Linker)-Gate-Pept-Gate-(Linker)-Gate-Pept-Gate;
k) Gate-Pept-Gate-(Linker)-Gate-Pept-Gate-(Linker)-Gate-Pept-Gate-Linker; or
l) Linker-Gate-Pept-Gate-(Linker)-Gate-Pept-Gate-(Linker)-Gate-Pept-Gate-Linker;
wherein “Gate”, “Pept”, and “Linker” denote peptide elements bound to the adjacent peptide element(s) by peptide bond(s), wherein left-to-right order of the peptide elements signifies their N- to C-terminal organisation in the peptide;
wherein “Pept” (directed against G12 mutant RAS) is each independently a β-aggregating sequence comprising at least 6, such as 6, 7, 8, 9, or 10 (or the maximum), contiguous amino acids of the amino acid sequence: LVVVGAVGVG (SEQ ID NO: 10), or LVVVGACGV (SEQ ID NO: 11), or LVVVGAAGV (SEQ ID NO: 12), or LVVVGASG (SEQ ID NO: 13), including the amino acid at position 7 of the respective sequences, optionally wherein any one or more or all of the recited amino acids is or are replaced by its or their D-isomer(s) or by its or their analogue(s), including L- and D-isomers of such analogue(s) (as explained elsewhere in this specification, the cysteine may, in any “Pept” denoted as containing cysteine, be swapped for a serine or protected by a suitable protective group or a disulphide bridge);
or wherein “Pept” (directed against G13 mutant RAS) is each independently a β-aggregating sequence comprising at least 6, such as 6, 7, 8, 9, or 10 (or the maximum), contiguous amino acids of the amino acid sequence: LVVVGAGCV (SEQ ID NO: 14), or LVVVGAGVVG (SEQ ID NO: 15), or LVVVGAGS (SEQ ID NO: 16), including the amino acid at position 8 of the respective sequences, optionally wherein any one or more or all of the recited amino acids is or are replaced by its or their D-isomer(s) or by its or their analogue(s), including L- and D-isomers of such analogue(s) (as explained elsewhere in this specification, the cysteine may, in any “Pept” denoted as containing cysteine, be swapped for a serine or protected by a suitable protective group or a disulphide bridge);
wherein “Gate” is each independently lysine (K) or D-lysine or D- or L-lysine analogue (preferably lysine), arginine (R) or D-arginine or D- or L-arginine analogue (preferably arginine), aspartic acid (D) or D-aspartic acid or D- or L-aspartic acid analogue (preferably aspartic acid), glutamic acid (E) or D-glutamic acid or D- or L-glutamic acid analogue (preferably glutamic acid), KK, KKK, KKKK (SEQ ID NO: 45), RR, RRR, RRRR (SEQ ID NO: 46), DD, DDD, DDDD (SEQ ID NO: 47), EE, EEE, EEEE (SEQ ID NO: 48), KR, RK, KKR, KRK, RKK, RRK, RKR, KRR, KRKR (SEQ ID NO: 49), KRRK (SEQ ID NO: 50), RKKR (SEQ ID NO: 51), DE, ED, DDE, DED, EED, EED, EDE, DEE, DEDE (SEQ ID NO: 52), DEED (SEQ ID NO: 53), or EDDE (SEQ ID NO: 54), optionally wherein any one or more or all of the recited amino acids is or are replaced by its or their D-isomer(s) or by its or their analogue(s), including L- and D-isomers of such analogue(s); and wherein the inclusion of the word “Linker” in parentheses denotes that the linker, each independently, may be absent or is preferably present, and wherein “Linker” is each independently glycine (G) or D- or L-glycine analogue (preferably glycine), serine (S) or D-serine or D- or L-serine analogue (preferably serine), proline (P) or D-proline or D- or L-proline analogue (preferably proline), GG, GGG, GGGG (SEQ ID NO: 55), SS, SSS, SSSS (SEQ ID NO: 56), GS, SG, GGS, GSG, SGG, SSG, SGS, SSG, GGGS (SEQ ID NO: 57), GGSG (SEQ ID NO: 58), GSGG (SEQ ID NO: 59), SGGG (SEQ ID NO: 60), GGSS (SEQ ID NO: 61), GSSG (SEQ ID NO: 62), SSGG (SEQ ID NO: 63), GSGS (SEQ ID NO: 70), SGSG (SEQ ID NO: 64), GSGSG (SEQ ID NO: 65), SGSGS (SEQ ID NO: 66), PP, PPP, or PPPP (SEQ ID NO: 67), optionally wherein any one or more or all of the recited amino acids is or are replaced by its or their D-isomer(s) or by its or their analogue(s), including L- and D-isomers of such analogue(s).

In such peptides, the N-terminal amino acid may be modified such as acetylated and/or the C-terminal amino acid may be modified such as amidated. In such peptides, D-amino acid(s) and or amino acid analogue(s) can be incorporated as long as their incorporation is compatible with the formation of the intermolecular beta-sheet as taught herein.

For example, a G12V mutant RAS targeting molecule may comprise, consist essentially of or consist of a peptide of the amino acid sequence:

| | a) |
|---|---|
| (SEQ ID NO: 17) | |
| | KVVVGAVKGSKVVVGAVK; |
| | or |
| | b) |
| (SEQ ID NO: 18) | |
| | KLVVVGAVKGSKLVVVGAVK; |
| | or |
| | c) |
| (SEQ ID NO: 19) | |
| | KVVVGAVGKGSKVVVGAVGK; |
| | or |
| | d) |
| (SEQ ID NO: 20) | |
| | KVVVGAVGVGKGSKVVVGAVGVGK; |

optionally wherein the amino acid sequence comprises one or more D-amino acids and/or analogues of one or more of its amino acids, optionally wherein the N-terminal amino acid is acetylated and/or the C-terminal amino acid is amidated.

In certain particularly preferred embodiments, the molecule comprises, consists essentially of or consists of a peptide of the amino acid sequence as shown in Table 7, such as SEQ ID NO: 76, 77-78, 80-95, 97, or 99-100, optionally wherein the amino acid sequence comprises one or more D-amino acids and/or analogues of one or more of its amino acids, optionally wherein the N-terminal amino acid is acetylated and/or the C-terminal amino acid is amidated. Hence, in certain particularly preferred embodiments, the molecule comprises, consists essentially of or consists of a peptide of the amino acid sequence:

| | a) |
|---|---|
| (SEQ ID NO: 79) | |
| | [Dap]LSVFAIKGSKLSVFAI[Dap]; |
| | or |
| | b) |
| (SEQ ID NO: 80) | |
| | [Dap]VVVGAVKGSKVVVGAV[Dap]; |
| | or |
| | c) |
| (SEQ ID NO: 81) | |
| | [Dap]VVVGAVGKGSKVVVGAVG[Dap]; |
| | or |
| | d) |
| (SEQ ID NO: 82) | |
| | [Dap]VVVGAVGVGKGSKVVVGAVGVG[Dap]; |
| | or |
| | e) |
| (SEQ ID NO: 83) | |
| | [Cit]VVVGAVKGSKVVVGAVK; |
| | or |
| | f) |
| (SEQ ID NO: 84) | |
| | KVVVGAV[Cit]GSKVVVGAVK; |
| | or |
| | g) |
| (SEQ ID NO: 85) | |
| | AVVVGAVKGSKVVVGAVK; |
| | or |
| | h) |
| (SEQ ID NO: 86) | |
| | KVVVGAVAGSKVVVGAVK; |
| | or |
| | i) |
| (SEQ ID NO: 87) | |
| | KVVVGAVKGSAVVVGAVK; |
| | or |
| | j) |
| (SEQ ID NO: 88) | |
| | KVVVGAVKGSKVVVGAVA; |
| | or |
| | k) |
| (SEQ ID NO: 89) | |
| | AVVVGAVKGSAVVVGAVK; |
| | or |
| | l) |
| (SEQ ID NO: 90) | |
| | KVVVGAVAGSKVVVGAVA; |
| | or |
| | m) |
| (SEQ ID NO: 91) | |
| | AVVVGAVAGSKVVVGAVK; |
| | or |
| | n) |
| (SEQ ID NO: 92) | |
| | KVVVGAVKASKVVVGAVK; |
| | or |
| | o) |
| (SEQ ID NO: 93) | |
| | KVVVGAVKGAKVVVGAVK; |
| | or |
| | p) |
| (SEQ ID NO: 94) | |
| | KVVVGAVGKGFKVVVGAVGK; |
| | or |
| | q) |
| (SEQ ID NO: 95) | |
| | KVVVGAVGKFFKVVVGAVGK; |
| | or |
| | r) |
| (SEQ ID NO: 97) | |
| | KVVVGAVGVGKKVVVGAVGVGK; |

optionally wherein the amino acid sequence comprises one or more D-amino acids and/or analogues of one or more of its amino acids, optionally wherein the N-terminal amino acid is acetylated and/or the C-terminal amino acid is amidated (‘[Dap]’ denotes diaminopimelic acid, ‘[Cit]’ denotes citrulline).

In certain embodiments, the molecule as taught herein is not a peptide consisting of the amino acid sequence KLVVVGAVGV (SEQ ID NO: 101). In certain embodiments, the molecule as taught herein is not a peptide consisting of the amino acid sequence KLVVVGAVGVGKSALTI (SEQ ID NO: 102). In certain embodiments, the molecule as taught herein is not a peptide consisting of the amino acid sequence KLVVVGAVGVGKS (SEQ ID NO: 103).

Such molecules and their effects and uses are also experimentally illustrated in the Examples.

By means of further illustration and without limitation, the following provides examples of known mutations in human genes which alter or add a TANGO-predicted APR in the corresponding mutant proteins.

Examples of Disease Mutations in Oncogenes that Alter Length of Existing APR:

GNAS (Guanine Nucleotide-Binding Protein G(s) Subunit Alpha Isoforms Short, Swissprot/UniProt Acc. No. P63092 Sequence Version 1):

| | Start | N- | | C- | | |
|---|---|---|---|---|---|---|
| | position | ter | APR | ter | Score | Length |
| Mutation | APR | GKs | sequence | GKs | (%) | (aa) |
| WT | 201 | RCR | VLTSGIF | ETK | 10.5573 | 7 |
| R201C | 200 | LRC | CVLTSGIF | ETK | 4.2066 | 8 |
| R201L | 199 | LLR | CLVLTSGI | ETK | 39.345 | 9 |
| | | | F | | | |

The sequences in rows 1-3 of the above table are denoted as SEQ ID NO: 21-23, respectively.

MP2K2 (Dual Specificity Mitogen-Activated Protein Kinase Kinase 2, Swissprot/UniProt Acc. No. P36507 Sequence Version 1):

| | Start | N- | | C- | | |
|---|---|---|---|---|---|---|
| | position | ter | APR | ter | Score | Length |
| Mutation | APR | GKs | sequence | GKs | (%) | (aa) |
| WT | 128 | NSP | YIVGFYGA | DGE | 65.216 | 11 |
| | | | FYS | | | |
| P128L | 126 | ECN | SLYIVGFY | DGE | 69.3177 | 13 |
| | | | GAFYS | | | |

The sequences in rows 1-2 of the above table are denoted as SEQ ID NO: 24-25, respectively.

IDHP (Isocitrate Dehydrogenase [NADP], Mitochondrial, Swissprot/UniProt Acc. No. P48735 Sequence Version 2):

| | Start | N- | | C- | | |
|---|---|---|---|---|---|---|
| | position | ter | APR | ter | Score | Length |
| Mutation | APR | GKs | sequence | GKs | (%) | (aa) |
| WT | 141 | IRN | ILGGTVF | REP | 4.02996 | 7 |
| R140L | 137 | PNG | TILNILGG | REP | 28.1 | 11 |
| | | | TVF | | | |
| R140W | 137 | PNG | TIWNILGG | REP | 23.8732 | 11 |
| | | | TVF | | | |

The sequences in rows 1-3 of the above table are denoted as SEQ ID NO: 26-28, respectively.

ITK (Tyrosine-Protein Kinase ITK/TSK, Swissprot/UniProt Acc. No. Q08881, Sequence Version 1):

| | Start | N- | | C- | | |
|---|---|---|---|---|---|---|
| | position | ter | APR | ter | Score | Length |
| Mutation | APR | GKs | sequence | GKs | (%) | (aa) |
| WT | 29 | KVR | FFVLTKAS | DRH | 26.0231 | 13 |
| | | | LAYFE | | | |
| R29L | 27 | NFK | VLFFVLTK | DRH | 43.8347 | 15 |
| | | | ASLAYFE | | | |
| R29C | 27 | NFK | VCFFVLTK | DRH | 39.996 | 15 |
| | | | ASLAYFE | | | |

The sequences in rows 1-3 of the above table are denoted as SEQ ID NO: 29-31, respectively.

B) Examples of Disease Mutations in Oncogenes that do not Alter Length of APR but Create a Mismatch and/or Alter Score:

BCL2 (Apoptosis Regulator Bcl-2, Swissprot/UniProt Acc. No. P10415, Sequence Version 2):

| | Start | N- | | C- | | |
|---|---|---|---|---|---|---|
| | position | ter | APR | ter | Score | Length |
| Mutation | APR | GKs | sequence | GKs | (%) | (aa) |
| WT | 129 | RGR | FATVV | EEL | 38.5828 | 5 |
| A131V | 129 | RGR | FVTVV | EEL | 90.0278 | 5 |
| A131G | 129 | RGR | FGTVV | EEL | 6.25217 | 5 |

The sequences in rows 1-3 of the above table are denoted as SEQ ID NO: 34-36, respectively.

Examples of Disease Mutations in Oncogenes that Create a De Novo APR:

ERBB2 (Receptor Tyrosine-Protein Kinase erbB-2, Swissprot/UniProt Acc. No. P04626, Sequence Version 1):

| | Start | N- | | C- | | |
|---|---|---|---|---|---|---|
| | position | ter | APR | ter | Score | Length |
| Mutation | APR | GKs | sequence | GKs | (%) | (aa) |
| WT | N/A | N/A | N/A | N/A | N/A | N/A |
| A293V | 288 | EGR | YTFGVSCV | TAC | 1.72107 | 8 |

The sequence in row 2 of the above table is denoted as SEQ ID NO: 37, respectively.

B-RAF (Serine/Threonine-Protein Kinase B-RAF, Swissprot/UniProt Acc. No. P15056, Sequence Version 4):

| | Start | N- | | C- | | |
|---|---|---|---|---|---|---|
| | position | ter | APR | ter | Score | Length |
| Mutation | APR | GKs | sequence | GKS | (%) | (aa) |
| WT | N/A | N/A | N/A | N/A | N/A | N/A |
| G469V | 466 | GSG | SFVTVY | KGK | 47.4044 | 6 |
| G469L | 466 | GSG | SFLTVY | KGK | 31.7404 | 6 |

The sequences in row 2-3 of the above table is denoted as SEQ ID NO: 38-39, respectively.

The present application also provides aspects and embodiments as set forth in the following Statements:

Statement 1. A non-naturally occurring molecule capable of downregulating the amount or biological activity of a mutant or variant form of a protein, wherein:
a) the protein comprises a 3-aggregation prone region (APR) and said APR is modified by the mutation or variation in the mutant or variant form of the protein; or
b) the mutation or variation introduces a de novo APR in the mutant or variant form of the protein not present in the protein;
and wherein the molecule is configured to specifically target the APR in the mutant or variant form of the protein.

Statement 2. The molecule according to Statement 1, wherein the molecule is configured to form an intermolecular beta-sheet with the APR in the mutant or variant form of the protein but substantially not with the APR in the protein.

Statement 3. The molecule according to Statement 1 or 2, wherein the intermolecular beta-sheet involves one or more of the amino acids which differ between the mutant or variant form of the protein and the protein.

Statement 4. The molecule according to any one of Statements 1 to 3, wherein the APR in the mutant or variant form of the protein differs from the APR in the protein in amino acid sequence or aggregation propensity, preferably in amino acid sequence, more preferably in amino acid sequence and aggregation propensity.

Statement 5. The molecule according to Statement 4, wherein the aggregation propensity of the APR in the mutant or variant form of the protein is higher than the aggregation propensity of the APR in the protein.

Statement 6. The molecule according to Statement 4 or 5, wherein:
a) the APR in the mutant or variant form of the protein has a higher proportion of hydrophobic amino acids than the APR in the protein;
b) the APR in the mutant or variant form of the protein has a lower proportion of amino acids that display low beta-sheet forming potential or a propensity to disrupt beta-sheets than the APR in the protein;
c) the APR in the mutant or variant form of the protein has a lower proportion of charged amino acids than the APR in the protein; and/or
d) the APR in the mutant or variant form of the protein is at least one amino acid longer than the APR in the protein, such as two, three or four amino acids longer.

Statement 7. The molecule according to any one of Statements 4 to 6, wherein:
a) the mutation or variation in the mutant or variant form of the protein modifies, such as substitutes, deletes or adds, one or more amino acids within the APR in the protein;
b) the mutation or variation in the mutant or variant form of the protein modifies, such as substitutes, deletes or adds, one or more amino acids within a region of between 1 and 10, preferably between 1 and 4 contiguous amino acids N-terminally adjacent to the APR in the protein, preferably whereby at least one amino acid of said region becomes part of the APR in the mutant or variant form of the protein; and/or
c) the mutation or variation in the mutant or variant form of the protein modifies, such as substitutes, deletes or adds, one or more amino acids within a region of between 1 and 10, preferably between 1 and 4 contiguous amino acids C-terminally adjacent to the APR in the protein, preferably whereby at least one amino acid of said region becomes part of the APR in the mutant or variant form of the protein.

Statement 8. The molecule according to Statement 7, wherein the mutation or variation in said region N- or C-terminally adjacent to the APR in the protein:
a) increases the proportion of hydrophobic amino acids in said region;
b) reduces the proportion of amino acids that display low beta-sheet forming potential or a propensity to disrupt beta-sheets said region; and/or
c) reduces the proportion of charged amino acids in said region.

Statement 9. The molecule according to any one of Statements 1 to 8, wherein the molecule is able to decrease the solubility or to induce the aggregation or inclusion body formation of the mutant or variant form of the protein.

Statement 10. The molecule according to any one of Statements 2 to 9, wherein the molecule comprises an amino acid stretch, preferably a stretch of at least 6 contiguous amino acids, such as a stretch of 6 to 10 contiguous amino acids, which participates in the intermolecular beta-sheet with the APR in the mutant or variant form of the protein.

Statement 11. The molecule according to Statement 10, wherein said stretch comprised by the molecule corresponds to an amino acid stretch, preferably to a stretch of at least 6 contiguous amino acids, such as a stretch of 6 to 10 contiguous amino acids, comprised by the APR in the mutant or variant form of the protein, preferably wherein:
a) the amino acid sequence of the stretch comprised by the molecule is identical to the stretch comprised by the APR;
b) the amino acid sequence of the stretch comprised by the molecule is at least 80% identical to the amino acid sequence of the stretch comprised by the APR;
c) the amino acid sequence of the stretch comprised by the molecule differs from the amino acid sequence of the stretch comprised by the APR by at most 3, preferably at most 2, and more preferably at most 1 amino acid substitutions;
d) the amino acid sequence of the stretch comprised by the molecule displays the degree of sequence identity to the amino acid sequence of the stretch comprised by the APR as set forth in any one of a) to c), and all amino acids of the molecule stretch are L-amino acids;
e) the amino acid sequence of the stretch comprised by the molecule displays the degree of sequence identity to the amino acid sequence of the stretch comprised by the APR as set forth in any one of a) to c), and at least one amino acid of the former stretch is a D-amino acid;
f) the amino acid sequence of the stretch comprised by the molecule displays the degree of sequence identity to the amino acid sequence of the stretch comprised by the APR as set forth in any one of a) to c), and at least one amino acid of the former stretch is replaced by an analogue of the respective amino acid; or
g) the amino acid sequence of the stretch comprised by the molecule displays the degree of sequence identity to the amino acid sequence of the stretch comprised by the APR as set forth in any one of a) to c), and at least one amino acid of the former stretch is a D-amino acid and at least one amino acid of the former stretch is replaced by an analogue of the respective amino acid.

Statement 12. The molecule according to Statement 10 or 11, wherein the molecule comprises two or more, preferably two, said amino acid stretches, which are identical or different.

Statement 13. The molecule according to any one of Statements 10 to 12, wherein the amino acid stretch or stretches are each independently flanked, on each end independently, by one or more amino acids that display low beta-sheet forming potential or a propensity to disrupt beta-sheets.

Statement 14. The molecule according to any one of Statements 10 to 13, wherein the molecule comprises, consists essentially of or consists of the structure:
a) NGK1-P1-CGK1,
b) NGK1-P1-CGK1-Z1-NGK2-P2-CGK2,
c) NGK1-P1-CGK1-Z1-NGK2-P2-CGK2-Z2-NGK3-P3-CGK3, or
d) NGK1-P1-CGK1-Z1-NGK2-P2-CGK2-Z2-NGK3-P3-CGK3-Z3-NGK4-P4-CGK4,
   wherein:
P1 to P4 each independently denote an amino acid stretch as defined in any one of claims 10 to 13,
NGK1 to NGK4 and CGK1 to CGK4 each independently denote 1 to 4 contiguous amino acids that display low beta-sheet forming potential or a propensity to disrupt beta-sheets, such as 1 to 4 contiguous amino acids selected from the group consisting of R, K, D, E, P, N, S, H, G, Q, and A, D-isomers and/or analogues thereof, and combinations thereof, preferably 1 to 4 contiguous amino acids selected from the group consisting of R, K, D, E, P, N, S, H, G, and Q, D-isomers and/or analogues thereof, and combinations thereof, more preferably 1 to 4 contiguous amino acids selected from the group consisting of R, K, D, E, and P, D-isomers and/or analogues thereof, and combinations thereof, and
Z1 to Z3 each independently denote a direct bond or preferably a linker.

Statement 15. The molecule according to any one of Statements 1 to 14, wherein the mutation or variation is a germline or somatic mutation or variation.

Statement 16. The molecule according to any one of Statements 1 to 15, wherein the mutant or variant form of the protein is causative of or associated with a disease.

Statement 17. The molecule according to Statements 16, wherein the disease is a neoplastic disease, particularly cancer.

Statement 18. The molecule according to Statement 17, wherein the protein is a proto-oncogene and the mutant or variant form of the protein is an oncogene.

Statement 19. The molecule according to any one of Statements 16 to 18 for use in medicine, particularly for use in a method of treating a disease caused by or associated with the mutant or variant form of the protein.

Statement 19′. A nucleic acid encoding the molecule according to any one of Statements 16 to 18, wherein the molecule is a polypeptide, for use in medicine, particularly for use in a method of treating a disease caused by or associated with the mutant or variant form of the protein.

Statement 20. The molecule according to Statement 17 or 18 for use in a method of treating a neoplastic disease caused by or associated with the mutant or variant form of the protein.

Statement 20′. A nucleic acid encoding the molecule according to Statement 17 or 18, wherein the molecule is a polypeptide, for use in a method of treating a neoplastic disease caused by or associated with the mutant or variant form of the protein.

Statement 21. A pharmaceutical composition comprising the molecule according to any one of Statements 1 to 18.

Statement 21′. A pharmaceutical composition comprising a nucleic acid encoding the molecule according to any one of Statements 1 to 18, wherein the molecule is a polypeptide.

Statement 22. An in vitro method for downregulating the amount or biological activity of a mutant or variant form of a protein in a cell expressing, preferably endogenously expressing, the mutant or variant form of the protein, the method comprising contacting the cell with a non-naturally occurring molecule capable of downregulating the amount or biological activity of the mutant or variant form of the protein, wherein:
a) the protein comprises a β-aggregation prone region (APR) and said APR is modified by the mutation or variation in the mutant or variant form of the protein; or
b) the mutation or variation introduces a de novo APR in the mutant or variant form of the protein not present in the protein;
and wherein the molecule is configured to specifically target the APR in the mutant or variant form of the protein; or
comprising contacting the cell with a nucleic acid encoding the molecule, wherein the molecule is a polypeptide.

Statement 23. A method for downregulating the amount or biological activity of a mutant or variant form of a protein in an organism expressing, preferably endogenously expressing, the mutant or variant form of the protein, the method comprising administering to the organism a non-naturally occurring molecule capable of downregulating the amount or biological activity of the mutant or variant form of the protein, wherein:
a) the protein comprises a β-aggregation prone region (APR) and said APR is modified by the mutation or variation in the mutant or variant form of the protein; or
b) the mutation or variation introduces a de novo APR in the mutant or variant form of the protein not present in the protein;
and wherein the molecule is configured to specifically target the APR in the mutant or variant form of the protein; or
comprising contacting the cell with a nucleic acid encoding the molecule, wherein the molecule is a polypeptide.

Statement 24. The method according to any one of Statements 22 or 23, wherein the molecule is as defined in any one of Statements 1 to 14.

Statement 25. The method according to any one of Statements 22 or 24, wherein the cell is a bacterial cell, a fungal cell, including a yeast cell or a mould cell, a protist cell, a plant cell, or an animal cell, including a non-human mammal cell or a human cell.

Statement 26. The method according to any one of Statements 23 or 24, wherein the organism is a bacterium, a fungus, including yeast or mould, a plant, or an animal.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as follows in the spirit and broad scope of the appended claims.

The herein disclosed aspects and embodiments of the invention are further supported by the following non-limiting examples.

### EXAMPLES

Materials and Methods Used in Examples 1-7

Design of RAS-Specific Aggregating Molecules (‘Pept-Ins’)

Protein sequences for RAS family member proteins were obtained from UniProt (entries: P01116 (KRAS), P01112 (HRAS) and P01111 (NRAS)) (Nucleic Acid Res. 47 (2008) 36, D190-5). Protein sequences were analyzed using the TANGO algorithm (Fernandez-Escamilla et al. 2004, supra) to identify aggregation prone regions (APRs). To this end, the following settings were used: Temperature=298K, pH=7.5, Ionic Strength=0.10 M and a cutoff on the TANGO score of 1 per residue. To assess the impact of prevalent G12 and G13 mutations on the TANGO profile, we used a sequence fragment of 19 amino acids (1-19) containing the affected APR. This sequence fragment is 100% conserved between KRAS, HRAS and NRAS, such that the outcome applies to all RAS isoforms. Mutations were introduced manually, and sequences were analyzed using the TANGO algorithm as described above.

Based on the TANGO output using both RAS wild-type and RAS G12V sequences, we generated all possible APR windows between 6 and 10 amino acids using a sliding window approach. The resulting sequence windows were cross-compared against the full human proteome and only sequences with unique exact match with RAS proteins were retained for molecule (henceforth, ‘pept-in’) design.

Peptide Synthesis and Purification

Solid Phase Peptide Synthesis

Peptide synthesis was performed on a Symphony X peptide synthesizer (Gyros Protein Technologies) at a 50 or 100 μmol scale. Rink amide low loading resin (100-200 mesh), O-(1H-6-chlorobenzotriazole-1-yl)-1,1,3,3-tetramethyluronium hexafluorophosphate (HCTU) and diethyl ether were purchased from Novabiochem/Merck. Fmoc protected amino acids (AA) and trifluoroacetic acid (TFA) were purchased from Fluorochem. N,N-Dimethylformamide (DMF), 20% piperidine in DMF solution, N,N-Diisopropylethylamine (DIPEA), triisopropylsilane (TIS) and dithiothreitol (DTT) were purchased from Sigma-Aldrich. Dichloromethane (DCM) was purchased from Acros Organics. Elongation of the desired sequences were performed by repeated cycles of Fmoc removal and coupling of amino acids (see Table 1 below for scale-depending volumes and concentrations). First, resin was swollen for 2×10 minutes in DMF. The Fmoc protecting group was next removed by exposure to a solution of 20% piperidine in DMF for 2×5 minutes using. Resin was then washed with DMF and coupling was carried out using 4 eq. AA, 4 eq. HCTU and 16 eq. DIPEA in DMF for 30 min. Resin was washed with DMF prior to next cycle. Extended Fmoc removal (2×15) minutes and double couplings (2×30 minutes) were performed from the 1^{st} AA of the second APR until the end of the desired sequence. Resin was then washed several times with DMF, DCM and then dried for 2×10 minutes. Peptide was finally cleaved from dried resin using a TFA solution containing 2.5% ultrapure water; 2.5% TIS and 2.5% DTT for 2 hours. The peptide solution was then precipitated in cold diethyl ether (35 mL for 5 mL of TFA solution) and centrifuged; liquid phase was then discarded, and peptide pellet was washed with 15 mL diethyl ether. After centrifugation, the pellet was air dried for 30 min and then dissolved in 10 mL of a water/acetonitrile solution (1:1), frozen and freeze-dried on a lyophilizer overnight to afford peptide as crude powder.

TABLE 1

| Single | | | |
|---|---|---|---|
| coupling | scale (μmol) | 50 | 100 |
| Fmoc | 20% piperidine in | 2 | 3 |
| removal | DMF (mL) | | |
| | Large DMF wash (mL) | 6 | 6 |
| | DMF wash (mL) × 4 | 2 | 4 |
| Coupling | AA (mL) | 1 | 1 |
| | HCTU solution (mL) | 1 | 1 |
| | Base (mL) | 1 | 1 |
| | DMF wash (mL) × 5 | 2 | 4 |
| | Concentration (M) | 0.2/0.19/0.8 | 0.4/0.38/1.6 |
| | AA/HCTU/Base (eq.) | 4/4/16 | 4/4/16 |
| Cleavage | scale (μmol) | 50 | 100 |
| | 2 h TFA reaction (mL) | 2.5 | 5 |
| | 1st TFA wash (mL) | 2.5 | 2.5 |
| | 2nd TFA wash (mL) | 0 | 2.5 |

Peptide Purification

Crude peptides were purified via reverse phase preparative HPLC on a Gilson system equipped with a 322 Pump, a 159 UV-vis detector and a GX281 collector using a C18 column from Phenomenex (5 μm 110 Å 250×21.2 mm, ref 006-4435-P0-AX). HPLC grade water and acetonitrile were purchased from VWR and TFA was purchased from Fluorochem. Guanidine hydrochloride (Gu) was purchased from Sigma Aldrich; dimethyl sulfoxide (DMSO) and acetic acid were purchased from Merck. Solvent A is water+0.1% TFA and solvent B is acetonitrile+0.1% TFA. Crude powder was dissolved at 20 mg/mL in DMSO, vortexed and sonicated; the solution was then diluted by a factor of 10 with Gu+10% acetic acid in water and finally filtrated on a 0.22 μm cellulose acetate filter (from Merck). Peptide solution was then purified at a 30 mL/min flow using a gradient consisting of a flat time of 7 minutes at 15% B, elution from 15% B to 45% B in 10 minutes followed by a wash of the column using 95% B for 2 minutes and an equilibration at 15% B for 6 minutes. Fractions were then analyzed by MALDI mass spectrometry. Pure fractions were pulled together in a glass vial, frozen and lyophilized over at least 2 days. Pure peptide was finally analyzed by LCMS for quality control validation using 90% purity both by UV and MS signal as threshold.

Cellular Potency Screening

Cell lines used in this application and are listed in Table 2 below:

TABLE 2

| Cell line | Supplier | Cat No |
|---|---|---|
| A-427 | ATCC | HTB-53 |
| A-549 | ATCC | CCL-185 |
| Capan-1 | CLS | 300143 |
| HCT116 | BPS Bioscience | 60520 |
| LCLC-97-TM-1 | CLS | 300409 |
| MIAPACA-2 | ATCC | CRL-1420 |
| NCI-H1299 | ATCC | CRL-5803 |
| NCI-H358 | ATCC | CRL-5807 |
| NCI-H441 | ATCC | HTB-174 |
| NCI-H727 | ATCC | CRL-5815 |
| PA-TU-8988T | DSMZ | ACC 162 |
| PANC-1 | ATCC | CRL-1469 |

DSMZ: Leibniz Institute DSMZ-German Collection of Microorganisms and Cell Cultures, Inhoffenstr. 7B, D-38124 Braunschweig Germany.
CLS: CLS Cell Lines Service, Dr. Eckener-Str. 8, D-69214 Eppelheim, Germany (www.https://clsgmbh.de/).
BPS Bioscience, 6042 Cornerstone Court West, Suite B, San Diego, CA 92121, United States (www.bpsbioscience.com).

Human tumor cell lines were obtained from ATCC (i.e. NCI-H441 (HBT-174^{TH}), NCI-H1299 (CRL-5803TM), NCI-H358 (CRL-5807TM), NCI-H727 (CRL-5815TM), A-427 (HTB-53TM), PANC-1 (CRL-1469TM), HCT-116 (CCL-247TM), and MIAPaCa-2 (CRL-1420TM)), CLS Cell Line Service GmbH (i.e. Capan-1 (300143), and LCLC-97TM1 (300409)), or Leibniz-Institut DSMZ (i.e. PA-TU-8998T (ACC 162)). Mouse embryonic fibroblasts expressing a single RAS isoform (referred to as ‘RASless MEFs’) were obtained from the Frederick National Laboratory of the National Cancer Institute, Frederick, Md., USA. All cell lines were maintained according to the provider's instructions.

Adherent Viability Assays

For the single-dose viability screen on adherent cells, 4000 cells were seeded per well in black Gclear® Cellstar® F-bottom 96-well plates (Greiner) in 100 μL full growth medium. The day after seeding, growth medium was replaced with full growth medium containing the indicated pept-in at a fixed final dose of 25 μM. Technical duplicates were included for all experimental pept-in conditions. 2 and 4 days after treatment viability was assessed using the CellTiter Blue reagent (Promega) according to the manufacturer's instructions, with the following adaptation: CellTiter Blue reagent was diluted 1 in 2 in PBS. Readout was performed on a Clariostar plate reader (BMG). Dose-response assays were performed with the following adaptations: pept-ins were tested in dose-response using a 1 in 2 dilution series with 50 μM being the highest final concentration used. Furthermore, a single viability read-out was performed 3 days after treatment using the Celltiter Glo reagent (Promega) according to the manufacturer's instructions, with the following adaptation: CellTiter Glo reagent was diluted 1 in 4 in PBS.

All test plates contained multiple normal growth and vehicle controls as well as a duplicate of a dose-response of the positive control compound SAH-SOS-1A (CAS no. 1652561-87-9).

Spheroid Viability Assays

For the single-dose viability screen on spheroid cultures, 1000 cells were seeded per well in black Ultra-Low Attachment (ULA) round-bottom 96-well plates (Corning) in 75 μL full growth medium. The day after seeding, spheroids were treated by addition of 50 μl of full growth medium containing the indicated test compounds so that the final concentration after adding was 25 μM. Technical duplicates were included for all experimental pept-in conditions 5 days after treatment viability was assessed using the CellTiter Glo 3D reagent (Promega) according to the manufacturer's instructions, with the following adaptation: 80 μL of reagent was added per well. Readout was performed on a Clariostar plate reader (BMG). For dose-response assays using RASless MEFs, cells were seeded at 1000 (G12V and G12C) or 2000 (wild-type and BRAF V600E) in Matrigel-containing medium, in order to obtain equally viable spheroids at start of treatment, 24 hrs later. Dose-response assays were performed with the following adaptation: pept-ins were tested in dose-response using a 1 in 2 dilution series with 50 μM being the highest final concentration.

All test plates contained multiple normal growth and vehicle controls as well as a duplicate of a dose-response of the positive control compound SAH-SOS-1A (Merck).

Tinctorial In Vitro Aggregation Assays

Tinctorial aggregation assays were performed using the amyloid-sensor dyes Thioflavin T (ThT) and pentameric formyl thiophene acetic acid (p-FTAA). Pept-ins were diluted from a 5 mM stock solution in 6M Urea in PBS to a final concentration of 100 μM. Measurements were performed in black half-area 96-well plates at 37° C. on a Clariostar plate reader (BMG) kinetically during 22 hours.

KRAS Aggregation Seeding Assays

Pept-ins were diluted from a 5 mM stock in 6M Urea in PBS to a final concentration of 100 μM in low-binding tubes and incubated during 20 hrs at 37° C. This solution was used either directly in subsequent seeding assays or aliquots were flash-frozen using liquid nitrogen and stored at −80° C. for later seeding assays.

For seeding assays with mature pept-in aggregates, 5 μM of the mature pept-in solution was mixed with 1 mg/ml recombinant mutant KRAS G12V in Hepes buffer containing 200 mM of arginine and glutamine. Seeding was monitored in black 384-well plates (30 μl final volume per well) using ThT as aggregation/amyloid sensor dye at 37° C. on a Clariostar plate reader (BMG).

For seeding assays with pept-in seeds, mature pept-in solutions were diluted 1 in 3 in PBS and sonicated during 5 min using cycles of 5 sec separated by a 3 sec pause. 5 μM of the sonicated pept-in solution was next mixed with 1 mg/ml recombinant mutant KRAS G12V in Hepes buffer containing 200 mM of Arginine and Glutamine. Seeding was monitored in black 384-well plates (30 μl final volume per well) using ThT as amyloid sensor dye at 37° C. on a Clariostar plate reader (BMG).

In Vitro Translation Assay

In vitro translation assays were performed using the PURExpress® In Vitro Protein Synthesis Kit (New England Biolabs) according to the manufacturer's instructions. Briefly, linear DNA fragments containing T7 promotor and terminator sequences flanking the KRAS coding sequence were generated using PCR and purified using the MinElute PCR Purification Kit (Qiagen). 250 ng of linear DNA was subsequently used for the in vitro translation reaction, which was performed for 2 hours at 37° C. with shaking (1000 rpm). Indicated biotinylated pept-ins were mixed in the translation reactions from a 5 mM stock solution in 6M Urea to a final concentration of 10 μM. Upon completion of the translation reaction, biotinylated pept-ins were captured from the reaction mix using Streptavidin coated beads (Pierce) during 90 min at room temperature. Beads were next washed with TBS containing 0.1% Tween 20 and bound proteins were finally boiled off in 1×SDS loading dye (Bio-Rad) in TBS buffer. Proteins were resolved using Any kD 15-well Mini-PROTEAN gels (Bio-Rad) during SDS-PAGE and probed for KRAS after Western blotting using a mouse monoclonal KRAS-specific antibody (SC-30, Santa Cruz Biotechnology), which was detected with an HRP-coupled anti-mouse secondary antibody using chemiluminescence on a Bio-Rad Chemidoc MP imaging instrument.

Co-Immunoprecipitation Assays

Cellular co-immunoprecipitation assays were performed using either KRAS wild-type or mutant G12V expressing RASless MEFs (see elsewhere) or human NCI-H441 lung adenocarcinoma tumor cells and N-terminally biotinylated pept-ins. Cells were seeded at a density of 300,000 cells in a clear 6-well plate (Cellstar, Greiner). One day after seeding, cells were treated with indicated pept-ins at a final concentration of 25 μM and incubated for 20 hours. Next, cells were lysed with NP-40 lysis buffer (150 mM NaCl, 50 mM Tris HCl pH8, 1% IGEPAL(NP40), 1×Halt phosphatase/protease inhibitors (Thermo), 1 U/μl Universal Nuclease (Pierce)) and biotinylated pept-ins were captured with streptavidin-coated magnetic beads (Pierce) during 1 hours at room temperature. Beads were washed with NP40 lysis buffer at least 3 times, after which bound proteins were boiled off in 1×SDS loading dye (Bio-Rad) in NP40 lysis buffer. Proteins were resolved using Any kD 15-well Mini-PROTEAN gels (Bio-Rad) during SDS-PAGE and probed for KRAS after Western blotting using a rabbit polyclonal KRAS-specific antibody (12063-1-AP, Proteintech).

Flow Cytometry

NCI-H441 cells were seeded in a 12-well plate at a density of 175k cells/well. Next day, cells were treated with vehicle or 12.5 μM of the RAS-targeting pept-ins or the negative control pept-in. After 6, 16 and 24 hours of treatment, cells were washed with PBS and detached using TrypLE Express (Thermo Fisher). Washed cells were next stained using Sytox Blue (Thermo Fisher) and Amytracker Red (Ebba Biotech AB), before analyzing them on a Gallios flow cytometer (Beckman Coulter).

Cellular Fluorescent Imaging

Fluorescent cellular imaging was performed using HeLa cells that were transduced with lentiviral particles carrying a construct expressing KRAS G12V labeled N-terminally with mCherry. Cells were seeded in a black Gclear® Cellstar® F-bottom 96-well plates (Greiner) in 100 μL full growth medium. One day later, cells were treated with indicated FITC-labeled pept-ins in normal growth medium during 20 min after which the pept-in solution was washed off and replaced with normal growth medium again and incubated for an additional 2 hours. Next, cells were fixed, washed and counterstained with the nuclear dye NucBlue™ (containing Hoechst 33342). Images were captured on a Leica confocal microscope.

In Vivo SW620 Xenograft Model

Female NCr nu/nu mice (8 to 12 weeks) were inoculated with 1×10⁶ SW620 tumor cells in 50% Matrigel subcutaneously in the hind flank. The cell Injection Volume was 0.1 mL/mouse. When tumors reached an average size of 100-150 mm³ a pair match was performed, and treatment started. Group sizes were N=6 for the non-treated group, N=5 for the vehicle groups and N=8 for the pept-in and positive control groups. Tumor growth was monitored by caliper measurement twice per week. Model response was monitored by Irinotecan dosed once per week at 100 mg/kg intraperitoneally for 3 weeks.

### Example 1: Design of RAS-Specific Aggregating Molecules (‘Pept-Ins’)

We used the statistical thermodynamics algorithm TANGO to identify aggregation prone regions (APRs) in the primary amino acid sequence of human RAS family proteins (HRAS, NRAS and KRAS). This analysis showed that all 3 RAS family members have an identical TANGO profile with each of them carrying 5 APRs of at least 5 amino acids in length, of which 2 APRs have a TANGO score of at least 20% (Table 3). The start position (‘Start’) of a given APR as indicated in Table 3, corresponds to the position, in the RAS sequence, of the first N-terminal gatekeeper preceding the respective aggregation prone region per se, whereas elsewhere in this specification the start position of the APR may be given without the N-terminal gatekeeper. Hence, for example, the N-terminal most APR of RAS is stated in Table 3 to start at the M gatekeeper at position 1 of RAS, whereas this APR may be stated to start with T at position 2 elsewhere in this specification. Further in Table 3, ‘N-GKs’ denotes the native gatekeeper residues N-terminally adjacent to the predicted APR in RAS, ‘C-GKs’ denotes the native gatekeeper residues C-terminally adjacent to the predicted APR in RAS, ‘APR seq’ denotes the APR sequence, ‘Score’ means TANGO score in %, and ‘Length’ denotes the APR length (aa) excluding any gatekeepers.

TABLE 3
TANGO analysis of RAS family proteins.

| Protein | Start | N-GKs | APR seq | C-GKs | Score | Length |
|---|---|---|---|---|---|---|
| HRAS | 1 | M | TEYKLVVVGAG | GVG | 20.2368 | 11 |
| | | | SEQ ID NO: 2 | | | |
| HRAS | 17 | GKS | ALTIQLI | QNH | 9.34057 | 7 |
| | | | SEQ ID NO: 40 | | | |
| HRAS | 76 | TGE | GFLCVFAIN | NTK | 68.1289 | 9 |
| | | | SEQ ID NO: 41 | | | |
| HRAS | 110 | DVP | MVLVG | NKC | 3.08482 | 5 |
| | | | SEQ ID NO: 42 | | | |
| HRAS | 154 | VED | AFYTLV | REI | 56.7861 | 6 |
| | | | SEQ ID NO: 43 | | | |
| KRAS | 1 | M | TEYKLVVVGAG | GVG | 20.5293 | 11 |
| | | | SEQ ID NO: 2 | | | |
| KRAS | 17 | GKS | ALTIQLI | QNH | 9.53801 | 7 |
| | | | SEQ ID NO: 40 | | | |
| KRAS | 76 | TGE | GFLCVFAIN | NTK | 68.2723 | 9 |
| | | | SEQ ID NO: 41 | | | |
| KRAS | 110 | DVP | MVLVG | NKC | 3.1616 | 5 |
| | | | SEQ ID NO: 42 | | | |
| KRAS | 154 | VED | AFYTLV | REI | 56.8076 | 6 |
| | | | SEQ ID NO: 43 | | | |
| NRAS | 1 | M | TEYKLVVVGAG | GVG | 20.1731 | 11 |
| | | | SEQ ID NO: 2 | | | |
| NRAS | 17 | GKS | ALTIQLI | QNH | 9.29791 | 7 |
| | | | SEQ ID NO: 40 | | | |
| NRAS | 76 | TGE | GFLCVFAIN | NTK | 67.981 | 9 |
| | | | SEQ ID NO: 41 | | | |
| NRAS | 110 | DVP | MVLVG | NKC | 3.07989 | 5 |
| | | | SEQ ID NO: 42 | | | |
| NRAS | 154 | VED | AFYTLV | REI | 56.4851 | 6 |
| | | | SEQ ID NO: 43 | | | |

Activating mutations in RAS family members are a common and often early event in human cancers and it has been reported that up to one-third of all human tumors carry missense mutations in one of the RAS family members. Greater than 99% of these mutations occur at so-called hotspot mutation sites which are again shared among the RAS family members and are located at codons 12, 13 and 61. Interestingly, codon 12 is located at the C-terminus of an APR, and codon 13 is located immediately adjacent to the C-terminus of an APR, and a missense mutation at one of these positions might therefore alter the aggregation propensity but also the sequence selectivity of the aggregation process (Table 3). To study the former, we analyzed how a set of prevalent mutations (>1% over all KRAS mutant cancers) at codons 12 or 13 alters the TANGO output of the sequence (Table 4). In Table 4, ‘Score’ means TANGO score in %, ‘Length’ denotes the APR length (aa) excluding any gatekeepers, and ‘Frequency’ denotes frequency of the particular G12 or G13 mutation in all KRAS mutant cancers in % based on COSMIC database.

TABLE 4
Impact of common G12 or G13 position
mutations on TANGO analysis.

| Mutation | | Score | | |
|---|---|---|---|---|
| at G12 | APR sequence | (%) | Length | Frequency |
| WT | TEYKLVVVGAG | 20.0763 | 11 | 1 |
| | SEQ ID NO: 2 | | | |
| G12V | TEYKLVVVGAVGVG | 44.8509 | 14 | 23 |
| | SEQ ID NO: 3 | | | |
| G12D | TEYKLVVVGA | 40.8683 | 10 | 34 |
| | SEQ ID NO: 44 | | | |
| G12C | TEYKLVVVGACGV | 19.5246 | 13 | 12 |
| | SEQ ID NO: 4 | | | |
| G12A | TEYKLVVVGAAGV | 23.9605 | 13 | 5 |
| | SEQ ID NO: 5 | | | |
| G12S | TEYKLVVVGASG | 18.8902 | 12 | 5 |
| | SEQ ID NO: 6 | | | |
| G12R | TEYKLVVVGA | 12.8417 | 10 | 3 |
| | SEQ ID NO: 44 | | | |
| G13D | TEYKLVVVGAG | 34.9655 | 11 | |
| | SEQ ID NO: 2 | | | |
| G13C | TEYKLVVVGAGCV | 18.3813 | 13 | |
| | SEQ ID NO: 7 | | | |
| G13V | TEYKLVVVGAGVVG | 40.8491 | 14 | |
| | SEQ ID NO: 8 | | | |
| G13R | TEYKLVVVGA | 17.3727 | 10 | |
| | SEQ ID NO: 44 | | | |
| G13S | TEYKLVVVGAGS | 18.6667 | 12 | |
| | SEQ ID NO: 9 | | | |

The most prevalent mutation at position G12 is G12D. This mutation introduces a negatively charged aspartate which TANGO identifies as a gate-keeper residue, resulting in a slightly shorter APR with an increased TANGO score. However, the impact of the second most prevalent mutation, G12V, on the APR is most profound as it increases both the length as well as the TANGO score of the APR sequence. Other prevalent G12 mutations either shorten or lengthen the APR sequence but do not alter the TANGO score significantly. G13D mutation is also very prevalent and increases the aggregation propensity of the APR without altering its sequence. Hence, it is possible that a pept-in having a stretch corresponding to the wild-type APR may display a preference for downregulating G13D RAS compared to wild-type RAS. The impact of the G13V on the APR is also very profound as it increases both the length as well as the TANGO score of the APR sequence.

Based on these data, we selected the RAS WT and G12V APRs for the design of RAS WT or G12V-selective pept-ins, as embodiments illustrating the feasibility of specifically targeting G12 or G13 mutant human RAS using the interferor technology.

To this end we generated all possible 6 to 10-mers (in the present experiments, the length limit of amino acids was informed by the length capacity of solid phase synthesis) based on the sequence of the APRs using a sliding window approach. Next, the resulting ‘APR windows’ were aligned against the full human proteome to exclude sequences that had exact matches in other proteins than the RAS family members to limit off-target activity of pept-ins containing these sequences. This filtering step resulted in 38 APR windows that were taken further in our pept-in design. For the design we employed the previously devised tandem repeat configuration (see WO2012/123419A1), in which the APR windows are repeated once and are separated by a linker. For the design of the initial screening library, we included variants with both GS and PP linkers. Furthermore, to increase the colloidal stability of these aggregating sequences, gatekeeper residues were introduced flanking each repeat of the APR window in the pept-in. Two positively charged (Arginine (R) and Lysine (K)) and one negatively charged (Aspartate (D)) amino acids were selected and introduced in the screening library. An overview of the resulting pept-in templates with different gate keeper residues and linkers is given in Table 5. The K-APR-KGSK-APR-K template was applied to all APR windows, while the other templates were applied to all APR windows up to 8 amino acids in length.

TABLE 5
Overview of pept-in design
templates for screening.

| Gate- | | |
|---|---|---|
| keeper | | |
| residue | Linker | Pept-in layout |
| K | GS | K-APR-KGSK-APR-K |
| R | GS | R-APR-RGSR-APR-R |
| K | PP | K-APR-KPPK-APR-K |
| D | PP | D-APR-DPPD-APR-D |
| KK | PP | KK-APR-KPPK-APR-KK |

All pept-ins designed were generated using solid phase synthesis, however, for a few sequences synthesis or purification failed to meet the quality standards (purity>95%) and were therefore excluded from further analysis. Pept-ins for which synthesis and purification was successful were dissolved in 6M Urea to a 5 mM stock and tested for their biological activity.

### Example 2: Activity Screening of RAS-Targeting Pept-Ins

To assess pept-in activity on the viability of RAS-mutant tumor cells, we used adherent NCI-H441 lung adenocarcinoma cells which harbor a G12V mutation in KRAS. To verify that this cell line was indeed dependent on KRAS for its growth, we used SAH-SOS-1A as a positive control. SAH-SOS-1A is a peptidic compound whose design is based on a stabilized helix from son of sevenless 1, the canonical guanine exchange factor for KRAS (Leshchiner et al. Proc Natl Acad Sci USA. 2015, vol. 112(6), 1761-6). Treatment of NCI-H441 cells with SAH-SOS-1A resulted in a dose-dependent drop in viability with an IC₅₀ of ˜-15 μM after 4 days exposure, which was consistent with reported values for other cell lines and established the KRAS-dependence for the NCI-H441 cell line. We also tested Urea tolerance of NCI-H441 cells and found that there was no significant effect on viability up to 60 mM of Urea after 4 days of exposure.

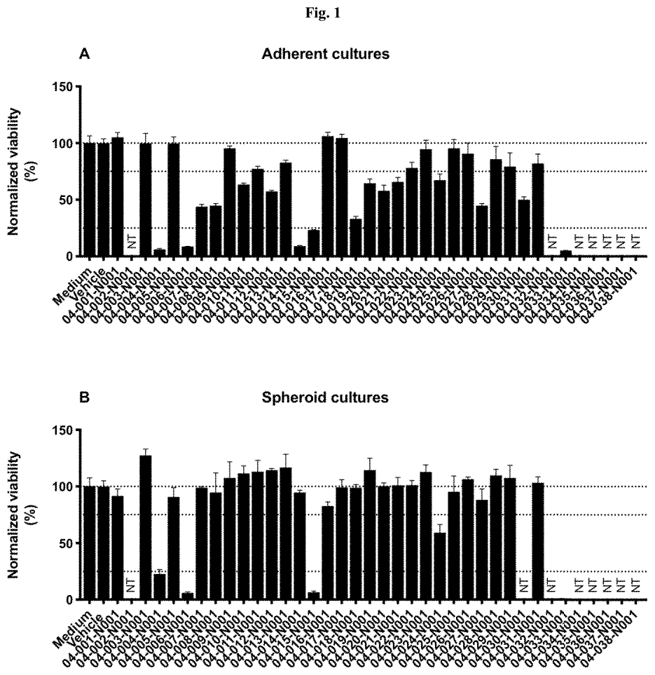

Pept-ins were screened at a single dose of 25 μM (corresponds to final concentration of 30 mM Urea) and viability was measured after 2 and 4 days of exposure using the CellTiter Blue reagent. After 4 days of exposure over half of all K-APR-KGSK-APR-K pept-ins tested (˜-52%) induced a reduction of at least 25% in viability as compared to vehicle treated cells (30 mM Urea; FIG. 1A). Hit rates and potencies for the other templates tested were considerably lower. To select potent hits for further characterization, we selected all pept-ins that showed at least 75% decrease in viability after 4 days of exposure. This cut-off resulted in selection of 5 pept-ins, all with the K-APR-KGSK-APR-K template: 04-004-N001, 04-006-N001, 04-014-N001, 04-015-N001 and 04-033-N001. One of these pept-ins (04-004-N001) harbours an APR window sequence derived from another APR of RAS, that is thus present in both G12 mutant and wild-type RAS, while the other four pept-ins (04-006-N001, 04-014-N001, 04-015-N001 and 04-033-N001) harbour an APR window sequence that is derived from and contains a G12V mutant site. Furthermore, we selected one biologically non-active peptide (04-016-N001) to be used as negative control in later assays. This pept-in carries a 7-mer APR window that was designed to target RAS G12V but failed to alter viability of the NCI-H441 cells.

The sequences of the aforementioned pept-ins are shown in Table 6.

| | | Normalized |
|---|---|---|
| | | viability |
| | | NCI-H441 |
| | | 4 days of |
| | | exposure |
| Pept-in | | to 25 μM |
| code | Sequence | (%) |
| 04-004-N001 | Ac-KLSVFAIKGSKLS | 6.1 |
| | VFAIK-NH2 | |
| 04-006-N001 | Ac-KVVVGAVKGSKVV | 8.7 |
| | VGAVK-NH2 | |
| 04-014-N001 | Ac-KLVVVGAVKGSKL | 9.1 |
| | VVVGAVK-NH2 | |
| 04-015-N001 | Ac-KVVVGAVGKGSKV | 23.3 |
| | VVVGAVGK-NH2 | |
| 04-033-N001 | Ac-KVVVGAVGVGKGS | 5.2 |
| | KVVVGAVGVGK-NH2 | |

The amino acid sequence of pept-in 04-004-N001 as shown in Table 6 is assigned SEQ ID NO: 69, while the amino acid sequences of pept-ins 04-006-N001, 04-014-N001, 04-015-N001 and 04-033-N001 are represented as SEQ ID NO: 17, SEQ ID NO: 18, SEQ ID NO: 19, and SEQ ID NO: 20, respectively, as also set forth elsewhere in this specification. ‘Ac’ in Table 6 denotes N-terminus acetylation, and ‘NH2’ in Table 6 denotes C-terminus amidation.

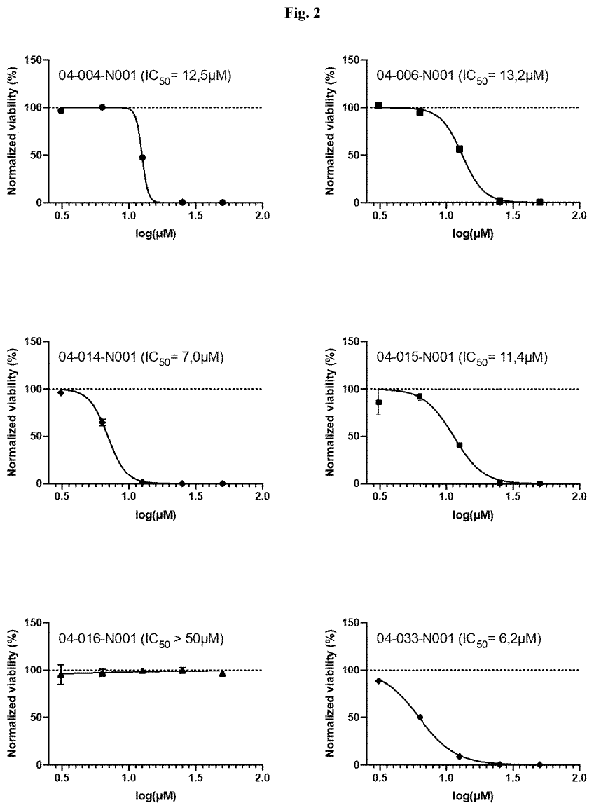

These 6 pept-ins were resynthesized and purified to test their potency in reducing viability of adherently growing (‘2D viability assay’) NCI-H441 cells in dose-response. To this end, pept-ins were tested in a five-point dose-response using a one-in-two dilution series starting from 50 μM as highest dose on adherently growing NCI-H441 cells. Viability was assessed three days after of exposure to the test compounds using the CellTiter Glo viability assay. This analysis showed that the 5 active compounds all showed IC₅₀s around 10 μM (FIG. 2).

As previous reports have shown that adherent growth of KRAS mutant cells lines might attenuate their sensitivity to KRAS inhibition or knockdown (Fujita-Sato et al. Cancer Res. 2015, vol. 75, 2851-62; Patricelli et al. Cancer Discov. 2016, vol. 6, 316-29; Vartanian et al. J Biol Chem. 2013, vol. 288, 2403-13), we complemented the screen on adherently growing NCI-H441 cells with a screen on suspension spheroid cultures of the same cell line. To this end, NCI-H441 cells were seeded in ultra-low adherent round bottom plates allowing formation of spheroids. As for the adherent screen, we adopted a single-dose approach using 25 μM of each test pept-in. Viability of the spheroid cultures was determined after 5 days of exposure using the CellTiter Glo 3D reagent from Promega. Hit rates using this approach were considerably lower as compared to the adherent screen described above (FIG. 1B). Indeed, while in the adherent setting over half of all K-APR-KGSK-APR-K pept-ins tested induced a reduction of at least 25% in viability as compared to vehicle treated cells, in the spheroid setting only 17% of this set of pept-ins reduced viability with more than 25%. Furthermore, hit rates and potencies for the other templates tested were also lower. Of note, applying the same selection criterion for potent hits here as for the adherent screen, i.e. selecting pept-ins that showed at least 75% decrease in viability after 5 days of exposure, resulted in the selection of same pept-ins as in the adherent screen, with the exception of 04-014-N001, which did not display activity in the spheroid setting.

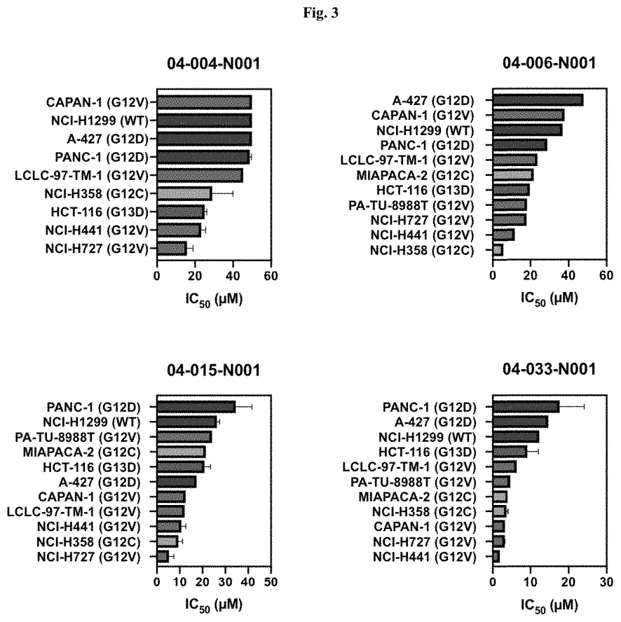

The suspension spheroid approach was used next to assess efficacy of the four active pept-ins on a larger set of KRAS mutant and wild-type tumor cells lines. Waterfall plots for each pept-in showing the median IC₅₀ for these cell lines are shown in FIG. 3.

The suspension spheroid approach was used next to assess efficacy of various versions of the 04-004, 004-006, 04-015 and 04-033 pept-ins containing alternative gatekeeper and/or linker parts, in NCI-H441 lung adenocarcinoma cells. IC50 on cell viability were determined using the CellTiter Glo 3D assay (Promega) after 5 days of exposure to a dose-response of each pept-in. The pept-ins and the respective IC50 values are listed in Table 7 below (‘Ac’ denotes N-terminus acetylation; ‘NH2’ denotes C-terminus amidation; ‘[Dap]’ denotes diaminopimelic acid; ‘[Cit]’ denotes citrulline; L-amino acids are represented using capital letter coding; D-amino acids are represented by small letter coding):

TABLE 7
IC50 on cell viability for various
pept-ins as disclosed herein

| | APR/ | Full sequence/ | IC50 |
|---|---|---|---|
| Pept-ins | SEQ ID NO | SEQ ID NO | (μM) |
| 04-004-N021 | LSVFAI | Ac-kLSVFAIKGSKLSVFAIk-NH2 | 34.6 |
| | 71 | 75 | |
| 04-006-N021 | VVVGAV | Ac-kVVVGAVKGSKVVVGAVk-NH2 | 49.9 |
| | 72 | 76 | |
| 04-015-N009 | VVVGAVG | Ac-kVVVGAVGKGSKVVVGAVGk-NH2 | 16.4 |
| | 73 | 77 | |
| 04-033-N021 | VVVGAVGVG | Ac-kVVVGAVGVGKGSKVVVGAVGVGk-NH2 | 8.4 |
| | 74 | 78 | |
| 04-004-N022 | LSVFAI | Ac-[Dap]LSVFAIKGSKLSVFAI[Dap]-NH2 | 19.9 |
| | 71 | 79 | |
| 04-006-N022 | VVVGAV | Ac-[Dap]VVVGAVKGSKVVVGAV[Dap]-NH2 | 19.1 |
| | 72 | 80 | |
| 04-015-N012 | VVVGAVG | Ac-[Dap]VVVGAVGKGSKVVVGAVG[Dap]- | 25.6 |
| | 73 | NH2 | |
| | | 81 | |
| 04-033-N022 | VVVGAVGVG | Ac-[Dap]VVVGAVGVGKGSKVVVGAVGVG | 5.0 |
| | 74 | [Dap]-NH2 | |
| | | 82 | |
| 04-006-N074 | VVVGAV | Ac-[Cit]VVVGAVKGSKVVVGAVK-NH2 | 25.3 |
| | 72 | 83 | |
| 04-006-N075 | VVVGAV | Ac-KVVVGAV[Cit]GSKVVVGAVK | 6.5 |
| | 72 | 84 | |
| 04-006-N044 | VVVGAV | Ac-AVVVGAVKGSKVVVGAVK-NH2 | 5.3 |
| | 72 | 85 | |
| 04-006-N050 | VVVGAV | Ac-KVVVGAVAGSKVVVGAVK-NH2 | 5.7 |
| | 72 | 86 | |
| 04-006-N053 | VVVGAV | Ac-KVVVGAVKGSAVVVGAVK-NH2 | 21.6 |
| | 72 | 87 | |
| 04-006-N059 | VVVGAV | Ac-KVVVGAVKGSKVVVGAVA-NH2 | 15.7 |
| | 72 | 88 | |
| 04-006-N060 | VVVGAV | Ac-AVVVGAVKGSAVVVGAVK-NH2 | 31.7 |
| | 72 | 89 | |
| 04-006-N066 | VVVGAV | Ac-KVVVGAVAGSKVVVGAVA-NH2 | 25.3 |
| | 72 | 90 | |
| 04-006-N082 | VVVGAV | Ac-AVVVGAVAGSKVVVGAVK-NH2 | 18.2 |
| | 72 | 91 | |
| 04-006-N051 | VVVGAV | Ac-KVVVGAVKASKVVVGAVK-NH2 | 10.4 |
| | 72 | 92 | |
| 04-006-N052 | VVVGAV | Ac-KVVVGAVKGAKVVVGAVK-NH2 | 30.6 |
| | 72 | 93 | |
| 04-015-N063 | VVVGAVG | Ac-KVVVGAVGKGFKVVVGAVGK-NH2 | 38.7 |
| | 72 | 94 | |
| 04-015-N064 | VVVGAVG | Ac-KVVVGAVGKFFKVVVGAVGK-NH2 | 48.9 |
| | 72 | 95 | |
| 04-004-N016 | LSVFAI | Ac-KLSVFAIKKLSVFAIK-NH2 | 45.9 |
| | 71 | 96 | |
| 04-033-N007 | VVVGAVGVG | Ac-KVVVGAVGVGKKVVVGAVGVGK-NH2 | 13.1 |
| | 74 | 97 | |
| 04-004-N030 | lsvfai | Ac-klsvfaikGsklsvfaik-NH2 | 21.4 |
| | | 98 | |
| 04-006-N030 | vvvGav | Ac-kvvvGavkGskvvvGavk-NH2 | 15.7 |
| | | 99 | |
| 04-033-N030 | vvvGavGvG | Ac-kvvvGavGvGkGskvvvGavGvGk-NH2 | 5.5 |
| | | 100 | |

Table 7 shows that persuasive IC50 values on cell viability have been demonstrated by molecules which exemplify various embodiments of the pept-ins as disclosed herein, such as, peptin-ins containing one or more D-lysine (‘k’), diaminopimelic acid (‘[Dap]’), citrulline (‘[Cit]’), or L-alanine (‘A’) within one or more of their gatekeeper stretches; one or more L-alanine (‘A’) or L-phenylalanine (‘F’), or one or more D-serine (‘s’) within their linker moiety or even not comprising any linker moiety; and/or composed entirely of D-amino acids and glycine. These pept-ins demonstrate the structural flexibility of the present approach focused on targeting the aggregation-prone stretches within proteins.

### Example 3: RAS-Targeting Pept-Ins are Aggregation-Prone and Seed Aggregation of RAS Through Direct Interaction In Vitro

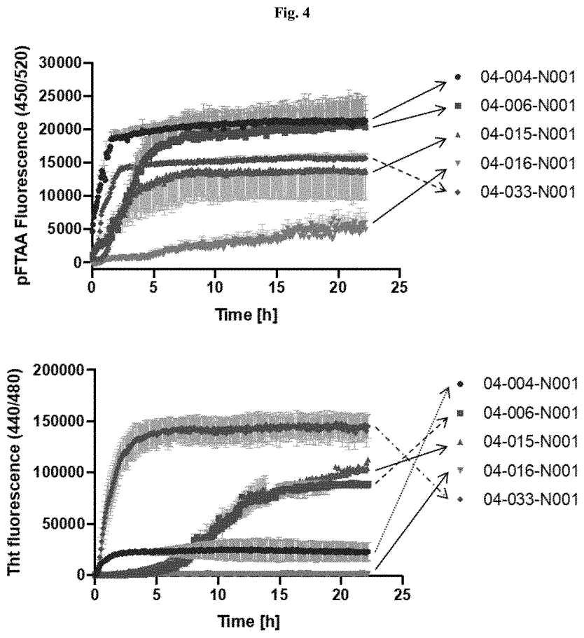

To study the aggregation behaviour of the RAS-targeting pept-ins, we performed kinetic tinctorial assays using the amyloid aggregate sensor dyes Thioflavin T (ThT) and pentameric formyl thiophene acetic acid (p-FTAA). All four representative biologically active pept-ins showed clear amyloid-aggregation kinetics with both dyes, while the inactive control showed no significant ThT signal and only a slight increase in p-FTAA signal over time (FIG. 4).

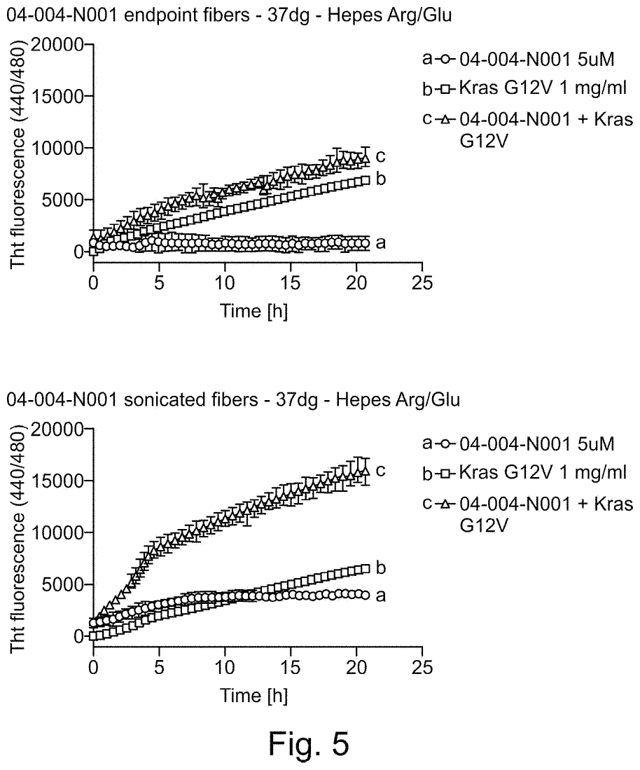

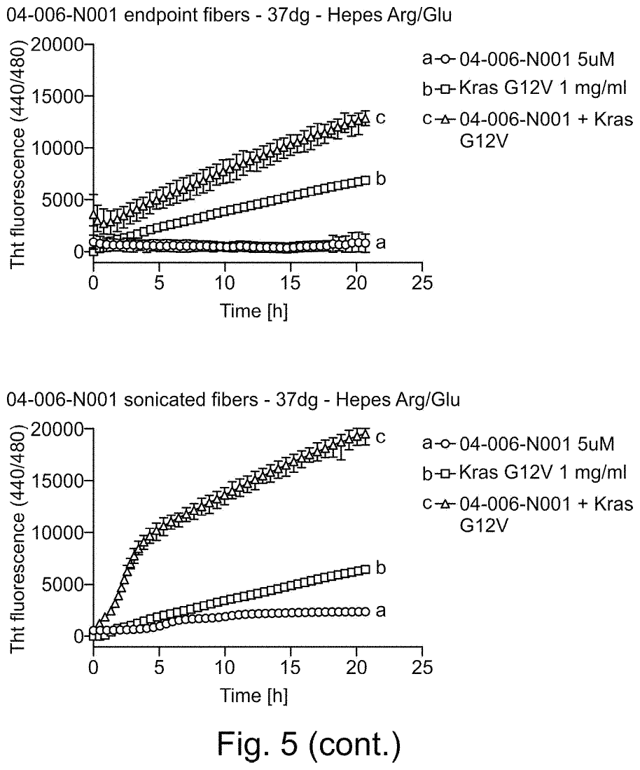

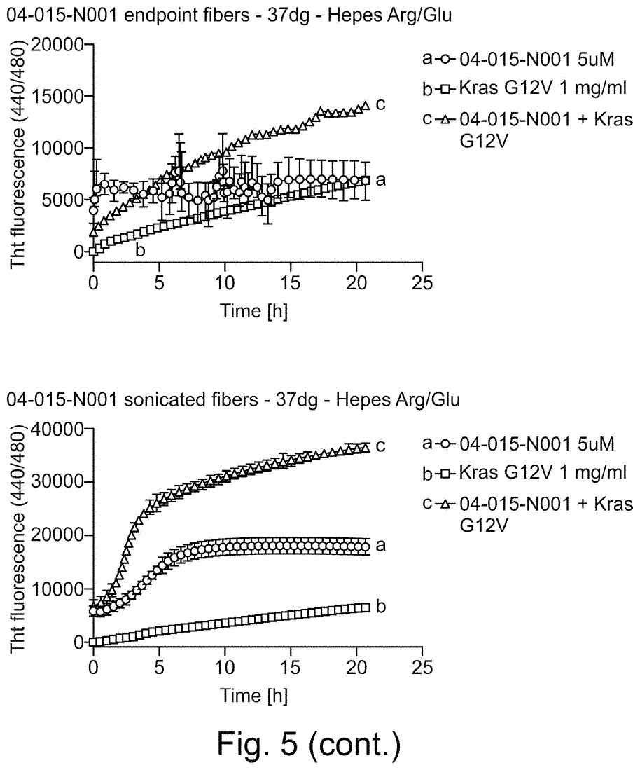

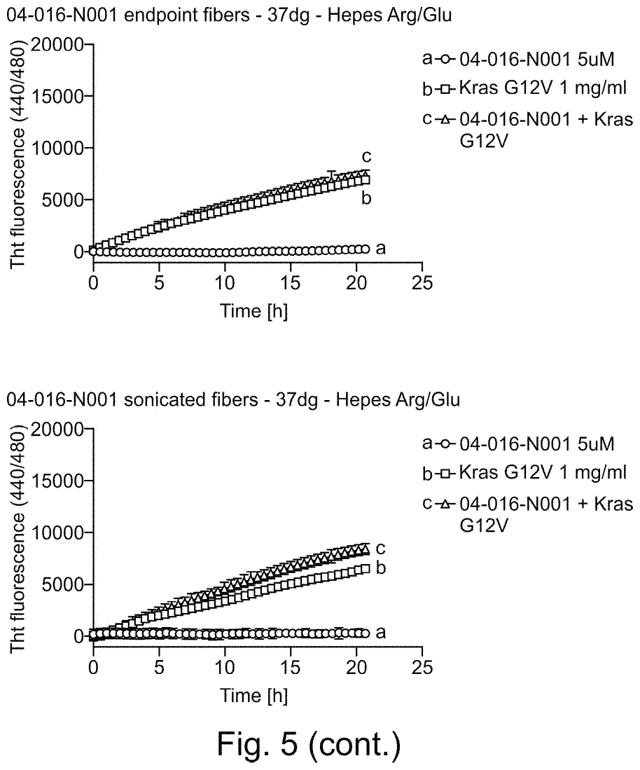

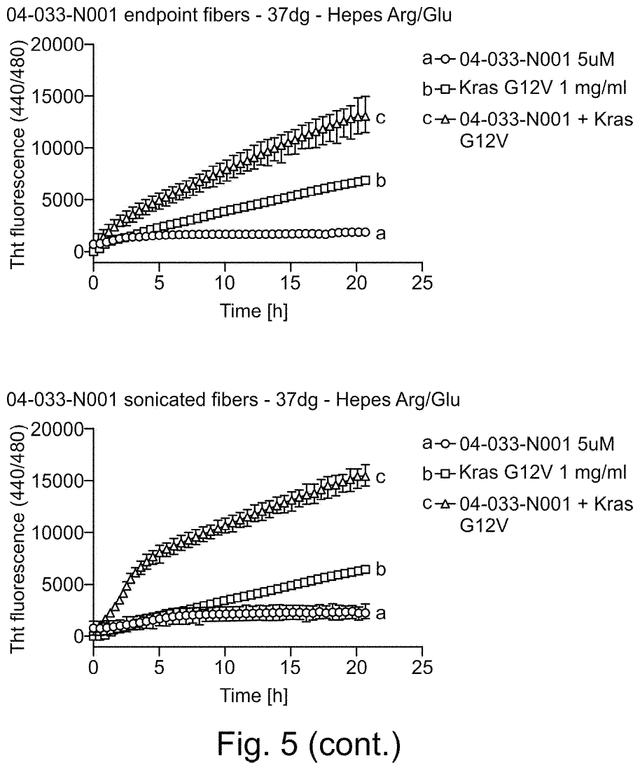

To show that the illustrative biologically active pept-ins are indeed able to target and seed the aggregation of their target protein, KRAS G12V, we performed seeding experiments with end-stage aggregates or sonicated seeds of the different KRAS-targeting pept-ins. To this end, pept-ins were allowed to aggregate in the same timeframe as for the tinctorial kinetic assays. End-stage samples were then mixed with recombinantly produced KRAS G12V and aggregation was monitored kinetically using ThT. This approach revealed only minor seeding capacity of these end-stage pept-in aggregates on KRAS G12V. However, upon disruption of the mature aggregates through sonication, potent seeds are formed which efficiently induce aggregation of KRAS G12V (FIG. 5).

To show that the RAS-targeting pept-ins interact directly with the RAS protein we setup an in vitro translation assay. Indeed, as the available structural data show that the RAS APRs may not be exposed in the native fold, we hypothesize that initial interaction of pept-ins with their target occurs at the ribosome while the protein is being translated and briefly exposes these APRs. To mimic this in vitro, we devised an in vitro translation setup producing either wild-type or mutant KRAS (G12V, G12C, G12D or G13D) in the presence of biotinylated RAS-targeting pept-ins. This allowed us to perform a streptavidin pull-down to capture the biotinylated pept-ins from the translation reaction and perform SDS-PAGE and Western blotting to probe the pulled-down fraction for the presence of KRAS. The biotinylated version of pept-in 04-004-N001, i.e. 04-004-N011, which harbours an APR window sequence derived from a wild-type APR, is predicted to target all RAS proteins independently from their mutation status. While efficient pull-down with 04-004-N011 was indeed observed for KRAS wild-type, G12V and G12C, binding to the G12D and G13D mutants appeared to be less efficient. Using the biotinylated versions of the biologically active pept-ins harbouring an APR window containing the G12V mutant site (04-006-N007, 04-015-N026 and 04-033-N003), however, notable pull-down was only observed for the G12V mutant KRAS and, in the case of 04-015-N026, for the G12C mutant KRAS (FIG. 6).

Together, these data show that these illustrative RAS-targeting pept-ins are able to directly interact with and seed the aggregation of RAS proteins containing an exact match for the APR windows present in the pept-ins.

### Example 4: Mutant-Selective Cellular Efficacy in the RASless MEF System

RAS mutant-selectivity on cellular efficacy was assessed using the isogenic RASless mouse embryonic fibroblast (MEF) panel. These MEFs are derived from NRAS- and HRAS-null mice in which the KRAS gene has been floxed as well (removal by ER-Cre). Proliferation is dependent on the expression of either the endogenous KRAS gene or—if it has been removed through tamoxifen treatment—on an expressed transgene. The panel assessed included the common clinical KRAS variants expressed as transgene (WT, G12V and G12C) and an additional cell line dependent on the expression of BRAF V600E for proliferation. The latter should be refractory to KRAS targeting agents as they do not express any of the RAS isoforms and proliferation of these cells is exclusively dependent on mutant BRAF, which is downstream of RAS.

Efficacy of RAS-targeting pept-ins on MEFs growing as spheroids was assessed after 5 days of exposure. As the targeting moiety of 04-004-N001 is an APR-window derived from a wild-type RAS sequence, it is predicted to target all RAS-dependent growth, independent from mutation status. Surprisingly, however, notable increased efficacy of 04-004-N001 was observed for the MEFs expressing KRAS G12V as compared to the KRAS WT and G12C expressing MEFs, which responded similarly as the BRAF V600E expressing RASless MEFs.

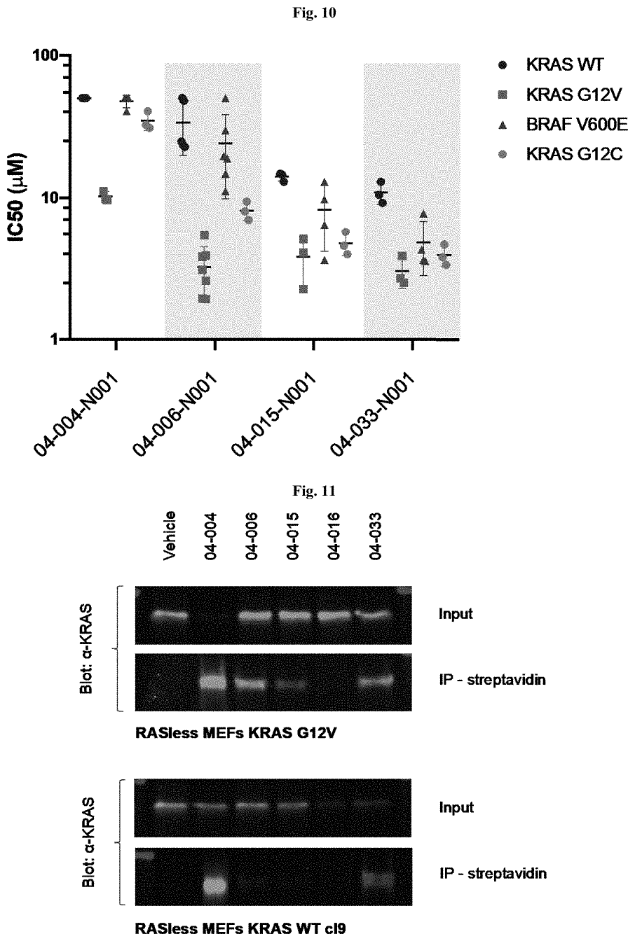

For the G12V-targeting RAS pept-ins the highest efficacy was observed when assessing the G12V-expressing RASless MEFs, indicating that mutant-selective binding at least in part drives, and may be a major contributor to, the selectivity for mutant RAS displayed by these pept-ins. The data is shown in FIG. 10.

### Example 5: RAS-Targeting Pept-Ins Interact with KRAS

To assess whether the RAS-targeting pept-ins are also able to interact with the (mutant) KRAS protein in cells, we setup a co-immunoprecipitation assay.

First, we used the KRAS wild-type and mutant G12V-expressing RASless MEFs to assess whether (i) the RAS-targeting pept-ins bind the KRAS protein in a cellular environment and (ii) whether any binding shows similar G12V mutant-selectivity as observed in the in vitro translation assay described in Example 4. To this end, relevant MEF cells were treated with 25 μM biotinylated pept-ins overnight (16 hours). Next, cells were lysed, and pept-ins were immunoprecipitated from the lysates using streptavidin-coated beads. Precipitated fractions were next resolved using SDS PAGE and probed for the presence of KRAS protein using Western blot. Results show that the 04-004-derived biotinylated pept-in appeared to precipitate both wild-type and mutant G12V KRAS well after 16-hour treatment of the respective RASless MEF cells. Treatment and precipitation with the biotinylated versions of the G12V-selective pept-ins, however, showed preferential binding to the G12V mutant KRAS protein (FIG. 11).

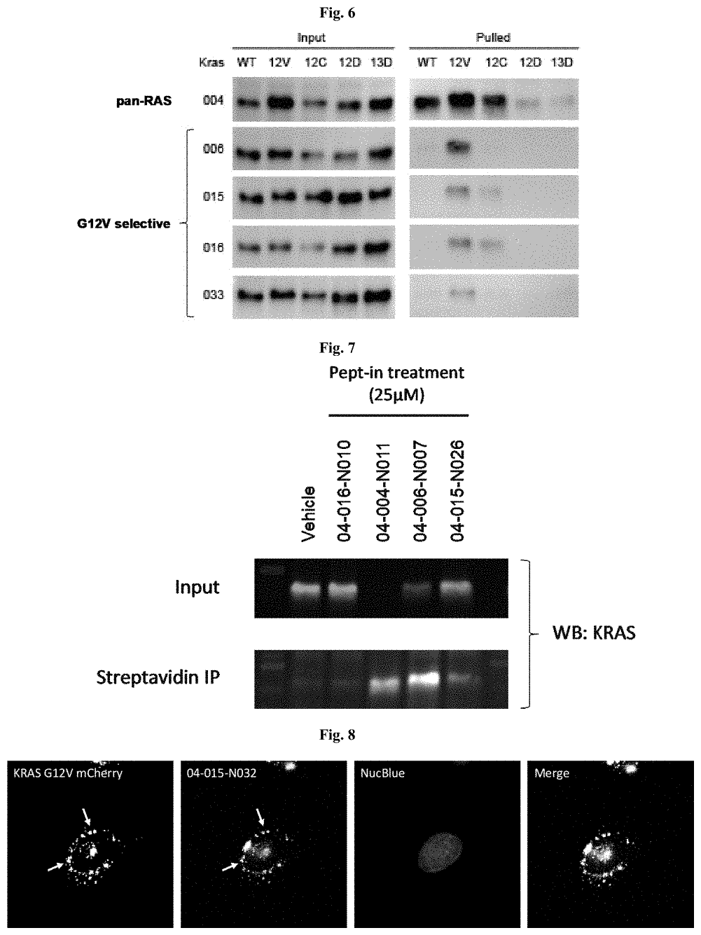

Next, we assessed whether the RAS-targeting pept-ins showed binding to KRAS after exposure to human tumor cells. To this end, the KRAS G12V mutant NCI-H441 lung adenocarcinoma cells were treated with 25 μM biotinylated pept-ins overnight (16 hrs). Next, cells were lysed, and pept-ins were immunoprecipitated from the lysates using streptavidin-coated beads. Precipitated fractions were next resolved using SDS PAGE and probed for the presence of KRAS protein using Western blot. While this approach yielded no detectable KRAS protein in the precipitated fractions from vehicle or negative control peptide-treated conditions, KRAS protein was readily detected in the precipitated fractions from NCI-H441 cells treated with the biologically active pept-ins (FIG. 7).

To complement the co-immunoprecipitation approach, we also used a cellular imaging approach to show target engagement. To this end, we generated a HeLa cell line overexpressing mCherry-tagged KRAS G12V and FITC-labelled versions of the RAS-targeting pept-ins. Treatment of these HeLa cells showed that the FITC-labelled versions of all biologically active RAS-targeting pept-ins are readily taken up by cells, while uptake of the FITC-labelled version of the negative control pept-in 04-016-N001 was not detectable, hence explaining the lack of biological activity. Furthermore, this analysis showed that rapidly after entering the cells, the RAS-targeting FITC-labelled version of pept-in 04-015-N001 (04-015-N032) associates with mCherry-labelled KRAS as revealed by the occurrence of inclusion-like perinuclear structures that are positive for both FITC as well as mCherry 75 min after treatment with the FITC-labeled pept-in (FIG. 8).

### Example 6: RAS-Targeting Pept-Ins Drive its Aggregation and Degradation in Cells

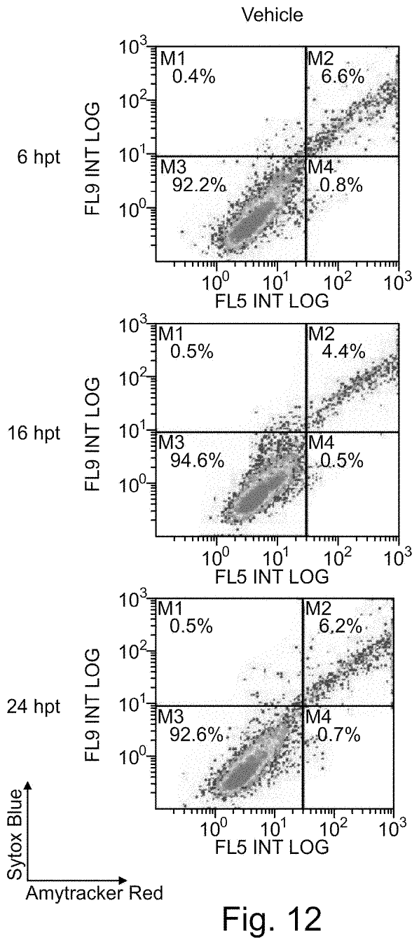

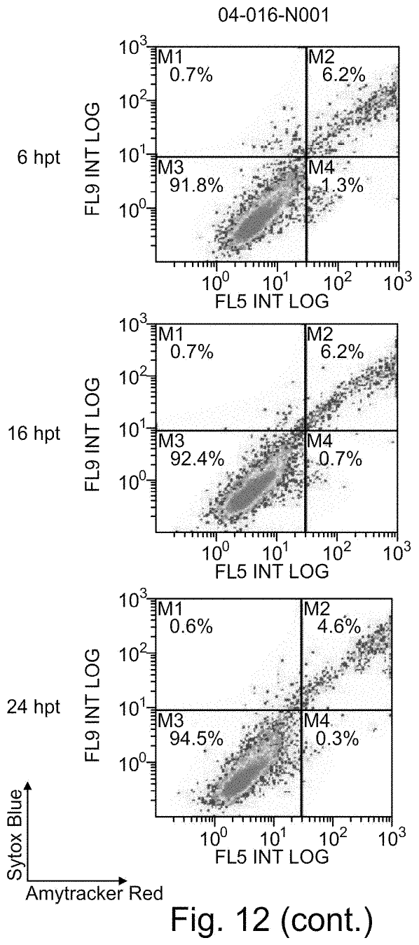

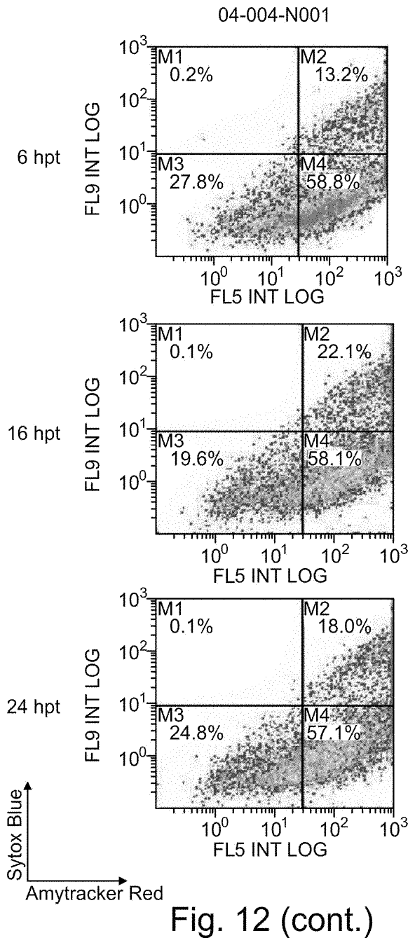

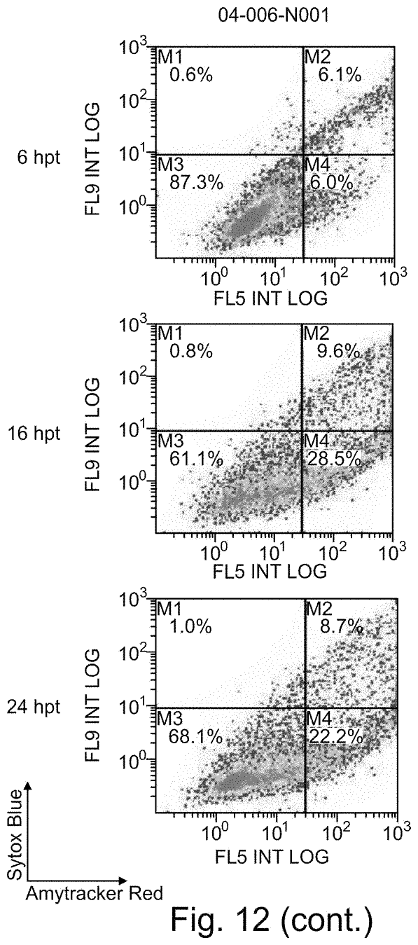

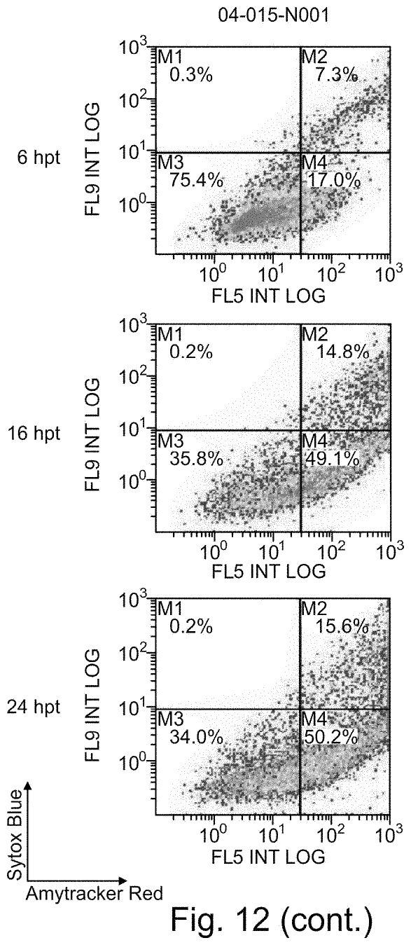

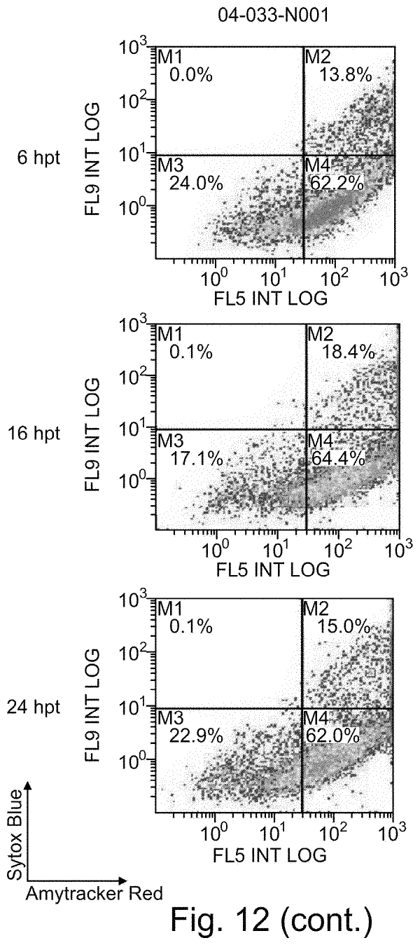

To assess whether treatment of tumor cells with the RAS-targeting pept-ins induces protein aggregation prior to inducing cell death, a flow cytometry assay was devised to monitor cell death in parallel with protein aggregation. To this end, NCI-H441 cells were treated for either 6, 16 or 24 hrs with a near-IC₅₀ dose of the RAS-targeting pept-ins (12.5 μM) or control conditions (vehicle and negative control pept-in). After treatment, cells were collected and stained for cell death using the Sytox™ Blue dye and for the presence of (amyloid-like) protein aggregates using the Amytracker™ Red dye. This analysis showed that for vehicle and control pept-in treated cells no significant cell death or protein aggregation was observed during the course of the experiment. However, upon treatment with the RAS-targeting pept-ins, protein aggregation was readily detected and appeared to progress over time. Furthermore, this increase in protein aggregation was paralleled with a slow increase in cell death, which appeared to be secondary to the occurrence of protein aggregation (FIG. 12).

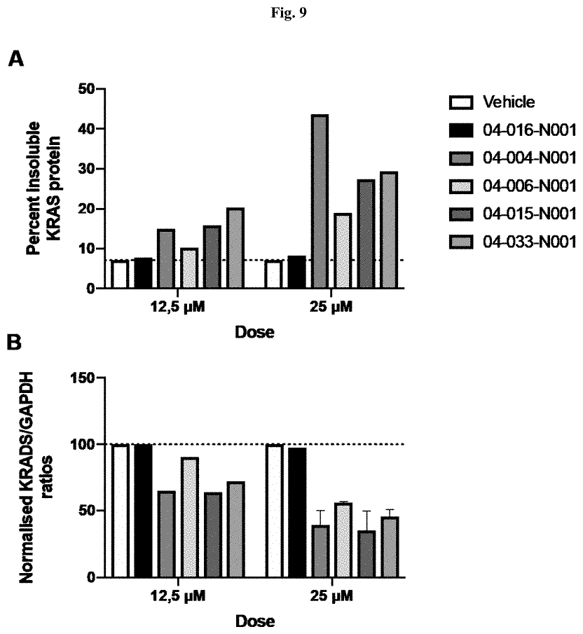

As the flow cytometry assay described above does not offer granularity as to whether the protein aggregation observed was affecting KRAS, we set out to assess KRAS aggregation in a solubility fractionation assay. To this end, NCI-H441 cells were treated with a near IC50 dose (12.5 μM) and a near 2×IC50 dose (25 μM) for 24 hrs. After treatment cells were lysed using a mild, non-denaturing buffer and proteins not soluble in this buffer were pelleted by centrifugation. Insoluble proteins were next solubilized using a strong chaotropic agent, i.e. 6M Urea. Using this approach, amyloid(-like) aggregates are expected to end up in the insoluble fraction. Both the soluble and insoluble fractions were resolved using SDS PAGE and probed for KRAS and GAPDH in a subsequent Western blot. This analysis showed that all biologically active RAS-targeting peptides dose-dependently increased the percentage of KRAS in the insoluble fraction while the percentage of insoluble KRAS was comparable between vehicle and negative control peptide treated samples, indicating that pept-in treatment indeed results in aggregation of the KRAS target protein. To complement these findings, we also quantified the total KRAS levels in these samples (i.e. sum of KRAS levels in the soluble and insoluble fraction for each treatment). Analysis of these data showed that total KRAS levels were also dose-dependently reduced in the samples treated with the biologically active RAS-targeting pept-ins (FIG. 9).

Together, these data show that also in cells the biologically active RAS-targeting pept-ins are able to interact with their intended target protein KRAS and induce its aggregation, as evidenced by the increase in insoluble KRAS protein upon treatment with the pept-ins. Furthermore, presumably, but without implying any limitation to a specific mechanism, as a secondary consequence to aggregation, total KRAS levels are also reduced after treatment with the active pept-ins.

### Example 7: RAS-Targeting Pept-Ins Reduce Tumor Growth in a Xenograft Model of KRAS G12V Mutant Cancer

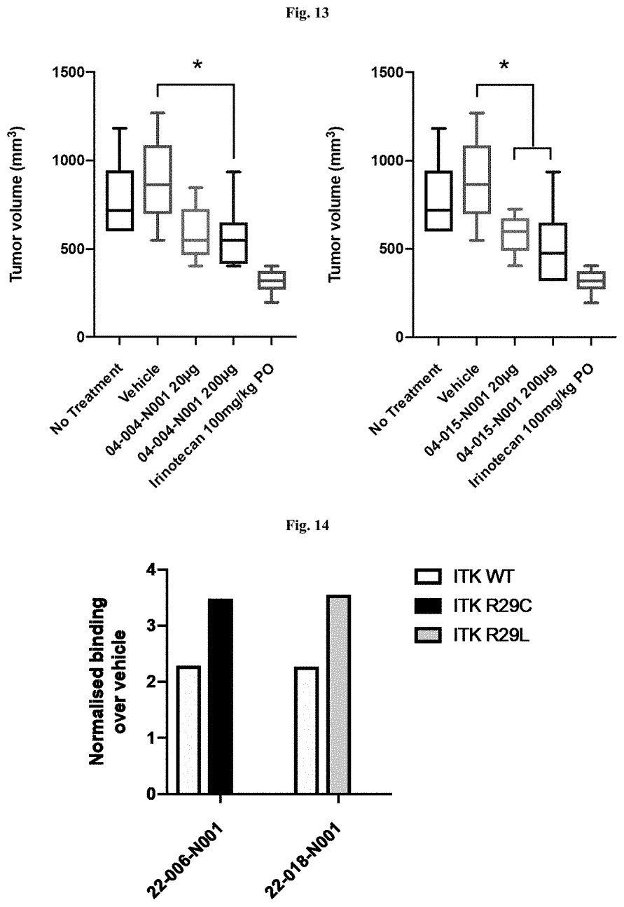

To assess whether the RAS-targeting pept-ins are able to attenuate growth of KRAS G12V-driven tumors in vivo, a subcutaneous xenograft model of human KRAS G12V colorectal cancer (SW620) was used. Once the tumors reached 100-150 mm³ in size, pept-ins were administered directly into the tumor mass by intratumoral injection three times per week during two weeks at two different doses (20 μg and 200 μg). From the set of pept-ins carrying a G12V-selective RAS APR window sequence (04-006-, 04-015-, and 04-033-N001), 04-015-N001 induced the strongest reduction in tumor growth, as evidenced by a significant reduction in average tumor volume for both the 20 μg and 200 μg dosing groups at day 22 after treatment started. Furthermore, a similar reduction in tumor growth was observed for 04-004-N001, carrying a wild-type RAS APR window sequence, which, however, was only significant for the 200 μg dosing group (FIG. 13).

### Example 8: Pept-Ins Targeting ITK R29L and R29C Mutants

Single amino acid substitution mutants (R29L and R29C) of ITK (Tyrosine-protein kinase ITK/TSK, Swissprot/UniProt acc. no. Q08881, sequence version 1) comprise an APR that is rendered longer by the mutations, and also displays an increased TANGO score, compared to the wild-type ITK protein (see table below, the sequences in rows 1-3 are denoted as SEQ ID NO: 29-31, respectively).

| | Start | N- | | c- | | |
|---|---|---|---|---|---|---|
| | position | ter | APR | ter | Score | Length |
| Mutation | APR | GKs | sequence | GKs | (%) | (aa) |
| WT | 29 | KVR | FFVLTKAS | DRH | 26.0231 | 13 |
| | | | LAYFE | | | |
| R29L | 27 | NFK | VLFFVLTK | DRH | 43.8347 | 15 |
| | | | ASLAYFE | | | |
| R29C | 27 | NFK | VCFFVLTK | DRH | 39.996 | 15 |
| | | | ASLAYFE | | | |

N-terminally biotinylated and C-terminally amidated pept-ins 22-006-N001, AKVCFFVKGSKVCFFVK (SEQ ID NO: 32), and 22-018-N001, AKVLFFVKGSKVLFFVK (SEQ ID NO: 33), were designed against the R29C and R29L ITK mutants, respectively. The mutated amino acid is shown in bold in the above sequences.

An in vitro translation approach was used to assess ITK mutant selective binding over wild-type for the 22-006-N001 and 22-018-N001 pept-ins. In particular, in vitro translation assays were performed using the PURExpress® In Vitro Protein Synthesis Kit (New England Biolabs) according to the manufacturer's instructions. Briefly, linear DNA fragments containing T7 promotor and terminator sequences flanking the DYKDDDDK (SEQ ID NO: 68)-tagged ITK coding sequence were generated using PCR and purified using the MinElute PCR Purification Kit (Qiagen). 250 ng of linear DNA was subsequently used for the in vitro translation reaction, which was performed for 2 hrs at 37° C. with shaking (1000 rpm). Indicated biotinylated pept-ins were mixed in the translation reactions from a 5 mM stock solution in 6M Urea to a final concentration of 10 μM. Upon completion of the translation reaction, biotinylated pept-ins were captured from the reaction mix using Streptavidin coated beads (Pierce) during 90 min at room temperature. Beads were next washed with TBS containing 0.1% Tween 20 and bound proteins were finally boiled off in 1×SDS loading dye (Bio-Rad) in TBS buffer. Proteins were resolved using Any kD 15-well Mini-PROTEAN gels (Bio-Rad) during SDS-PAGE and probed for ITK using a rabbit anti-DYKDDDDK (SEQ ID NO: 68) tag antibody (Cell Signaling 14793) after Western blotting.

The data in the bar graph in FIG. 14 shows fraction binding of total protein produced for each pept-in and target protein combination normalized over vehicle condition. Selective binding to the mutant over wild-type was observed for both 22-006-N001 and 22-018-N001 to ITK R29C and R29L, respectively.

1. A non-naturally occurring molecule capable of downregulating the amount or biological activity of a mutant or variant form of a protein, wherein:
a) the protein comprises a p-aggregation prone region (APR) and said APR is modified by the mutation or variation in the mutant or variant form of the protein; or
b) the mutation or variation introduces a de novo APR in the mutant or variant form of the protein not present in the protein;
and wherein the molecule is configured to specifically target the APR in the mutant or variant form of the protein.

2. The molecule according to claim 1, wherein the molecule is configured to form an intermolecular beta-sheet with the APR in the mutant or variant form of the protein but substantially not with the APR in the protein.

3. The molecule according to claim 1, wherein the intermolecular beta-sheet involves one or more of the amino acids which differ between the mutant or variant form of the protein and the protein.

4. The molecule according to claim 1, wherein the APR in the mutant or variant form of the protein differs from the APR in the protein in amino acid sequence or aggregation propensity, preferably in amino acid sequence, more preferably in amino acid sequence and aggregation propensity.

5. The molecule according to claim 4, wherein the aggregation propensity of the APR in the mutant or variant form of the protein is higher than the aggregation propensity of the APR in the protein.

6. The molecule according to claim 4, wherein:
a) the APR in the mutant or variant form of the protein has a higher proportion of hydrophobic amino acids than the APR in the protein;
b) the APR in the mutant or variant form of the protein has a lower proportion of amino acids that display low beta-sheet forming potential or a propensity to disrupt beta-sheets than the APR in the protein;
c) the APR in the mutant or variant form of the protein has a lower proportion of charged amino acids than the APR in the protein; and/or
d) the APR in the mutant or variant form of the protein is at least one amino acid longer than the APR in the protein, such as two, three or four amino acids longer.

7. The molecule according to claim 4, wherein:
a) the mutation or variation in the mutant or variant form of the protein modifies, such as substitutes, deletes or adds, one or more amino acids within the APR in the protein;
b) the mutation or variation in the mutant or variant form of the protein modifies, such as substitutes, deletes or adds, one or more amino acids within a region of between 1 and 10, preferably between 1 and 4 contiguous amino acids N-terminally adjacent to the APR in the protein, preferably whereby at least one amino acid of said region becomes part of the APR in the mutant or variant form of the protein; and/or
c) the mutation or variation in the mutant or variant form of the protein modifies, such as substitutes, deletes or adds, one or more amino acids within a region of between 1 and 10, preferably between 1 and 4 contiguous amino acids C-terminally adjacent to the APR in the protein, preferably whereby at least one amino acid of said region becomes part of the APR in the mutant or variant form of the protein.

8. The molecule according to claim 7, wherein the mutation or variation in said region N- or C-terminally adjacent to the APR in the protein:
a) increases the proportion of hydrophobic amino acids in said region;
b) reduces the proportion of amino acids that display low beta-sheet forming potential or a propensity to disrupt beta-sheets said region; and/or
c) reduces the proportion of charged amino acids in said region.

9. The molecule according to claim 1, wherein the molecule is able to decrease the solubility or to induce the aggregation or inclusion body formation of the mutant or variant form of the protein.

10. The molecule according to claim 2, wherein the molecule comprises an amino acid stretch, preferably a stretch of at least 6 contiguous amino acids, such as a stretch of 6 to 10 contiguous amino acids, which participates in the intermolecular beta-sheet with the APR in the mutant or variant form of the protein.

11. The molecule according to claim 10, wherein said stretch comprised by the molecule corresponds to an amino acid stretch, preferably to a stretch of at least 6 contiguous amino acids, such as a stretch of 6 to 10 contiguous amino acids, comprised by the APR in the mutant or variant form of the protein, preferably wherein:
a) the amino acid sequence of the stretch comprised by the molecule is identical to the stretch comprised by the APR;
b) the amino acid sequence of the stretch comprised by the molecule is at least 80% identical to the amino acid sequence of the stretch comprised by the APR;
c) the amino acid sequence of the stretch comprised by the molecule differs from the amino acid sequence of the stretch comprised by the APR by at most 3, preferably at most 2, and more preferably at most 1 amino acid substitutions;
d) the amino acid sequence of the stretch comprised by the molecule displays the degree of sequence identity to the amino acid sequence of the stretch comprised by the APR as set forth in any one of a) to c), and all amino acids of the molecule stretch are L-amino acids;
e) the amino acid sequence of the stretch comprised by the molecule displays the degree of sequence identity to the amino acid sequence of the stretch comprised by the APR as set forth in any one of a) to c), and at least one amino acid of the former stretch is a D-amino acid;
f) the amino acid sequence of the stretch comprised by the molecule displays the degree of sequence identity to the amino acid sequence of the stretch comprised by the APR as set forth in any one of a) to c), and at least one amino acid of the former stretch is replaced by an analogue of the respective amino acid; or
g) the amino acid sequence of the stretch comprised by the molecule displays the degree of sequence identity to the amino acid sequence of the stretch comprised by the APR as set forth in any one of a) to c), and at least one amino acid of the former stretch is a D-amino acid and at least one amino acid of the former stretch is replaced by an analogue of the respective amino acid.

12. The molecule according to claim 10, wherein the molecule comprises two or more, preferably two, said amino acid stretches, which are identical or different.

13. The molecule according to claim 10, wherein the amino acid stretch or stretches are each independently flanked, on each end independently, by one or more amino acids that display low beta-sheet forming potential or a propensity to disrupt beta-sheets.

14. The molecule according to claim 10, wherein the molecule comprises the structure:
a) NGK1-P1-CGK1,
b) NGK1-P1-CGK1-Z1-NGK2-P2-CGK2,
c) NGK1-P1-CGK1-Z1-NGK2-P2-CGK2-Z2-NGK3-P3-CGK3, or
d) NGK1-P1-CGK1-Z1-NGK2-P2-CGK2-Z2-NGK3-P3-CGK3-Z3-NGK4-P4-CGK4,
wherein:
P1 to P4 each independently denote the amino acid stretch that participates in the intermolecular beta-sheet,
NGK1 to NGK4 and CGK1 to CGK4 each independently denote 1 to 4 contiguous amino acids that display low beta-sheet forming potential or a propensity to disrupt beta-sheets, such as 1 to 4 contiguous amino acids selected from the group consisting of R, K, D, E, P, N, S, H, G, Q, and A, D-isomers and/or analogues thereof, and combinations thereof, preferably 1 to 4 contiguous amino acids selected from the group consisting of R, K, D, E, P, N, S, H, G, and Q, D-isomers and/or analogues thereof, and combinations thereof, more preferably 1 to 4 contiguous amino acids selected from the group consisting of R, K, D, E, and P, D-isomers and/or analogues thereof, and combinations thereof, and
Z1 to Z3 each independently denote a direct bond or preferably a linker.

15. The molecule according to claim 1, wherein the mutation or variation is a germline or somatic mutation or variation.

16. The molecule according to claim 1, wherein the mutant or variant form of the protein is causative of or associated with a disease.

17. The molecule according to claim 16, wherein the disease is a neoplastic disease, particularly cancer.

18. The molecule according to claim 17, wherein the protein is a proto-oncogene and the mutant or variant form of the protein is an oncogene.

19. A method of treating a neoplastic disease in a subject, wherein the neoplastic disease is caused by a mutant or a variant form of a protein that is causative of or associated with the disease, the method comprising administering a) the molecule according to claim 1, or b) a nucleic acid encoding said molecule, wherein the molecule is a polypeptide.

20. (canceled)

21. A pharmaceutical composition comprising
a) the molecule according to claim 1, or b) a nucleic acid encoding said molecule,
wherein the molecule is a polypeptide.

22. An in vitro method for downregulating the amount or biological activity of a mutant or variant form of a protein in a cell expressing, preferably endogenously expressing, the mutant or variant form of the protein, the method comprising contacting the cell with a non-naturally occurring molecule capable of downregulating the amount or biological activity of the mutant or variant form of the protein, wherein:
a) the protein comprises a p-aggregation prone region (APR) and said APR is modified by the mutation or variation in the mutant or variant form of the protein; or
b) the mutation or variation introduces a de novo APR in the mutant or variant form of the protein not present in the protein;
and wherein the molecule is configured to specifically target the APR in the mutant or variant form of the protein; or
comprising contacting the cell with a nucleic acid encoding the molecule, wherein the molecule is a polypeptide.

23. A method for downregulating the amount or biological activity of a mutant or variant form of a protein in an organism expressing, preferably endogenously expressing, the mutant or variant form of the protein, the method comprising administering to the organism a non-naturally occurring molecule capable of downregulating the amount or biological activity of the mutant or variant form of the protein, wherein:
a) the protein comprises a p-aggregation prone region (APR) and said APR is modified by the mutation or variation in the mutant or variant form of the protein; or
b) the mutation or variation introduces a de novo APR in the mutant or variant form of the protein not present in the protein;
and wherein the molecule is configured to specifically target the APR in the mutant or variant form of the protein; or
comprising contacting the cell with a nucleic acid encoding the molecule, wherein the molecule is a polypeptide.

24. (canceled)

25. The method according to claim 22, wherein the cell is a bacterial cell, a fungal cell, including a yeast cell or a mould cell, a protist cell, a plant cell, or an animal cell, including a non-human mammal cell or a human cell.

26. The method according to claim 23, wherein the organism is a bacterium, a fungus, including yeast or mould, a plant, or an animal.


SEQUENCE LISTING (103 sequences)

SEQ ID NO: 1 - protein, 189 aa, Homo sapiens [sequence omitted]

SEQ ID NO: 2 - protein, 11 aa, Homo sapiens
TEYKLVVVGAG

SEQ ID NO: 3 - protein, 14 aa, Homo sapiens
TEYKLVVVGAVGVG

SEQ ID NO: 4 - protein, 13 aa, Homo sapiens
TEYKLVVVGACGV

SEQ ID NO: 5 - protein, 13 aa, Homo sapiens
TEYKLVVVGAAGV

SEQ ID NO: 6 - protein, 12 aa, Homo sapiens
TEYKLVVVGASG

SEQ ID NO: 7 - protein, 13 aa, Homo sapiens
TEYKLVVVGAGCV

SEQ ID NO: 8 - protein, 14 aa, Homo sapiens
TEYKLVVVGAGVVG

SEQ ID NO: 9 - protein, 12 aa, Homo sapiens
TEYKLVVVGAGS

SEQ ID NO: 10 - protein, 10 aa, Homo sapiens
LVVVGAVGVG

SEQ ID NO: 11 - protein, 9 aa, Homo sapiens
LVVVGACGV

SEQ ID NO: 12 - protein, 9 aa, Homo sapiens
LVVVGAAGV

SEQ ID NO: 13 - protein, 8 aa, Homo sapiens
LVVVGASG

SEQ ID NO: 14 - protein, 9 aa, Artificial seqquence
  Linker
LVVVGAGCV

SEQ ID NO: 15 - protein, 10 aa, Homo sapiens
LVVVGAGVVG

SEQ ID NO: 16 - protein, 8 aa, Homo sapiens
LVVVGAGS

SEQ ID NO: 17 - protein, 18 aa, Artificial sequence
  RAS pept-in
KVVVGAVKGSKVVVGAVK

SEQ ID NO: 18 - protein, 20 aa, Artificial sequence
  RAS pept-in
KLVVVGAVKGSKLVVVGAVK

SEQ ID NO: 19 - protein, 20 aa, Artificial sequence
  RAS pept-in
KVVVGAVGKGSKVVVGAVGK

SEQ ID NO: 20 - protein, 24 aa, Artificial sequence
  RAS pept-in
KVVVGAVGVGKGSKVVVGAVGVGK

SEQ ID NO: 21 - protein, 7 aa, Homo sapiens
VLTSGIF

SEQ ID NO: 22 - protein, 8 aa, Homo sapiens
CVLTSGIF

SEQ ID NO: 23 - protein, 9 aa, Homo sapiens
CLVLTSGIF

SEQ ID NO: 24 - protein, 11 aa, Homo sapiens
YIVGFYGAFYS

SEQ ID NO: 25 - protein, 13 aa, Homo sapiens
SLYIVGFYGAFYS

SEQ ID NO: 26 - protein, 7 aa, Homo sapiens
ILGGTVF

SEQ ID NO: 27 - protein, 11 aa, Homo sapiens
TILNILGGTVF

SEQ ID NO: 28 - protein, 11 aa, Homo sapiens
TIWNILGGTVF

SEQ ID NO: 29 - protein, 13 aa, Homo sapiens
FFVLTKASLAYFE

SEQ ID NO: 30 - protein, 15 aa, Homo sapiens
VLFFVLTKASLAYFE

SEQ ID NO: 31 - protein, 15 aa, Homo sapiens
VCFFVLTKASLAYFE

SEQ ID NO: 32 - protein, 17 aa, Artificial sequence
  ITK pept-in
AKVCFFVKGSKVCFFVK

SEQ ID NO: 33 - protein, 17 aa, Artificial sequence
  ITK pept-in
AKVLFFVKGSKVLFFVK

SEQ ID NO: 34 - protein, 5 aa, Homo sapiens
FATVV

SEQ ID NO: 35 - protein, 5 aa, Homo sapiens
FVTVV

SEQ ID NO: 36 - protein, 5 aa, Homo sapiens
FGTVV

SEQ ID NO: 37 - protein, 8 aa, Homo sapiens
YTFGVSCV

SEQ ID NO: 38 - protein, 6 aa, Homo sapiens
SFVTVY

SEQ ID NO: 39 - protein, 6 aa, Homo sapiens
SFLTVY

SEQ ID NO: 40 - protein, 7 aa, Homo sapiens
ALTIQLI

SEQ ID NO: 41 - protein, 9 aa, Homo sapiens
GFLCVFAIN

SEQ ID NO: 42 - protein, 5 aa, Homo sapiens
MVLVG

SEQ ID NO: 43 - protein, 6 aa, Homo sapiens
AFYTLV

SEQ ID NO: 44 - protein, 10 aa, Homo sapiens
TEYKLVVVGA

SEQ ID NO: 45 - protein, 4 aa, Artificial sequence
  Gatekeeper region
KKKK

SEQ ID NO: 46 - protein, 4 aa, Artificial sequence
  Gatekeeper region
RRRR

SEQ ID NO: 47 - protein, 4 aa, Artificial sequence
  Gatekeeper region
DDDD

SEQ ID NO: 48 - protein, 4 aa, Artificial sequence
  Gatekeeper region
EEEE

SEQ ID NO: 49 - protein, 4 aa, Artificial sequence
  Gatekeeper region
KRKR

SEQ ID NO: 50 - protein, 4 aa, Artificial sequence
  Gatekeeper region
KRRK

SEQ ID NO: 51 - protein, 4 aa, Artificial sequence
  Gatekeeper region
RKKR

SEQ ID NO: 52 - protein, 4 aa, Artificial sequence
  Gatekeeper region
DEDE

SEQ ID NO: 53 - protein, 4 aa, Artificial sequence
  Gatekeeper region
DEED

SEQ ID NO: 54 - protein, 4 aa, Artificial sequence
  Gatekeeper region
EDDE

SEQ ID NO: 55 - protein, 4 aa, Artificial sequence
  Linker
GGGG

SEQ ID NO: 56 - protein, 4 aa, Artificial sequence
  Linker
SSSS

SEQ ID NO: 57 - protein, 4 aa, Artificial sequence
  Linker
GGGS

SEQ ID NO: 58 - protein, 4 aa, Artificial sequence
  Linker
GGSG

SEQ ID NO: 59 - protein, 4 aa, Artificial sequence
  Linker
GSGG

SEQ ID NO: 60 - protein, 4 aa, Artificial sequence
  Linker
SGGG

SEQ ID NO: 61 - protein, 4 aa, Artificial sequence
  Linker
GGSS

SEQ ID NO: 62 - protein, 4 aa, Artificial sequence
  Linker
GSSG

SEQ ID NO: 63 - protein, 4 aa, Artificial sequence
  Linker
SSGG

SEQ ID NO: 64 - protein, 4 aa, Artificial sequence
  Linker
SGSG

SEQ ID NO: 65 - protein, 5 aa, Artificial sequence
  Linker
GSGSG

SEQ ID NO: 66 - protein, 5 aa, Artificial sequence
  Linker
SGSGS

SEQ ID NO: 67 - protein, 4 aa, Artificial sequence
  Linker
PPPP

SEQ ID NO: 68 - protein, 8 aa, Artificial sequence
  FLAG tag
DYKDDDDK

SEQ ID NO: 69 - protein, 18 aa, Artificial sequence
  RAS targeting molecule
KLSVFAIKGSKLSVFAIK

SEQ ID NO: 70 - protein, 4 aa, Homo sapiens
GSGS

SEQ ID NO: 71 - protein, 6 aa, Homo sapiens
LSVFAI

SEQ ID NO: 72 - protein, 6 aa, Homo sapiens
VVVGAV

SEQ ID NO: 73 - protein, 7 aa, Homo sapiens
VVVGAVG

SEQ ID NO: 74 - protein, 7 aa, Homo sapiens
VVVGAVG

SEQ ID NO: 75 - protein, 18 aa, Artificial sequence
  RAS pept-in
  MISC_FEATURE (1)..(1): D-lysine
  MISC_FEATURE (18)..(18): D-lysine
XLSVFAIKGSKLSVFAIX

SEQ ID NO: 76 - protein, 18 aa, Artificial sequence
  RAS pept-in
  MISC_FEATURE (1)..(1): D-lysine
  MISC_FEATURE (18)..(18): D-lysine
XVVVGAVKGSKVVVGAVX

SEQ ID NO: 77 - protein, 20 aa, Artificial sequence
  RAS pept-in
  MISC_FEATURE (1)..(1): D-lysine
  MISC_FEATURE (20)..(20): D-lysine
XVVVGAVGKGSKVVVGAVGX

SEQ ID NO: 78 - protein, 24 aa, Artificial sequence
  RAS pept-in
  MISC_FEATURE (1)..(1): D-lysine
  MISC_FEATURE (24)..(24): D-lysine
XVVVGAVGVGKGSKVVVGAVGVGX

SEQ ID NO: 79 - protein, 18 aa, Artificial sequence
  RAS pept-in
  MOD_RES (1)..(1): Dpm
  MOD_RES (18)..(18): Dpm
XLSVFAIKGSKLSVFAIX

SEQ ID NO: 80 - protein, 18 aa, Artificial sequence
  RAS pept-in
  MOD_RES (1)..(1): Dpm
  MOD_RES (18)..(18): Dpm
XVVVGAVKGSKVVVGAVX

SEQ ID NO: 81 - protein, 20 aa, Artificial sequence
  RAS pept-in
  MOD_RES (1)..(1): Dpm
  MOD_RES (20)..(20): Dpm
XVVVGAVGKGSKVVVGAVGX

SEQ ID NO: 82 - protein, 24 aa, Artificial sequence
  RAS pept-in
  MOD_RES (1)..(1): Dpm
  MOD_RES (24)..(24): Dpm
XVVVGAVGVGKGSKVVVGAVGVGX

SEQ ID NO: 83 - protein, 18 aa, Artificial sequence
  RAS pept-in
  MISC_FEATURE (1)..(1): citrulline
XVVVGAVKGSKVVVGAVK

SEQ ID NO: 84 - protein, 17 aa, Artificial sequence
  RAS pept-in
  MISC_FEATURE (8)..(8): citrulline
KVVVGAVGSKVVVGAVK

SEQ ID NO: 85 - protein, 18 aa, Artificial sequence
  RAS pept-in
AVVVGAVKGSKVVVGAVK

SEQ ID NO: 86 - protein, 18 aa, Artificial sequence
  RAS pept-in
KVVVGAVAGSKVVVGAVK

SEQ ID NO: 87 - protein, 18 aa, Artificial sequence
  RAS pept-in
KVVVGAVKGSAVVVGAVK

SEQ ID NO: 88 - protein, 18 aa, Artificial sequence
  RAS pept-in
KVVVGAVKGSKVVVGAVA

SEQ ID NO: 89 - protein, 18 aa, Artificial sequence
  RAS pept-in
AVVVGAVKGSAVVVGAVK

SEQ ID NO: 90 - protein, 18 aa, Artificial sequence
  RAS pept-in
KVVVGAVAGSKVVVGAVA

SEQ ID NO: 91 - protein, 18 aa, Artificial sequence
  RAS pept-in
AVVVGAVAGSKVVVGAVK

SEQ ID NO: 92 - protein, 18 aa, Artificial sequence
  RAS pept-in
KVVVGAVKASKVVVGAVK

SEQ ID NO: 93 - protein, 18 aa, Artificial sequence
  RAS pept-in
KVVVGAVKGAKVVVGAVK

SEQ ID NO: 94 - protein, 20 aa, Artificial sequence
  RAS pept-in
KVVVGAVGKGFKVVVGAVGK

SEQ ID NO: 95 - protein, 20 aa, Artificial sequence
  RAS pept-in
KVVVGAVGKFFKVVVGAVGK

SEQ ID NO: 96 - protein, 16 aa, Artificial sequence
  RAS pept-in
KLSVFAIKKLSVFAIK

SEQ ID NO: 97 - protein, 22 aa, Artificial sequence
  RAS pept-in
KVVVGAVGVGKKVVVGAVGVGK

SEQ ID NO: 98 - protein, 18 aa, Artificial sequence
  RAS pept-in
  MISC_FEATURE (1)..(1): D-lysine
  MISC_FEATURE (2)..(2): D-leucine
  MISC_FEATURE (3)..(3): D-serine
  MISC_FEATURE (4)..(4): D-valine
  MISC_FEATURE (5)..(5): D-phenylalanine
  MISC_FEATURE (6)..(6): D-alanine
  MISC_FEATURE (7)..(7): D-isoleucine
  MISC_FEATURE (8)..(8): D-lysine
  MISC_FEATURE (10)..(10): D-serine
  MISC_FEATURE (11)..(11): D-lysine
  MISC_FEATURE (12)..(12): D-leucine
  MISC_FEATURE (13)..(13): D-serine
  MISC_FEATURE (14)..(14): D-valine
  MISC_FEATURE (15)..(15): D-phenylalanine
  MISC_FEATURE (16)..(16): D-alanine
  MISC_FEATURE (17)..(17): D-isoleucine
  MISC_FEATURE (18)..(18): D-lysine
XXXXXXXXGXXXXXXXXX

SEQ ID NO: 99 - protein, 18 aa, Artificial sequence
  RAS pept-in
  MISC_FEATURE (1)..(1): D-lysine
  MISC_FEATURE (2)..(2): D-valine
  MISC_FEATURE (3)..(3): D-valine
  MISC_FEATURE (4)..(4): D-valine
  MISC_FEATURE (6)..(6): D-alanine
  MISC_FEATURE (7)..(7): D-valine
  MISC_FEATURE (8)..(8): D-lysine
  MISC_FEATURE (10)..(10): D-serine
  MISC_FEATURE (11)..(11): D-lysine
  MISC_FEATURE (12)..(12): D-valine
  MISC_FEATURE (13)..(13): D-valine
  MISC_FEATURE (14)..(14): D-valine
  MISC_FEATURE (16)..(16): D-alanine
  MISC_FEATURE (17)..(17): D-valine
  MISC_FEATURE (18)..(18): D-lysine
XXXXGXXXGXXXXXGXXX

SEQ ID NO: 100 - protein, 24 aa, Artificial sequence
  RAS pept-in
  MISC_FEATURE (1)..(1): D-lysine
  MISC_FEATURE (2)..(2): D-valine
  MISC_FEATURE (3)..(3): D-valine
  MISC_FEATURE (4)..(4): D-valine
  MISC_FEATURE (6)..(6): D-alanine
  MISC_FEATURE (7)..(7): D-valine
  MISC_FEATURE (9)..(9): D-valine
  MISC_FEATURE (11)..(11): D-lysine
  MISC_FEATURE (13)..(13): D-serine
  MISC_FEATURE (14)..(14): D-lysine
  MISC_FEATURE (15)..(15): D-valine
  MISC_FEATURE (16)..(16): D-valine
  MISC_FEATURE (17)..(17): D-valine
  MISC_FEATURE (19)..(19): D-alanine
  MISC_FEATURE (20)..(20): D-valine
  MISC_FEATURE (22)..(22): D-valine
  MISC_FEATURE (24)..(24): D-lysine
XXXXGXXGXGXGXXXXXGXXGXGX

SEQ ID NO: 101 - protein, 10 aa, Artificial sequence
  peptide
KLVVVGAVGV

SEQ ID NO: 102 - protein, 17 aa, Artificial sequence
  peptide
KLVVVGAVGVGKSALTI

SEQ ID NO: 103 - protein, 13 aa, Artificial sequence
  peptide
KLVVVGAVGVGKS